(19) United States
(12) Patent Application Publication
(10) Pub. No.: US 2025/0034561 A1
(43) Pub. Date: Jan. 30, 2025

(54) Novel Compositions for Conjugating Oligonucleotides and Carbohydrates

(71) Applicant: ONEGLOBE HOLDINGS LIMITED, Hong Kong (CN)

(72) Inventors: Chiang J. LI, Cambridge, MA (US); Chen CAO; Li GUI; Praveen POGULA, Hopkinton, MA (US); Xiangao SUN, Brookline, MA (US); Danielle RAND; Qi LIU

(21) Appl. No.: 18/277,846

(22) PCT Filed: Feb. 18, 2022
(86) PCT No.: PCT/IB2022/000074

§ 371 Date: Aug. 18, 2023

Related U.S. Application Data
(60) Provisional application No. 63/151,060, filed on Feb. 18, 2021.

(51) Int. Cl. C12N 15/113; C07H 21/02

(52) U.S. Cl. CPC C12N 15/113; C07H 21/02; C12N 2310/11; C12N 2310/14; C12N 2310/141; C12N 2310/351

(57) ABSTRACT

The invention provides novel compositions and linking configurations for an oligonucleotide to be conjugated to a ligand for targeted in vivo delivery of an oligonucleotide. The invention further provides use of the resulting compounds and pharmaceutical compositions thereof in preparation of a medicament effective for treating a disease or condition.

### CROSS-REFERENCE TO RELATED APPLICATIONS

This application claims priority to and the benefit of co-pending U.S. provisional patent application Ser. No. 63/151,060, filed Feb. 18, 2021, which application is incorporated herein by reference in its entirety.

### FIELD OF THE INVENTION

The invention relates to novel compositions and processes that can be used in conjugating carbohydrate ligands with oligonucleotides intended for biomedical applications.

### BACKGROUND OF THE INVENTION

Gene-modulating and, in particular, gene-silencing oligonucleotides have been the focus of many research and development efforts as these strings of nucleotides hold great promise for treating or preventing many diseases and for modulating physiological conditions. Examples of such oligonucleotides include short/small interfering RNA (siRNA), asymmetric short/small interfering RNA (aiRNA), antisense oligonucleotide (ASO), and micro-RNA (miRNA).

RNA interference (RNAi) works through short, double-stranded RNA (dsRNA) duplexes called siRNA in a gene-specific fashion in many organisms. The siRNAs have a well-defined structure of symmetric, short (usually 20-24 base pairs) dsRNA duplex having phosphorylated 5′ ends and hydroxylated 3′ ends that form two 3′ overhangs of equal lengths. Gene modulation is mediated through a multi-protein RNA-induced silencing complex (RISC), which binds, unwinds, and incorporates the anti-sense siRNA strand from the siRNA duplex, and then recognizes and targets complementary messenger RNAs (mRNAs) for cleavage thereby reducing its gene expression in a post-transcriptional fashion.

Relatively new to the scene, aiRNA was developed to overcome off-target effects mediated by sense strand of the symmetrically configured canonical siRNA as well as other off-target mechanisms of siRNA (See PCT Patent Publication WO2009029688). AiRNAs are designed to include short RNA duplex where the lengths of the two RNA strands are not equal, hence “asymmetric.” For example, an aiRNA can include a first strand that is 18-23 nucleotides long and a second that is 12-17 nucleotides long, forming a duplex where the first strand might have a 3′ overhang of 1-9 nucleotides and a 5′ overhang of 0-8 nucleotides. The aiRNA technology can be used in all areas where current siRNA or short-hairpin RNA (shRNA) are being applied including biology research, R&D research in biotechnology and pharmaceutical industry, and RNAi-based therapies.

Antisense technology is a highly selective gene silencing technology based upon a concept originally proposed in 1978 (Zamecnik P. C. et al., 1978). Generally, the principle behind the ASO technology is that an antisense oligonucleotide hybridizes to a target nucleic acid and modulates gene expression through post-transcriptional mechanisms. The mechanisms can be broadly categorized as: (1) occupancy only without promoting RNA degradation, in which the binding of the ASO leads to translational arrest, inhibition of splicing, or induction of alternatively spliced variants, or (2) occupancy-induced destabilization, in which the binding of the ASO promotes degradation of the RNA through endogenous enzymes, such as ribonuclease H1 (RNase H1); and (3) increased translation: ASO can block upstream open reading frames (uORFs) or other inhibitory elements in the 5′UTR, increasing translation efficiency (Stanley T. Crooke et al., 2008; C. Frank Bennett, 2010; Richard G. Lee, 2013; Stanley T. Crooke, 2017). The typical structure of an ASO is a single-stranded deoxyribonucleotide sequence with sulfur chemistry modification, known as phosphorothioate. After 40 years of research, antisense technology has been improved through various chemical modifications of the single stranded oligonucleotide.

A miRNA molecule normally derives from noncoding regions of RNA transcripts that fold back onto themselves to form hairpins. After having been processed from its precursors through various cellular machineries, a mature miRNA is a small (about 22 nucleotides) RNA molecule found in plants, animal and some viruses that regulate gene expression through post-transcriptional silencing.

Therapeutics based on these and other nucleic acids provide promising solutions to a variety of diseases, including non-druggable targets. However, despite the advances in application of oligonucleotides and oligonucleotide analogs as therapeutics, there continues to exist great needs for enhancing key pharmacological properties of these therapeutic oligonucleotides in areas such as serum stability, delivery to the intended organ or cell population, and uptake across cellular membranes.

Preferred delivery of therapeutic oligonucleotides to cells in vivo, e.g., in a mammalian body such as a human's, requires specific targeting and protection from the extracellular environment inside the body including from proteins in the serum. A method that researchers have employed to achieve specific targeting is to conjugate a targeting moiety to the oligonucleotides to direct therapeutic oligonucleotides to the desired target site.

One way to improve specificity in delivery is by taking advantage of receptor mediated endocytic activities that already exist in the body. The mechanism of uptake involves the movement of molecules bound to cell membrane receptors across the membrane and into the cell via invagination of the membrane structure or by fusion of the delivery system with the cell membrane. This process is initiated via activation of a cell-surface or membrane receptor following binding of a specific ligand to the receptor. Therefore, by conjugating a drug candidate to a targeting moiety that targets such cell surface receptor(s), one can effectively borrow the innate endocytic pathways for drug delivery. Many receptor-mediated endocytic systems are known and have been studied, including those that recognize sugars including galactose, mannose, mannose-6-phosphate, peptides and proteins such as transferrin, asialoglycoprotein, vitamin B12, insulin and epidermal growth factor (EGF). In particular, the Asialoglycoprotein receptor (ASGP-R) is a highly abundant receptor on hepatocytes. The ASGP-R shows a 50-fold higher affinity for N-Acetyl-D-Galactosylamine (GalNAc) than for D-Gal. However, it has been reported that in using this conjugation system, the linker structure design and various chemical attributes of the linker moieties are crucial in determining overall delivery efficiency, efficacy and safety of the conjugated oligonucleotides as well as in affecting the stability and manufacture challenges of the various therapeutic oligonucleotides.

Accordingly, a strong need remains for new and effective receptor-specific, ligand-conjugated nucleic acid complex designs for various biomedical applications.

### SUMMARY OF THE INVENTION

In first aspect, the invention relates to a compound, as a therapeutic agent, where an oligonucleotide is conjugated with at least one ligand, e.g., a carbohydrate ligand such as a monosaccharide, disaccharide, trisaccharide, tetrasaccharide, oligosaccharide, polysaccharide or their derivatives, which can target the compound to receptor cells in the liver that can facilitate endocytic uptake as discussed above.

These ligand-conjugated compounds target one or more organs or cell types, e.g., the parenchymal cells of the liver in a human. In one embodiment, the compound includes more than one carbohydrate ligand, preferably two or three. In another embodiment, the compound of the invention includes at least one (e.g., one, two or three or more) N-Acetyl-Galactosamine (GalNAc), N-Ac-Glucosamine (GluNAc), galactose, lactose, or mannose (e.g., mannose-6-phosphate). In yet another embodiment, the compound of the invention includes at least one (e.g., one, two or three or more) ligand selected from the group consisting of GalNAc, cholesterol, tocopherol, biotin, cyanine dyes, folic acid, RGDp, transferrin, anisamide, lactobionic acid, cRGD, hyaluronic acid, low molecular weight protamine, lipid derivatives, peptides, cyclic peptides, and heterocycles.

In second aspect, the invention provides ligand-conjugated compounds having novel structure:

Item 1, a compound having the structural formula (G-H1):

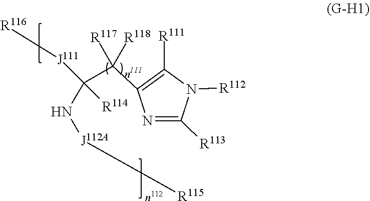

wherein:
R¹¹¹, R¹¹², R¹¹³ are each independently for each occurrence H, or R^{119A}; and at least one of R¹¹¹, R¹¹², R¹¹³ is R^{119A}.
R^{119A} comprising at least one ligand capable of docking to a cell surface receptor;
R¹¹⁴, R¹¹⁷, R¹¹⁸ are selected one or more from the group consisting of H, or, alkyl, aryl, heteroaryl, haloalkyl, —O alkyl, —O alkylphenyl, -alkyl-OH, —O haloalkyl, —S alkyl, —S alkylphenyl, -alkyl-SH, —S haloalkyl, halo, —OH, —SH, —NH₂, -alkyl-NH₂, —N(alkyl) (alkyl), —NH (alkyl), —N (alkyl) (alkylphenyl), —NH (alkylphenyl), cyano, nitro, —CO₂H, —C(O)O alkyl, —CON (alkyl) (alkyl), —CONH (alkyl), —CONH₂, —NHC(O) (alkyl), —NHC(O) (phenyl), —N(alkyl) C(O) (alkyl), —N(alkyl) C(O) (phenyl), —C(O) alkyl, —C(O) alkylphenyl, —C(O) haloalkyl, —OC(O) alkyl, —SO₂ (alkyl), —SO₂ (phenyl), —SO₂ (haloalkyl), —SO₂NH₂, —SO₂NH (alkyl), —SO₂NH (phenyl), —NHSO₂ (alkyl), —NHSO₂ (phenyl), and —NHSO₂ (haloalkyl);
R¹¹⁵, R¹¹⁶ are each independently for each occurrence OH, a protecting group for OH, a phosphate group, a phosphodiester group, an activated phosphate group, an activated phosphite group, a phosphoramidite, a solid support, —OP(Z′)(Z″)O-nucleoside, —OP(Z′)(Z″)O-oligonucleotide, a lipid, a PEG, a steroid, a polymer, —O-nucleotide, a nucleoside, —OP(Z′)(Z″)O—R^{119B}—OP(Z′″)(Z″″)O-oligonucleotide, or an oligonucleotide;
J¹¹¹, J¹¹², R^{119B} are each independently for each occurrence a spacer;
Z′, Z″, Z′″ and Z″″ are each independently for each occurrence O or S;
n¹¹¹, n¹¹² are each independently for each occurrence 1, 2, 3, 4, 5 or 6;
the oligonucleotide comprising natural or chemically modified nucleotides/nucleosides.

Item 2, the compound of item 1, wherein the compound having the structural formula (G-H1-01):

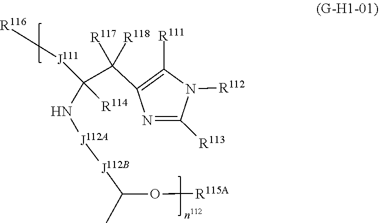

wherein:
J^{112A} is selected from the group consisting of: C(O), NH, O, S, OP(O)O, OP(S)O, CH═N and S(O)₂;
J^{112B} is selected from a alkylene of 1 to 10 carbon atoms;
R^{115A} is a solid support or H;
R¹¹⁶ is selected from the group consisting of: OH, a protecting group for OH, a phosphate group, a phosphodiester group, an activated phosphate group, an activated phosphite group, a phosphoramidite, —OP(Z′)(Z″)O-nucleoside, —OP(Z′)(Z″)O-oligonucleotide, a lipid, a PEG, a steroid, a polymer, —O-nucleotide, a nucleoside, —OP(Z′)(Z″)O—R^{119B}—OP(Z′″)(Z″″)O-oligonucleotide, or an oligonucleotide.
   Optionally, J^{112B} is selected from a straight alkylene of 1 to 10 carbon atoms.

Item 3, the compound of item 1 or item 2, wherein n¹¹² is 1.

Item 4, the compound of any one of items 1-3, wherein R¹¹⁶ comprises an oligonucleotide.

Item 5, the compound of item 4, wherein the compound having the structural formula (G-H1-02):

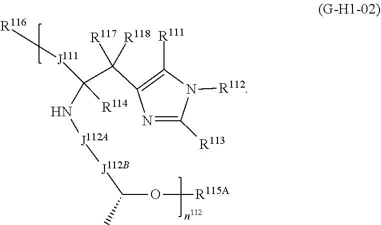

Item 6, the compound of item 4, wherein the compound having the structural formula (G-H1-03):

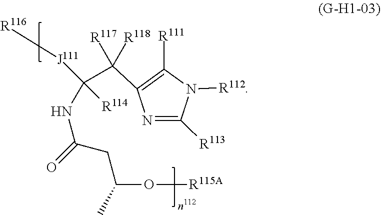

Item 7, the compound of item 4, wherein the compound having the structural formula (G-H1-04):

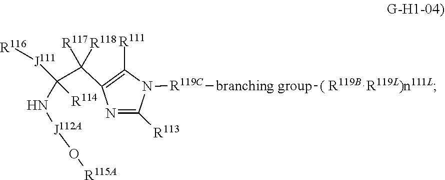

Wherein:
R^{119C} is selected from —C(O)—C₅-C₈ straight alkylene-NHCO—CH₂— or —C(O)—C₈-C₁₁ straight alkylene-;
R^{119L} is independently selected from a ligand capable of docking to a cell surface receptor;
n^{111L} is selected from 1, 2, 3, 4 or 5.

Item 8, the compound of item 7, wherein:
J¹¹¹, J¹¹², R^{119B} are independently selected from a alkylene of 1 to 30 carbon atoms, wherein one or more carbon atoms are optionally replaced with any one or more substituent of the group consisting of: C(O), NH, O, S, OP(O)O, OP(S)O, CH═N, S(O)₂, C₂-C₁₀ alkenylene, C₂-C₁₀ alkynylene, C₆-C₁₀ arylene, C₃-C₁₈ heterocyclylene, and C₅-C₁₀ heteroarylene, and wherein R^{119B} is optionally not substituted or substituted by R^{119D};
R¹¹⁴, R¹¹⁷, R¹¹⁸, R^{119D} are selected one or more from the group consisting of H, or, C₁-C₁₀ alkyl, C₆-C₁₀ aryl, C₅-C₁₀ heteroaryl, C₁-C₁₀ haloalkyl, —OC₁-C₁₀ alkyl, —OC₁-C₁₀ alkylphenyl, —C₁-C₁₀ alkyl-OH, —OC₁-C₁₀ haloalkyl, —SC₁-C₁₀ alkyl, —SC₁-C₁₀ alkylphenyl, —C₁-C₁₀ alkyl-SH, —SC₁-C₁₀ haloalkyl, halo, —OH, —SH, —NH₂, —C₁-C₁₀ alkyl-NH₂, —N(C₁-C₁₀ alkyl) (C₁-C₁₀ alkyl), —NH(C₁-C₁₀ alkyl), —N(C₁-C₁₀ alkyl)(C₁-C₁₀ alkylphenyl), —NH(C₁-C₁₀ alkylphenyl), cyano, nitro, —CO₂H, —C(O)OC₁-C₁₀ alkyl, —CON(C₁-C₁₀ alkyl)(C₁-C₁₀ alkyl), —CONH(C₁-C₁₀ alkyl), —CONH₂, —NHC(O) (C₁-C₁₀ alkyl), —NHC(O) (phenyl), —N(C₁-C₁₀ alkyl) C(O) (C₁-C₁₀ alkyl), —N(C₁-C₁₀ alkyl) C(O) (phenyl), —C(O) C₁-C₁₀ alkyl, —C(O) C₁-C₁₀ alkylphenyl, —C(O) C₁-C₁₀ haloalkyl, —OC(O)C₁-C₁₀ alkyl, —SO₂ (C₁-C₁₀ alkyl), —SO₂ (phenyl), —SO₂ (C₁-C₁₀ haloalkyl), —SO₂NH₂, —SO₂NH(C₁-C₁₀ alkyl), —SO₂NH (phenyl), —NHSO₂(C₁-C₁₀ alkyl), —NHSO₂ (phenyl), and —NHSO₂ (C₁-C₁₀ haloalkyl);
n^{111L} is selected from 1, 2, 3, 4 or 5.

Item 9, the compound of any one of items 1-8, wherein the spacer is a alkylene of 1 to 10 carbon atoms, wherein one or more carbon atoms are optionally replaced with any one or more substituent of the group consisting of: C(O), NH, O, S, OP(O)O, OP(S)O, CH═N and S(O)₂, and wherein the spacer is optionally not substituted or substituted by at least one group selected from group: H, or, C₁-C₅ alkyl, —OC₁-C₅ alkyl.

Item 10, the compound of item 7, wherein the branching group is selected from the group consisting of:

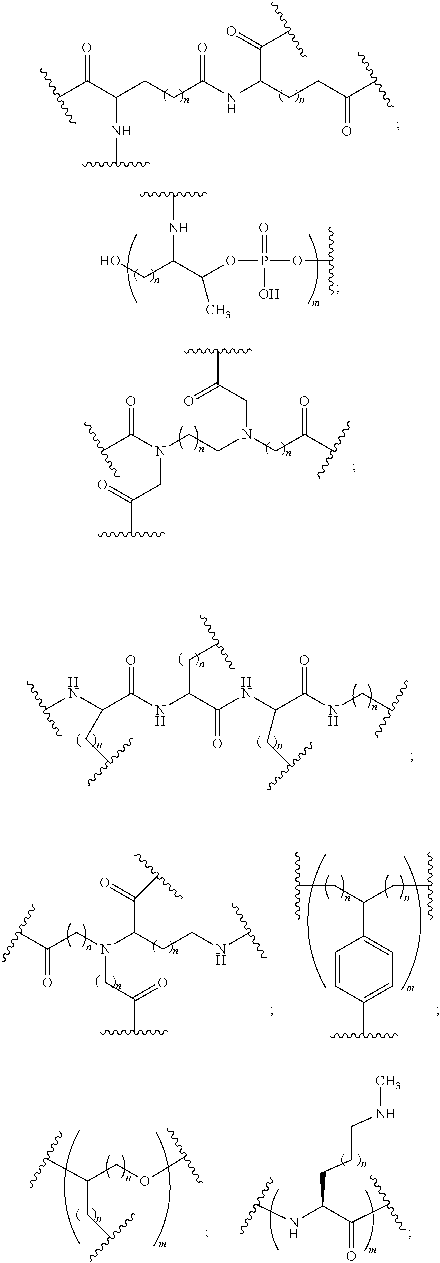

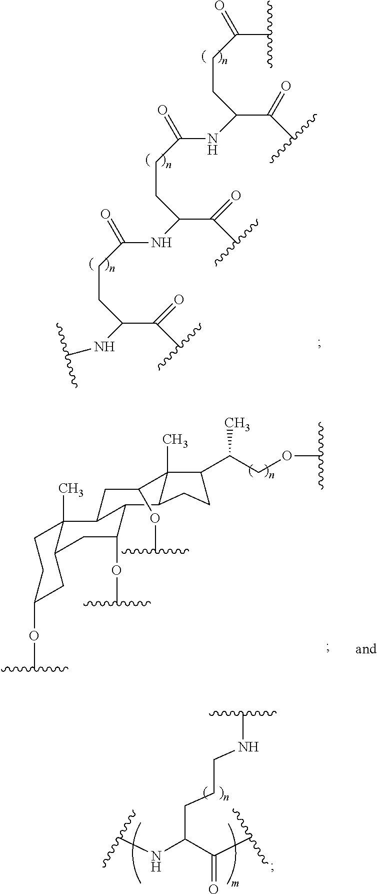

wherein each n is independently from 1 to 20; and
m is from 2 to 6.

Item 11, the compound of item 7, wherein the branching group is selected from group consisting of:

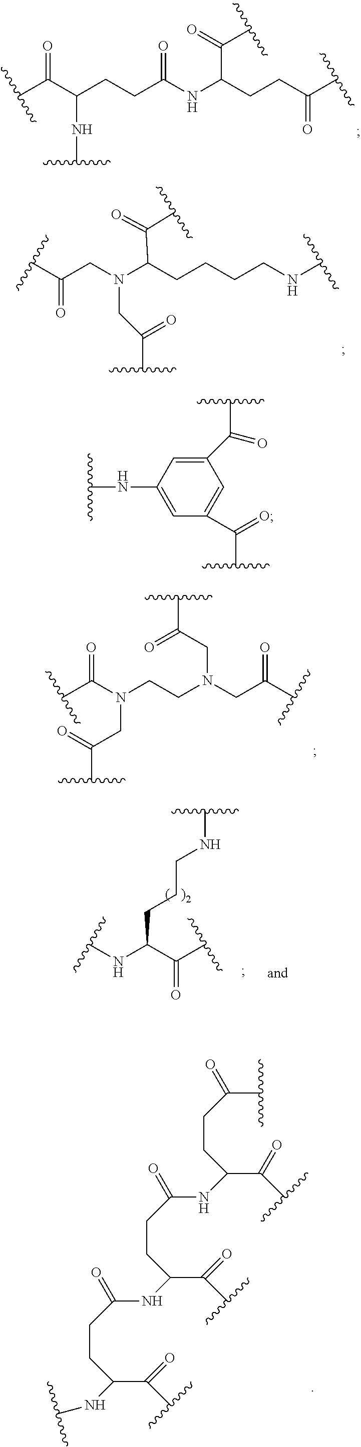

Item 12, the compound of item 7, wherein the branching group is selected from group consisting of:

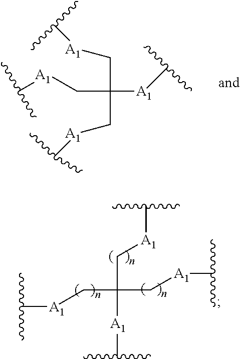

Wherein each A₁ is independently, O, S, C═O, or NH; and
each n is independently from 1 to 20.

Item 13, the compound of item 7, wherein the branching group is selected from group consisting of:

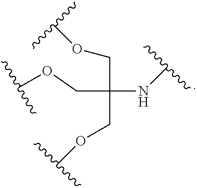 [SMILES: *NC(CO*)(CO*)CO*]

Item 14, the compound of item 1, wherein the compound having the structural formula

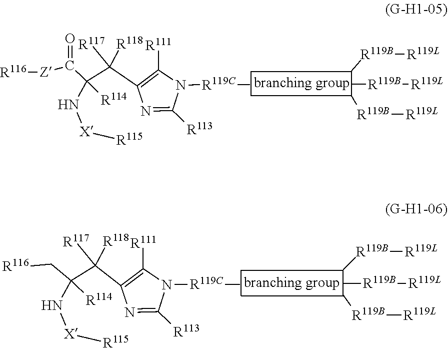

Wherein:
each X′ is independently selected from Table 1; each Z′ is independently selected from Table 2;

TABLE 2

| | |
|---|---|
| 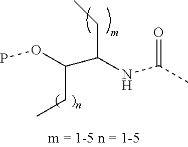 | 4-1 |
| 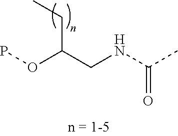 | 4-2 |
| 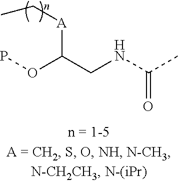 | 4-3 |
| 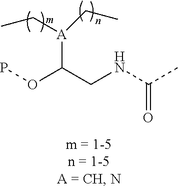 | 4-4 |
| 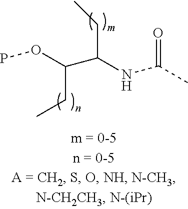 | 4-5 |
| 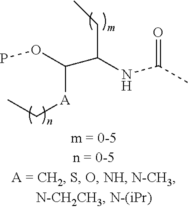 | 4-6 |
| 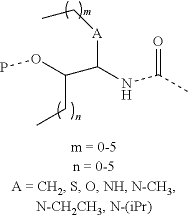 | 4-7 |
| 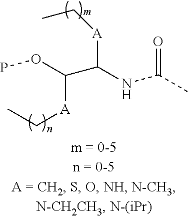 | 4-8 |
| 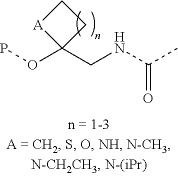 | 4-9 |
| 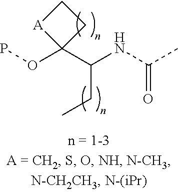 | 4-10 |
| 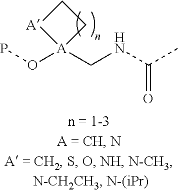 | 4-11 |
| 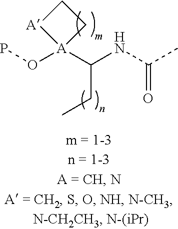 | 4-12 |
| 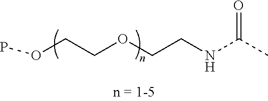 | 4-13 |
| 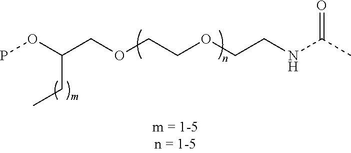 | 4-14 |
| 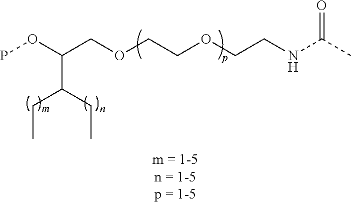 | 4-15 |
| 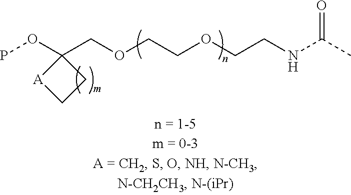 | 4-16 |
| 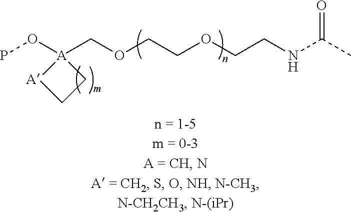 | 4-17 |
| 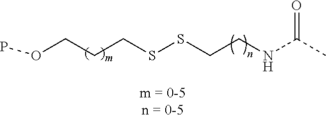 | 4-18 |
| 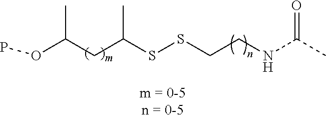 | 4-19 |
| 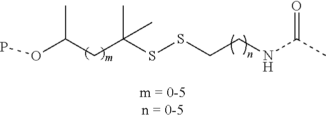 | 4-20 |

TABLE 1

| | |
|---|---|
| 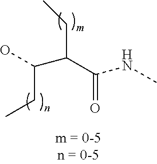 | 7-1 |
| 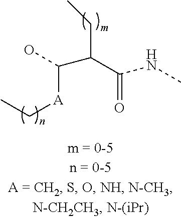 | 7-2 |
| 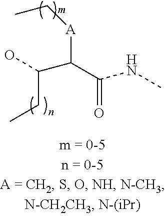 | 7-3 |
| 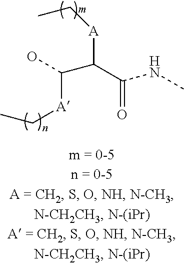 | 7-4 |
| 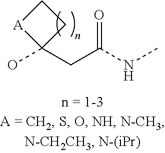 | 7-5 |
| 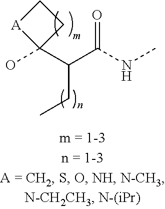 | 7-6 |
| 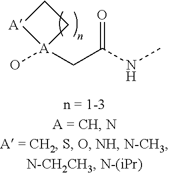 | 7-7 |
| 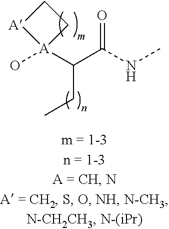 | 7-8 |
| 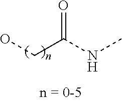 | 7-9 |
| 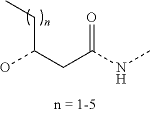 | 7-10 |
| 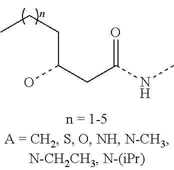 | 7-11 |
| 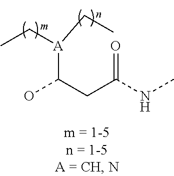 | 7-12 |
| 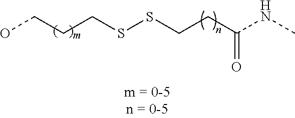 | 7-13 |
| 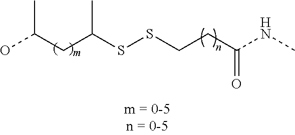 | 7-14 |
| 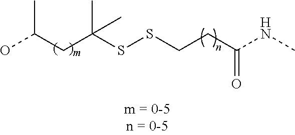 | 7-15 |
| 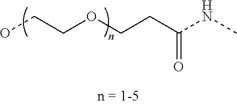 | 7-16 |
| 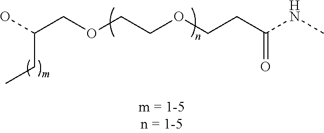 | 7-17 |
| 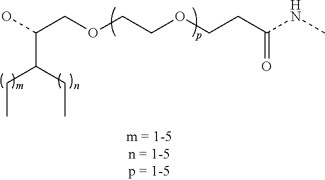 | 7-18 |
| 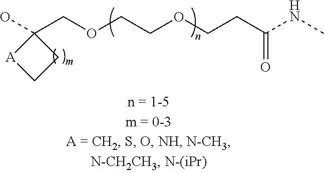 | 7-19 |
| 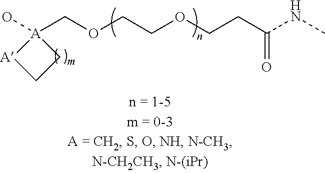 | 7-20 |

R^{119C} is selected from —C(O)—C₅-C₈ straight alkylene-NHCO—CH₂— or —C(O)—C₈-C₁₁ straight alkylene-.

Item 15, the compound of item 14, wherein the compound has the structural formula (G-H1-07) or (G-H1-08):

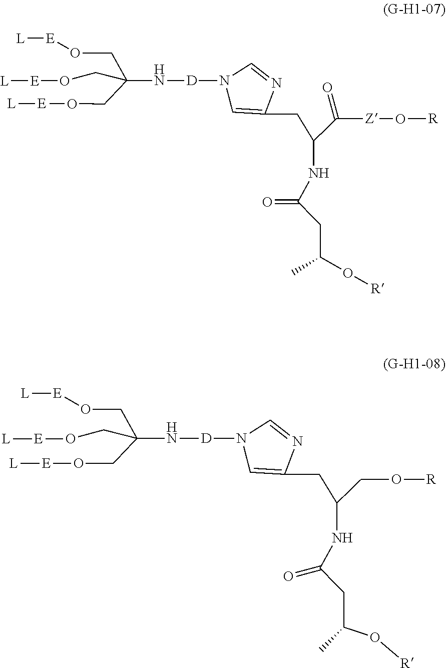

wherein:
R, R′ is independently selected from the group consisting of a solid support, an oligonucleotide comprising natural or chemically modified nucleotides/nucleosides, H and a protecting group;
at least one of R and R′ comprises an oligonucleotide formed by natural and/or chemically modified nucleotides/nucleosides;
D is selected from Table 3;
each E is independently selected from Table 4;

TABLE 3

| | |
|---|---|
| 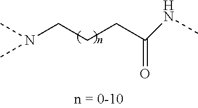 | 5-1 |
| 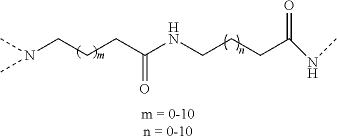 | 5-2 |
| 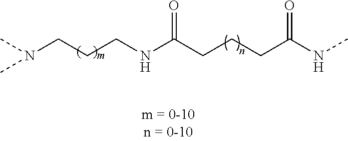 | 5-3 |
| 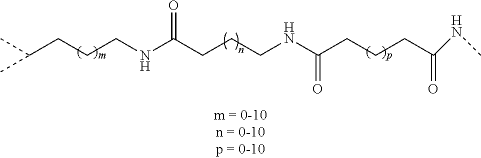 | 5-4 |
| 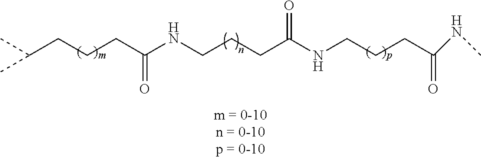 | 5-5 |
| 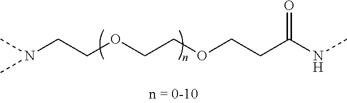 | 5-6 |
| 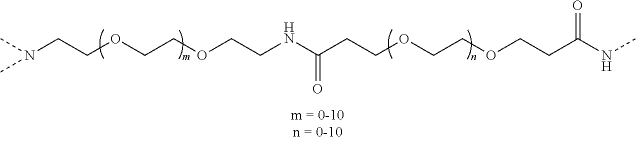 | 5-7 |
| 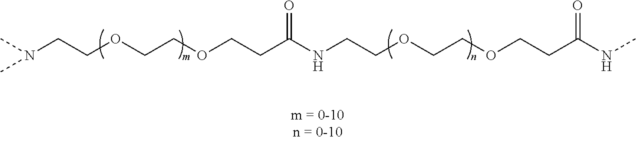 | 5-8 |
| 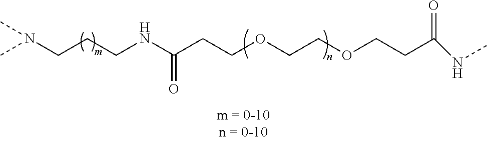 | 5-9 |
| 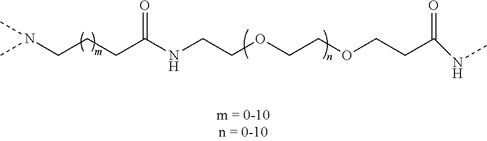 | 5-10 |
| 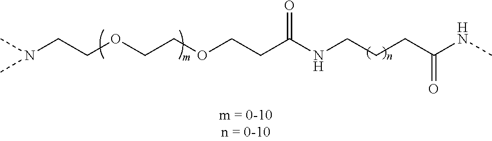 | 5-11 |
| 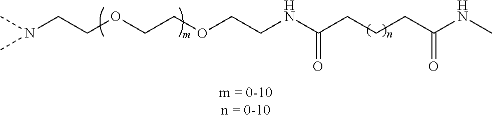 | 5-12 |
| 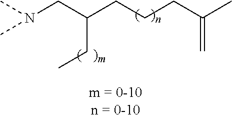 | 5-13 |

Z′ is independently selected from Table 2; and
each L independently comprises a ligand moiety capable of docking to a cell-surface receptor.

TABLE 4

| | |
|---|---|
| 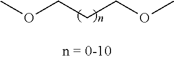 | 6-1 |
| 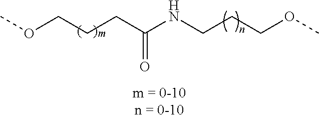 | 6-2 |
| 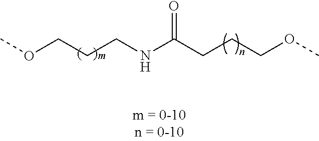 | 6-3 |
| 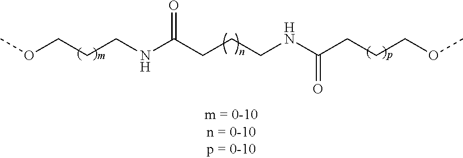 | 6-4 |
| 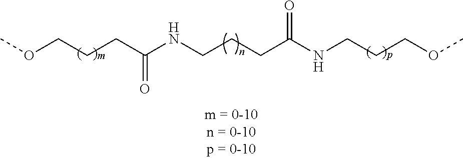 | 6-5 |
| 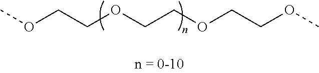 | 6-6 |
| 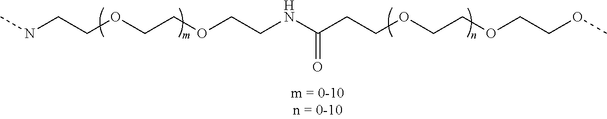 | 6-7 |
| 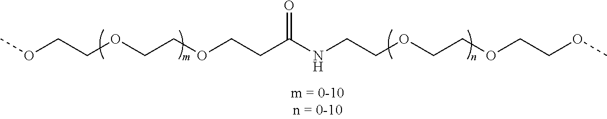 | 6-8 |
| 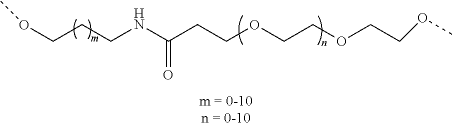 | 6-9 |
| 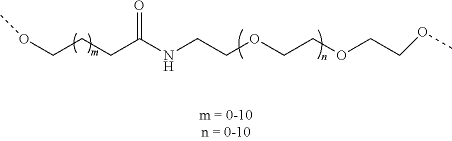 | 6-10 |
| 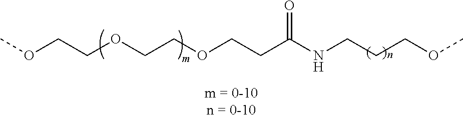 | 6-11 |
| 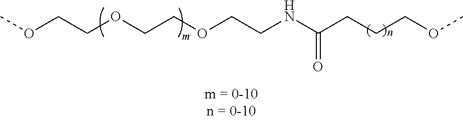 | 6-12 |
| 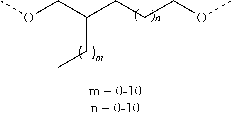 | 6-13 |

Item 16, the compound of item 15, wherein D is selected from —C(O)—C₅-C₈ straight alkylene-NHCO—CH₂— or —C(O)—C₈-C₁₁ straight alkylene-.

Item 17, the compound of item 14, wherein the compound has the structural formula (G-H1-09) or (G-H1-10):

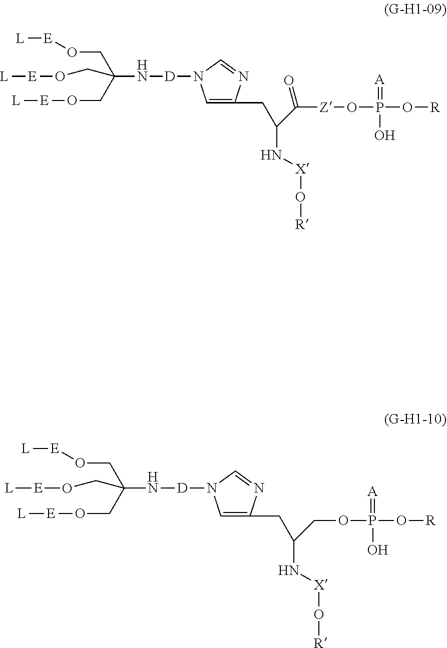

wherein:
R, R′ is independently selected from the group consisting of an oligonucleotide comprising natural or chemically modified nucleotides/nucleosides, H and a protecting group;
at least one of R and R′ comprises an oligonucleotide formed by natural and/or chemically modified nucleotides/nucleosides;
A independently is O or S;
X′ is independently selected from Table 1;
Z′ is independently selected from Table 2;
each D is selected from Table 3;
each E is independently selected from Table 4; and
each L independently comprises a ligand moiety capable of docking to a cell-surface receptor.

Item 18, the compound of item 17, wherein A is O.

Item 19, the compound of item 17, wherein A is S.

Item 20, the compound of any one of items 1-19, wherein each ligand is independently selected from the group consisting of N-acetyl galactosamine (GalNAc), cholesterol, tocopherol, biotin, cyanine dyes, folic acid, RGDp, transferrin, anisamide, lactobionic acid, cRGD, hyaluronic acid, low molecular weight protamine, lipid derivatives, peptides, cyclic peptides, and heterocycles.

Item 21, the compound of any one of items 1-20, wherein having the structural formula (G-H1-11)

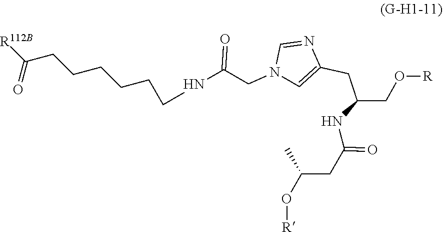

Wherein:
R^{112B} has the structure shown below:
-branching group-(R^{119B}-R^{119L})ₙ^{111L};
R^{119L} is independently selected from a ligand capable of docking to a cell surface receptor;
R^{119B} are independently sleeted from a alkylene of 1 to 30 carbon atoms, wherein one or more carbon atoms are optionally replaced with any one or more substituent of the group consisting of: C(O), NH, O, S, OP(O)O, OP(S)O, CH═N, S(O)₂, C₂-C₁₀ alkenylene, C₂-C₁₀ alkynylene, C₆-C₁₀ arylene, C₃-C₁₈ heterocyclylene, and C₅-C₁₀ heteroarylene, and wherein R^{119B} is optionally not substituted or substituted by R^{119C};
R¹¹⁴, R¹¹⁷, R¹¹⁸, R^{119C} are selected one or more from the group consisting of H, or, C₁-C₁₀ alkyl, C₆-C₁₀ aryl, C₅-C₁₀ heteroaryl, C₁-C₁₀ haloalkyl, —OC₁-C₁₀ alkyl, —OC₁-C₁₀ alkylphenyl, —C₁-C₁₀ alkyl-OH, —OC₁-C₁₀ haloalkyl, —SC₁-C₁₀ alkyl, —SC₁-C₁₀ alkylphenyl, —C₁-C₁₀ alkyl-SH, —SC₁-C₁₀ haloalkyl, halo, —OH, —SH, —NH₂, —C₁-C₁₀ alkyl-NH₂, —N(C₁-C₁₀ alkyl) (C₁-C₁₀ alkyl), —NH(C₁-C₁₀ alkyl), —N(C₁-C₁₀ alkyl)(C₁-C₁₀ alkylphenyl), —NH(C₁-C₁₀ alkylphenyl), cyano, nitro, —CO₂H, —C(O)OC₁-C₁₀ alkyl, —CON(C₁-C₁₀ alkyl) (C₁-C₁₀ alkyl), —CONH(C₁-C₁₀ alkyl), —CONH₂, —NHC(O)(C₁-C₁₀ alkyl), —NHC(O) (phenyl), —N(C₁-C₁₀ alkyl) C(O)(C₁-C₁₀ alkyl), —N(C₁-C₁₀ alkyl) C(O) (phenyl), —C(O) C₁-C₁₀ alkyl, —C(O)C₁-C₁₀ alkylphenyl, —C(O) C₁-C₁₀ haloalkyl, —OC(O)C₁-C₁₀ alkyl, —SO₂ (C₁-C₁₀ alkyl), —SO₂ (phenyl), —SO₂(C₁-C₁₀ haloalkyl), —SO₂NH₂, —SO₂NH(C₁-C₁₀ alkyl), —SO₂NH (phenyl), —NHSO₂(C₁-C₁₀ alkyl), —NHSO₂ (phenyl), and —NHSO₂(C₁-C₁₀ haloalkyl);
R, R′ is independently selected from the group consisting of a solid support, an oligonucleotide comprising natural or chemically modified nucleotides/nucleosides, H and a protecting group;
at least one of R and R′ comprises an oligonucleotide formed by natural and/or chemically modified nucleotides/nucleosides;
n^{111L} is selected from 1, 2, 3, 4 or 5.

Item 22, the compound of item 4, having the structural formula HC-1 to HC-8:

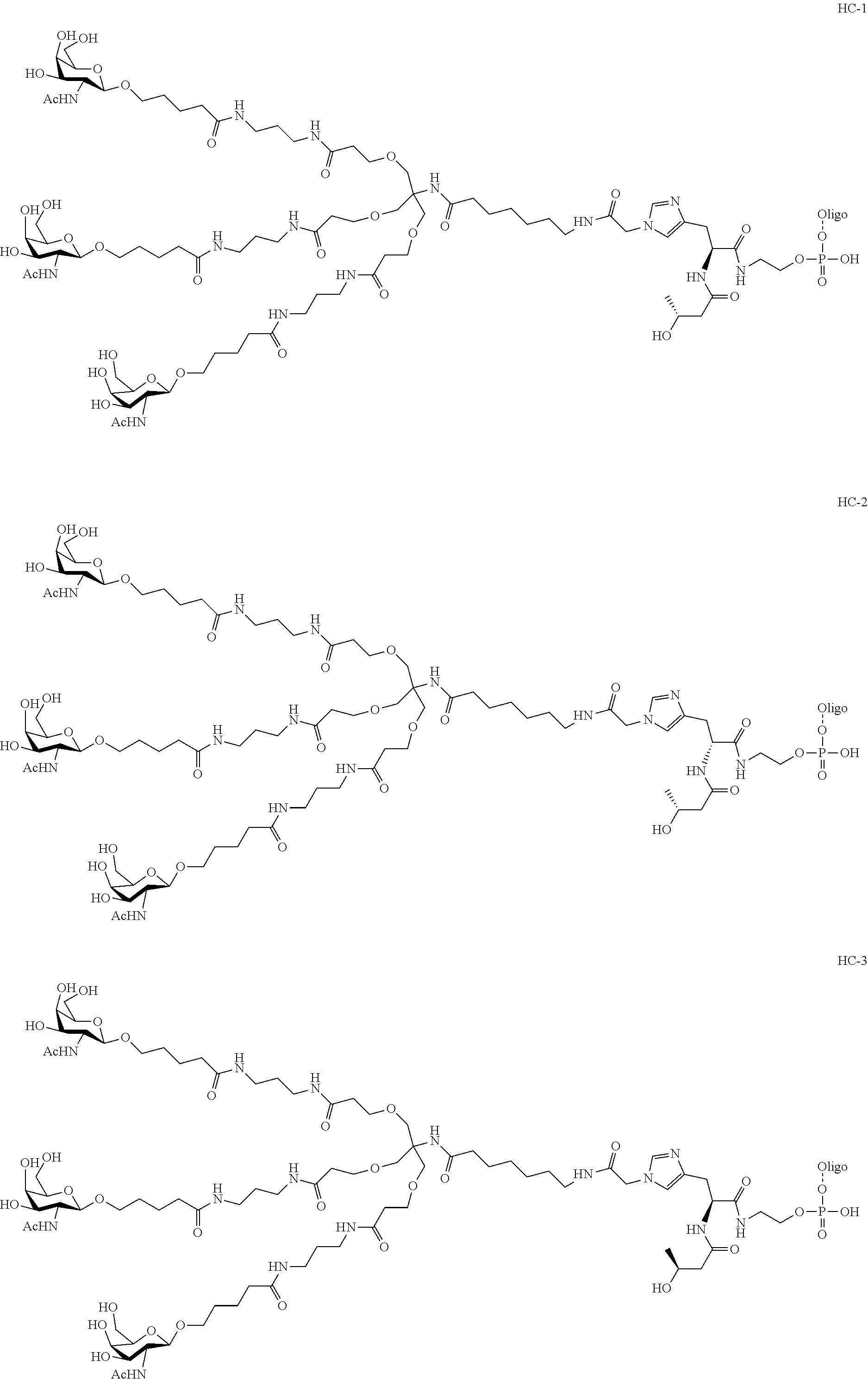

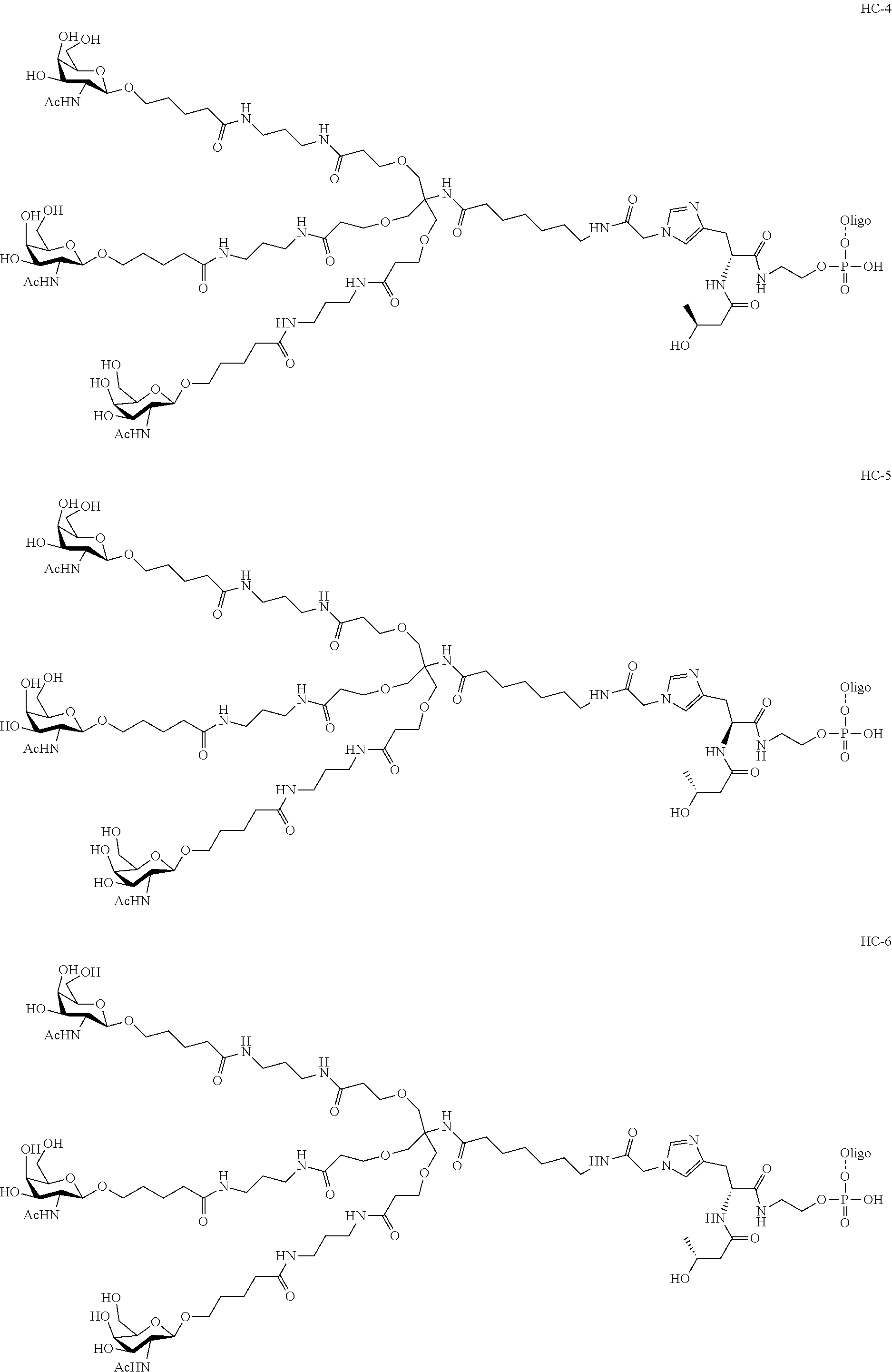

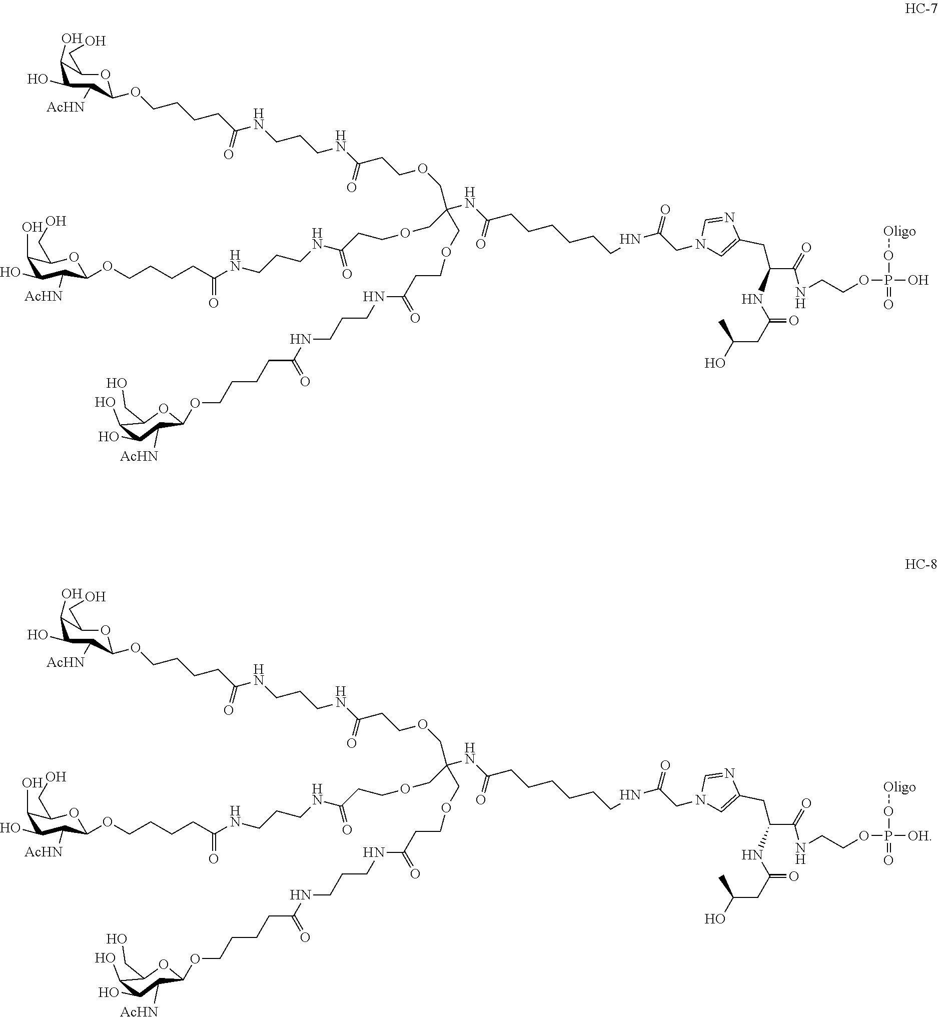

Item 23, the compound of item 4, having the structural formula HC-9:

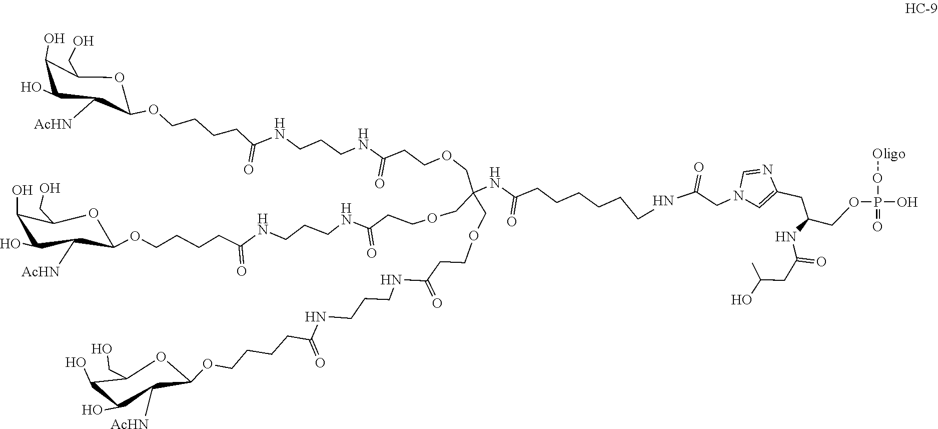

Item 24, the compound of item 4, having the structural formula HC-5:

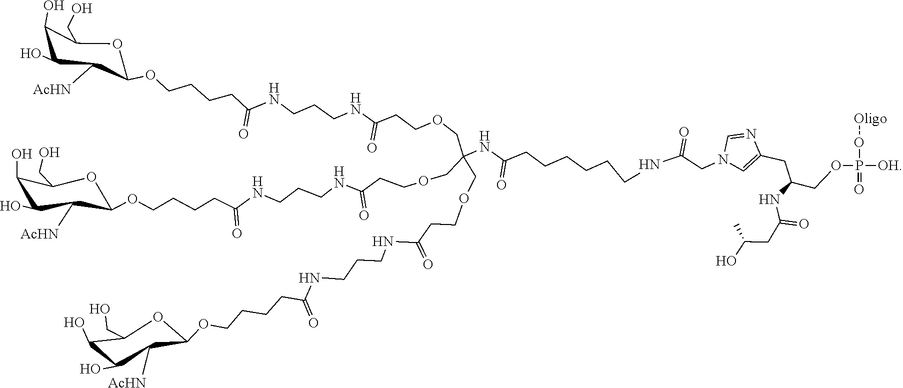

Item 25, the compound of any one of items 1-24, wherein the oligonucleotide is linked to the rest of the compound through its 5′ end and/or 3′ end.

Item 26, the compound of item 25, wherein the oligonucleotide comprises a small interfering RNA (siRNA) duplex.

Item 27, the compound of item 25, wherein the oligonucleotide comprises an asymmetric interfering RNA (aiRNA) duplex.

Item 28, the compound of item 27, wherein the aiRNA comprising an antisense strand and a sense strand, wherein the antisense strand is longer than the sense strand, has a length of 19, 20, 21, 22, 23, 24, 25, 26 or 27 nucleotides and includes a 3′-overhang of 1-9 nucleotides and a 5′-overhang of 0-8 nucleotides when duplexed with the sense strand; wherein the sense strand has a length of 12, 13, 14, 15, 16, 17, 18, 19, 20, 21, 22, 23, 24, 25 or 26 nucleotides and forms a double-stranded region with the antisense strand.

Item 29, the compound of item 28, wherein the aiRNA comprising an antisense strand and a sense strand, wherein the antisense strand is longer than the sense strand, has a length of 19, 20, 21, 22, 23, 24, 25, 26 or 27 nucleotides and includes a 3′-overhang of 1-9 nucleotides and a 5′-overhang of 1-8 nucleotides when duplexed with the sense strand; wherein the sense strand has a length of 12, 13, 14, 15, 16, 17, 18, 19, 20, 21, 22, 23, 24, 25 or 26 nucleotides and forms a double-stranded region with the antisense strand.

Item 30, the compound of item 28, wherein the aiRNA comprising an antisense strand and a sense strand, wherein the antisense strand is longer than the sense strand, has a length of 19, 20, 21, 22, 23, 24, 25, 26 or 27 nucleotides and includes a 3′-overhang of 1-9 nucleotides and a 5′ blunt end when duplexed with the sense strand; wherein the sense strand has a length of 12, 13, 14, 15, 16, 17, 18, 19, 20, 21, 22, 23, 24, 25 or 26 nucleotides and forms a double-stranded region with the antisense strand.

Item 31, the compound of item 25, wherein the oligonucleotide comprises an antisense oligonucleotide (ASO).

Item 32 the compound of item 25, wherein the oligonucleotide comprises micro-RNA (miRNA).

Item 33, a compound having the structural formula (G-G1):

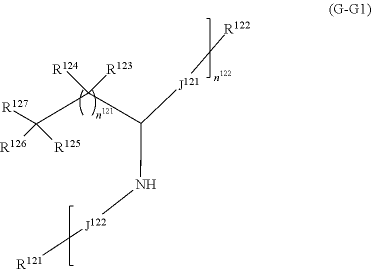

wherein:
R¹²⁷ comprises at least one ligand capable of docking to a cell surface receptor;
R¹²³, R¹²⁴, R¹²⁵, R¹²⁶ are selected one or more from the group consisting of H, alkyl, aryl, heteroaryl, haloalkyl, —O alkyl, —O alkylphenyl, -alkyl-OH, —O haloalkyl, —S alkyl, —S alkylphenyl, -alkyl-SH, —S haloalkyl, halo, —OH, —SH, —NH₂, -alkyl-NH₂, —N(alkyl) (alkyl), —NH (alkyl), —N (alkyl) (alkylphenyl), —NH (alkylphenyl), cyano, nitro, —CO₂H, —C(O)O alkyl, —CON (alkyl) (alkyl), —CONH (alkyl), —CONH₂, —NHC(O) (alkyl), —NHC(O) (phenyl), —N(alkyl) C(O) (alkyl), —N(alkyl) C(O) (phenyl), —C(O) alkyl, —C(O) alkylphenyl, —C(O) haloalkyl, —OC(O) alkyl, —SO₂ (alkyl), —SO₂ (phenyl), —SO₂ (haloalkyl), —SO₂NH₂, —SO₂NH (alkyl), —SO₂NH (phenyl), —NHSO₂ (alkyl), —NHSO₂ (phenyl), and —NHSO₂ (haloalkyl);
R¹²¹, R¹²² are each independently for each occurrence OH, a protecting group for OH, a phosphate group, a phosphodiester group, an activated phosphate group, an activated phosphite group, a phosphoramidite, a solid support, —OP(Z′)(Z″)O-nucleoside, —OP(Z′)(Z″)O-oligonucleotide, a lipid, a PEG, a steroid, a polymer, —O-nucleotide, a nucleoside, —OP(Z′)(Z″)O—R^{128B}—OP(Z′″)(Z″″)O-oligonucleotide, or an oligonucleotide;
J¹²¹, J¹²², R^{128B} are each independently for each occurrence a spacer;
Z′, Z″, Z′″ and Z″″ are each independently for each occurrence O or S;
n¹²¹, n¹²² are each independently for each occurrence 1, 2, 3, 4, 5 or 6;
the oligonucleotide comprising natural or chemically modified nucleotides/nucleosides.

Item 34, the compound of item 33, wherein the compound having the structural formula (G-G1-01):

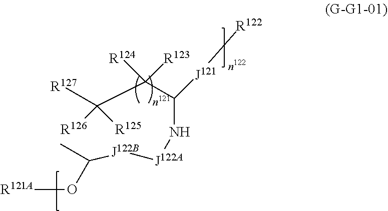

J^{122A} is selected from the group consisting of: C(O), NH, O, S, OP(O)O, OP(S)O, CH═N and S(O)₂;
J^{122B} is selected from a alkylene of 1 to 10 carbon atoms;
R^{121A} is selected from the group consisting of: H and a solid support;
R¹²² is selected from the group consisting of: OH, a protecting group for OH, a phosphate group, a phosphodiester group, an activated phosphate group, an activated phosphite group, a phosphoramidite, a solid support, —OP(Z′)(Z″)O-nucleoside, —OP(Z′)(Z″)O-oligonucleotide, a lipid, a PEG, a steroid, a polymer, —O-nucleotide, a nucleoside, —OP(Z′)(Z″)O—R^{128B}—OP(Z′″)(Z″″)O-oligonucleotide, or an oligonucleotide;
optionally, J^{122B} is selected from a straight alkylene of 1 to 10 carbon atoms.

Item 35, the compound of item 33 or item 34, wherein n¹²² is 1.

Item 36, the compound of any one of items 33-35, R¹²² comprises an oligonucleotide.

Item 37, the compound of item 36, wherein the compound having the structural formula (G-G1-02):

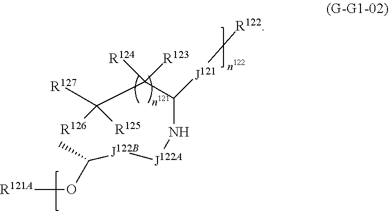

Item 38, the compound of item 36, wherein the compound having the structural formula (G-G1-03):

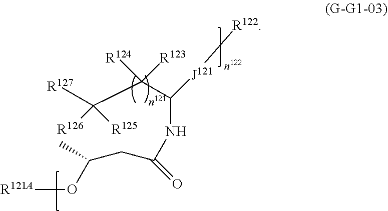

Item 39, the compound of item 36, wherein the compound having the structural formula (G-G1-04):

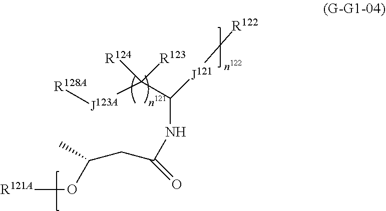

Wherein:
J^{123A} is selected from the group consisting of: C(O), NH, O, S, OP(O)O, OP(S)O, CH═N, and S(O)₂,
R^{128A} has the structure shown below:
R^{128C}-branching group-(R^{128B}-R^{128L})ₙ^{121L} or —R^{128B}-R^{128L};
R^{128L} is independently selected from a ligand capable of docking to a cell surface receptor;
R^{128B} are independently sleeted from a alkylene of 1 to 30 carbon atoms, wherein one or more carbon atoms are optionally replaced with any one or more substituent of the group consisting of: C(O), NH, O, S, OP(O)O, OP(S)O, CH═N, S(O)₂, C₂-C₁₀ alkenylene, C₂-C₁₀ alkynylene, C₆-C₁₀ arylene, C₃-C₁₈ heterocyclylene, and C₅-C₁₀ heteroarylene, and wherein R^{128B} is optionally not substituted or substituted by R^{128C};
R^{128C} is selected from —C(O)—C₅-C₈ straight alkylene-NHCO—CH₂— or —C(O)—C₈-C₁₁ straight alkylene-.
n^{121L} is selected from 1, 2, 3, 4 or 5;
R^{128L} is independently selected from a ligand capable of docking to a cell surface receptor;
n^{111L} is selected from 1, 2, 3, 4 or 5;
n¹²¹ is 2;
optionally, R^{128C} is selected from —C(O)—C₅-C₈ straight alkylene-NHCO—CH₂— or —C(O)—C₈-C₁₁ straight alkylene-.

Item 40, the compound of any one of items 33-39, wherein the spacer is a alkylene of 1 to 10 carbon atoms, wherein one or more carbon atoms are optionally replaced with any one or more substituent of the group consisting of: C(O), NH, O, S, OP(O)O, OP(S)O, CH═N and S(O)₂, and wherein the saper is optionally not substituted or substituted by at least one group selected from group: H, or, C₁-C₅ alkyl, —OC₁-C₅ alkyl.

Item 41, the compound of item 40, wherein the branching group is selected from group consisting of:

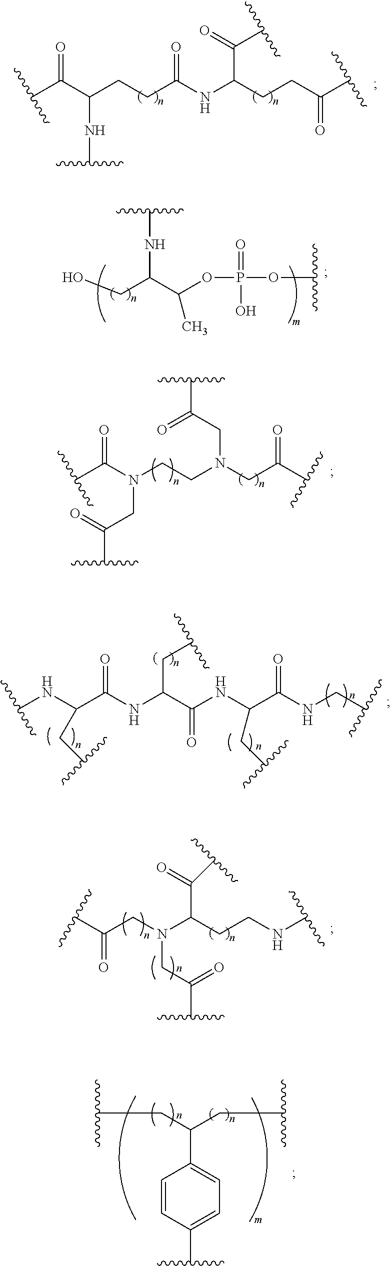

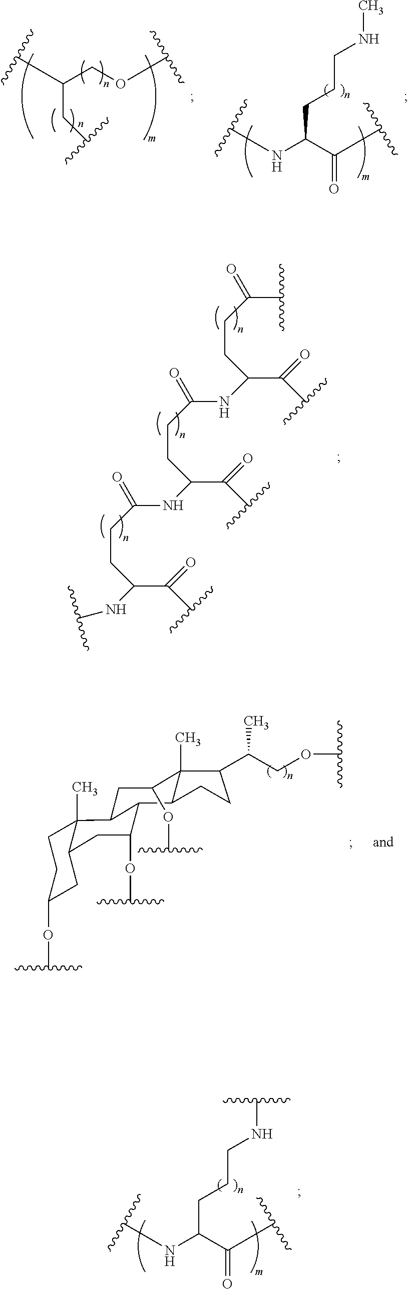

wherein each n is independently from 1 to 20; and
m is from 2 to 6.

Item 42, the compound of item 40, wherein the branching group is selected from the group consisting of:

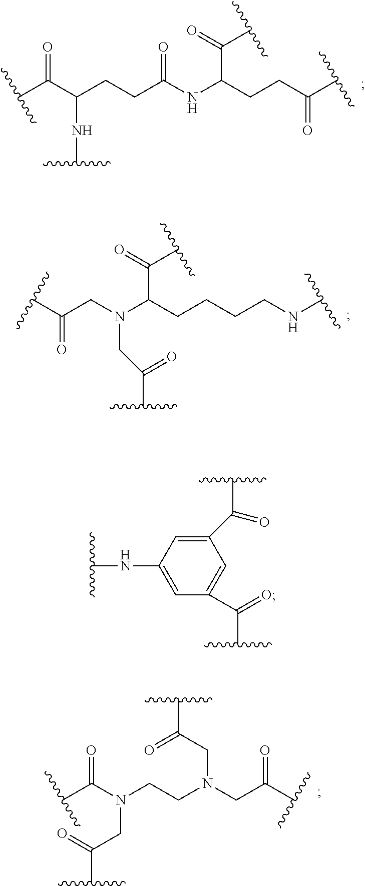 [SMILES: *NC(CCC(=O)NC(CCC(*)=O)C(*)=O)C(*)=O ; *NCCCCC(C(*)=O)N(CC(*)=O)CC(*)=O ; *Nc1cc(C(*)=O)cc(C(*)=O)c1 ; *C(=O)CN(CCN(CC(*)=O)C(*)=O)CC(*)=O]

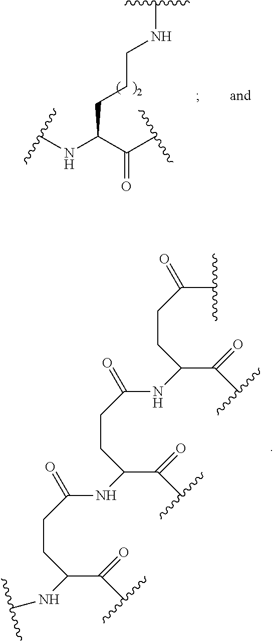

Item 43, the compound of item 40, wherein the branching group is selected from the group consisting of:

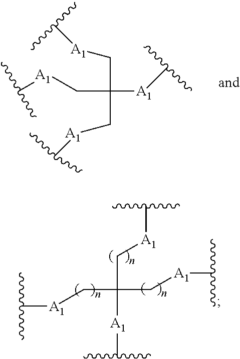

Wherein each A₁ is independently, O, S, C═O, or NH; and
each n is independently from 1 to 20.

Item 44, the compound of item 40, wherein the branching group is selected from group consisting of:

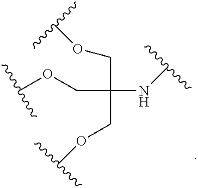 [SMILES: *NC(CO*)(CO*)CO*]

Item 45, the compound of item 33, wherein the compound having the structural formula (G-G1-05) or (G-G1-06):

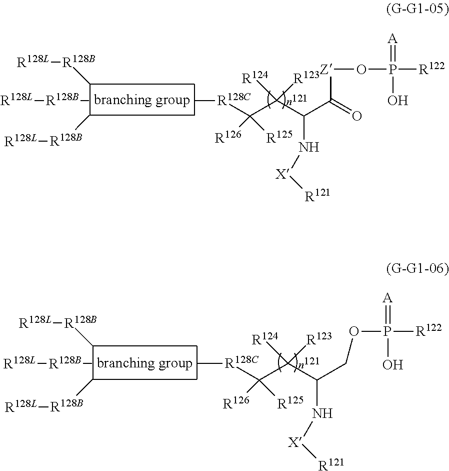

wherein
X′ is independently selected from Table 1; Z′ is independently selected from Table 2;
R^{128C} is selected from —C(O)—C₅-C₈ straight alkylene-NHCO—CH₂— or —C(O)—C₈-C₁₁ straight alkylene-.

Item 46, the compound of item 33, wherein the compound having the structural formula (G-G1-07) or (G-G1-08):

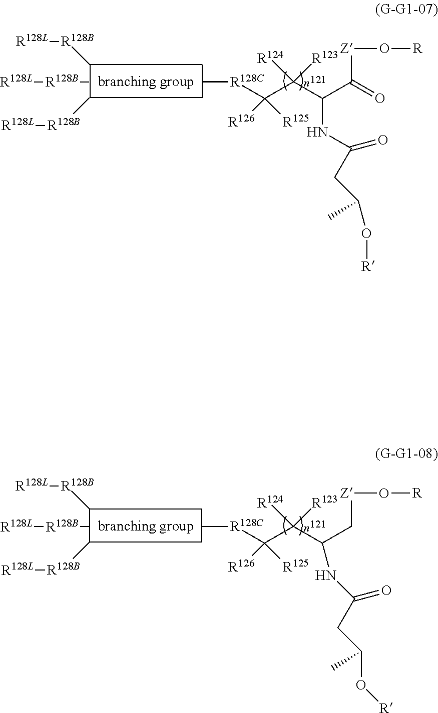

Wherein:
R, R′ is independently selected from an oligonucleotide comprising natural or chemically modified nucleotides/nucleosides, H and a protecting group;
at least one of R and R′ comprises an oligonucleotide formed by natural and/or chemically modified nucleotides/nucleosides;
Z′ is independently selected from Table 2;
R^{128C} is selected from —C(O)—C₅-C₈ straight alkylene-NHCO—CH₂— or —C(O)—C₈-C₁₁ straight alkylene-.

Item 47, the compound of item 33, wherein the compound has the structural formula (G-G1-09) or (G-G1-10):

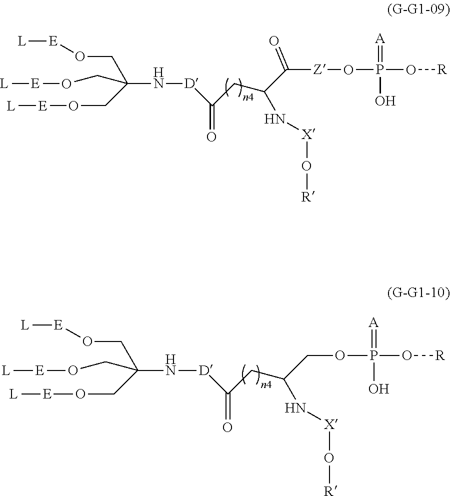

wherein:
R, R′ is independently selected from an oligonucleotide comprising natural or chemically modified nucleotides/nucleosides, H and a protecting group;
at least one of R and R′ comprises an oligonucleotide formed by natural and/or chemically modified nucleotides/nucleosides;
A is O or S;
D′ is selected from Table 9;
each E is selected from Table 4;
X′ is independently selected from Table 1;
Z′ is independently selected from Table 2.
each L independently comprises a ligand moiety capable of docking to a cell-surface receptor; and
n4 is independently selected from 1, 2, 3 and 4.

TABLE 9

| | |
|---|---|
| 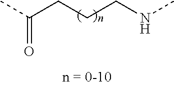 | 9-1 |
| 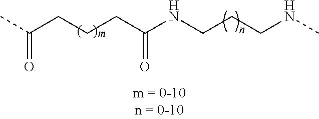 | 9-2 |
| 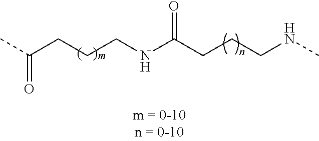 | 9-3 |
| 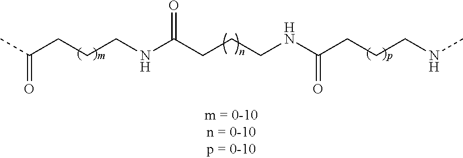 | 9-4 |
| 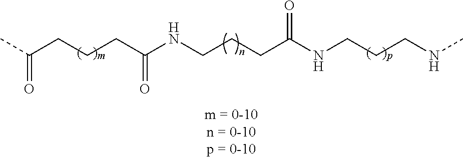 | 9-5 |
| 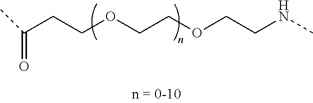 | 9-6 |
| 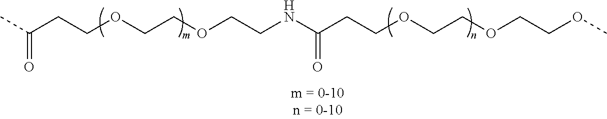 | 9-7 |
| 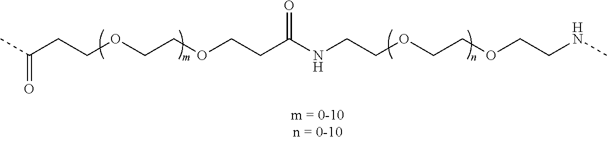 | 9-8 |
| 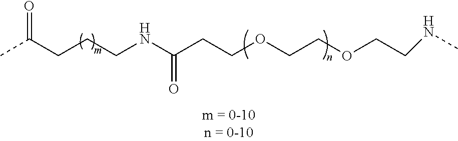 | 9-9 |
| 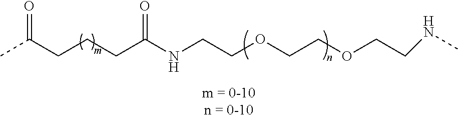 | 9-10 |
| 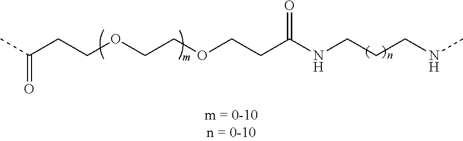 | 9-11 |
| 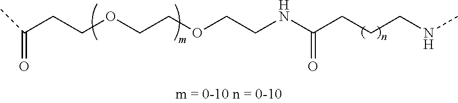 | 9-12 |
| 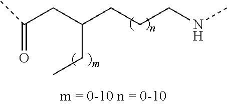 | 9-13 |

Item 48, the compound of item 47, wherein A is O.

Item 49, the compound of item 47, wherein A is S.

Item 50, the compound of any items 33-49, wherein each ligand is independently selected from the group consisting of N-acetyl galactosamine (GalNAc), cholesterol, tocopherol, biotin, cyanine dyes, folic acid, RGDp, transferrin, anisamide, lactobionic acid, cRGD, hyaluronic acid, low molecular weight protamine, lipid deriviatives, peptides, cyclic peptides, and heterocycles.

Item 51, the compound of item 33, wherein having the structural formula

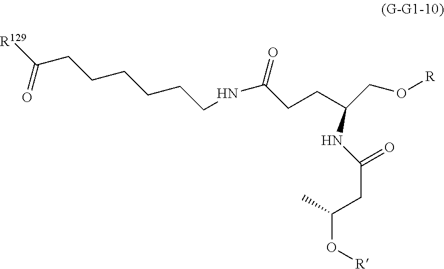

Wherein:
R¹²⁹ has the structure shown below:
-branching group-(R^{128B}-R^{128L})ₙ^{121L};
R^{128L} is independently selected from a ligand capable of docking to a cell surface receptor;
R^{128B} are independently sleeted from a alkylene of 1 to 30 carbon atoms, wherein one or more carbon atoms are optionally replaced with any one or more substituent of the group consisting of: C(O), NH, O, S, OP(O)O, OP(S)O, CH═N, S(O)₂, C₂-C₁₀ alkenylene, C₂-C₁₀ alkynylene, C₆-C₁₀ arylene, C₃-C₁₅ heterocyclylene, and C₅-C₁₀ heteroarylene, and wherein R^{128B} is optionally not substituted or substituted by R^{128C};
R, R′ is independently selected from the group consisting of a solid support, an oligonucleotide comprising natural or chemically modified nucleotides/nucleosides, H and a protecting group; at least one of R and R′ comprises an oligonucleotide formed by natural and/or chemically modified nucleotides/nucleosides;
n^{121L} is selected from 1, 2, 3, 4 or 5.

Item 52, the compound of item 36, wherein the compound has the structure shown in formula GC-1 to GC-8:

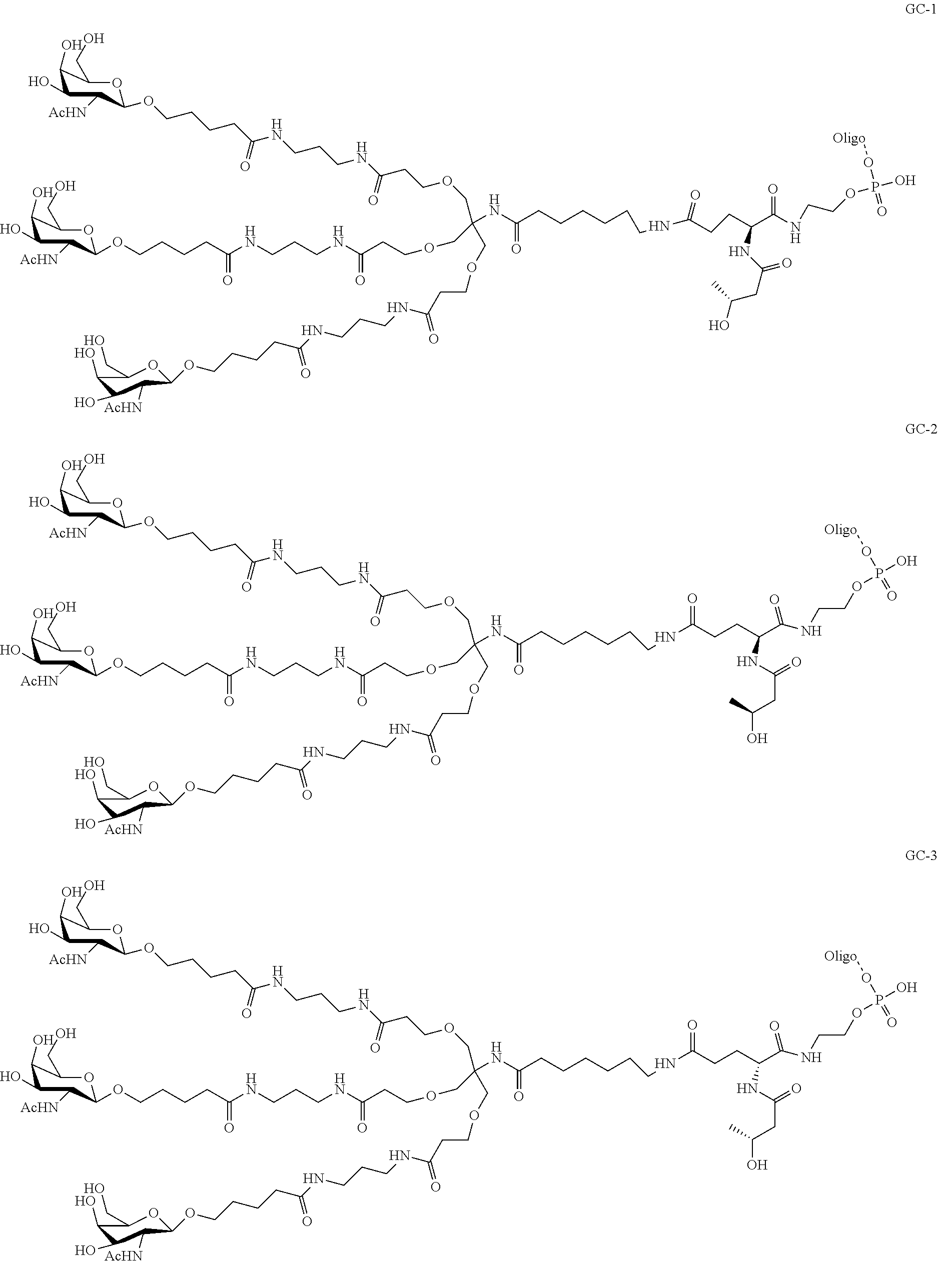

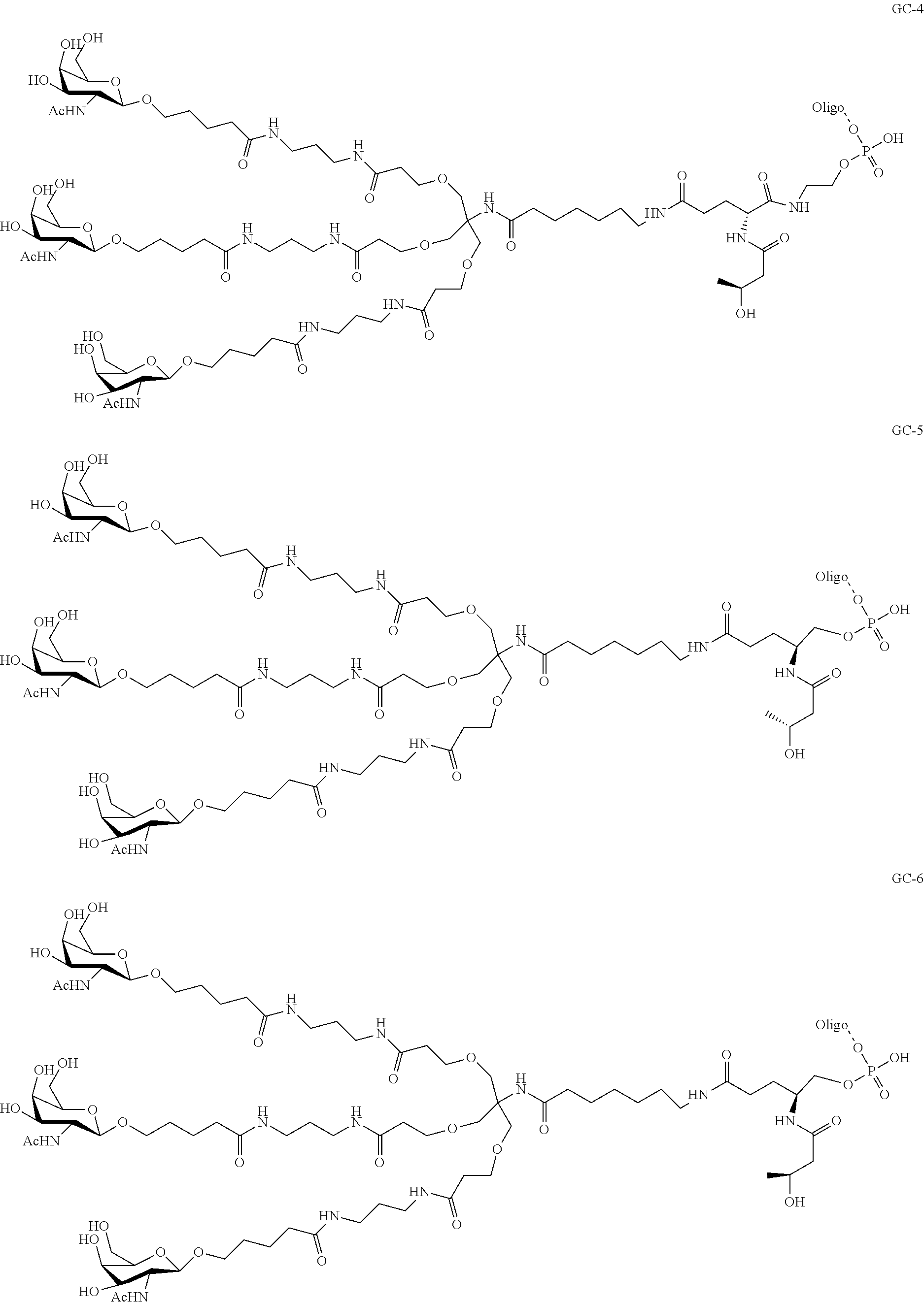

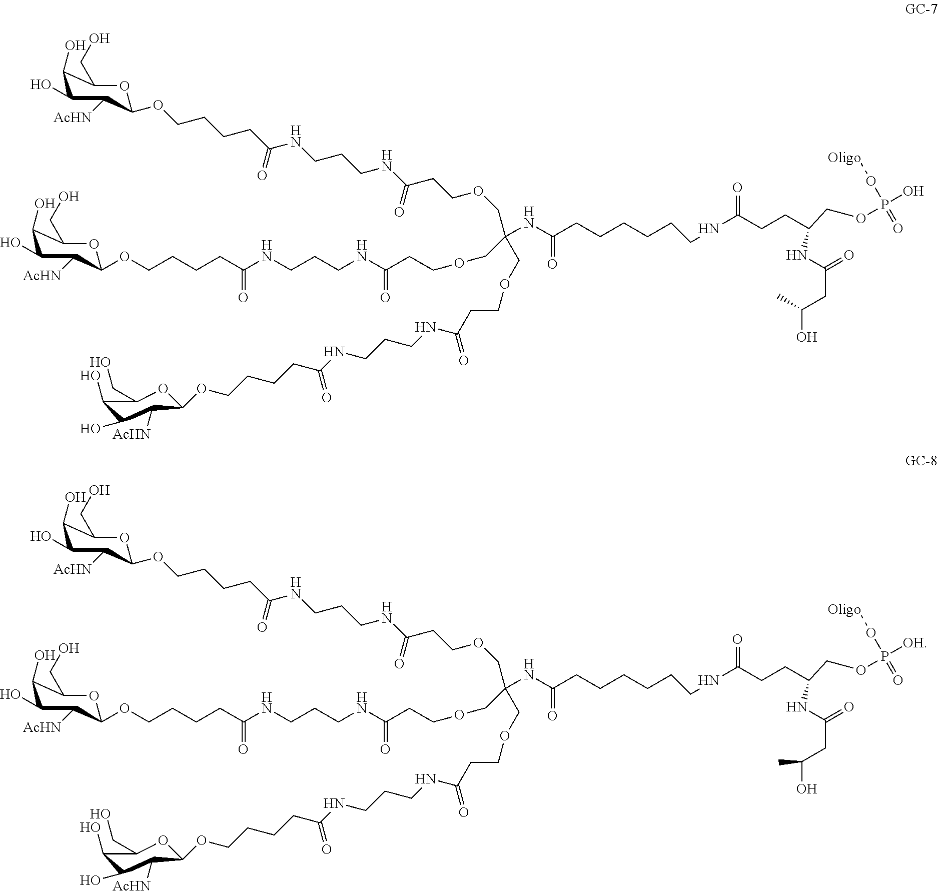

Item 53, the compound of item 36, having the structural formula GC-9

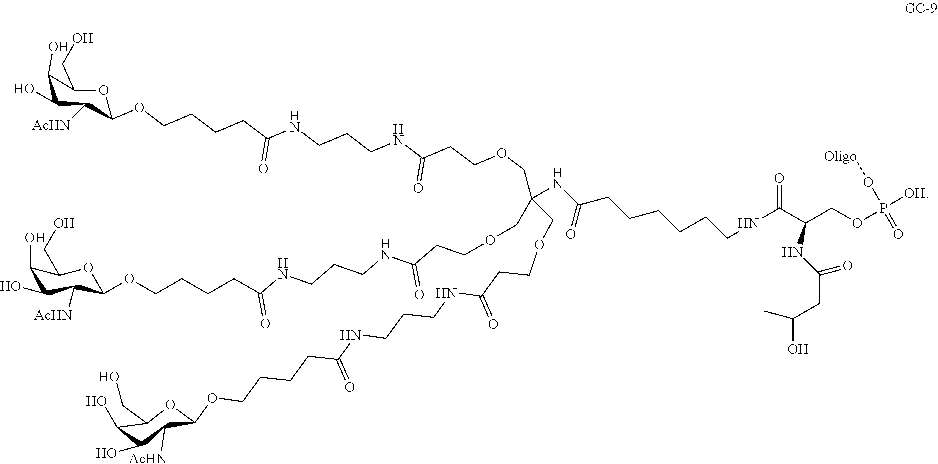

Item 54, the compound of item 36, having the structural formula GC-5

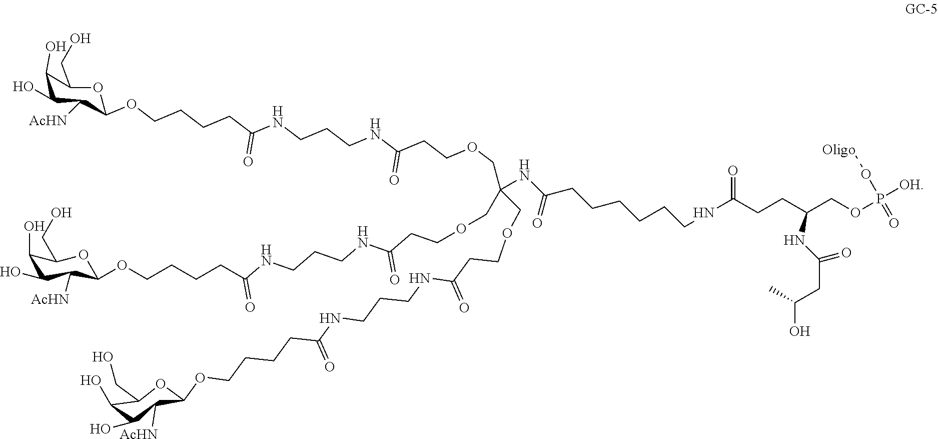

Item 55, the compound of any of items 33-54, wherein the oligonucleotide is linked to the rest of the compound through its 5′ end and/or 3′ end.

Item 56, a compound of item 55, wherein the oligonucleotide comprises a small interfering RNA (siRNA) duplex.

Item 57, a compound of any of items 33-54, wherein the oligonucleotide comprises an asymmetric interfering RNA (aiRNA) duplex.

Item 58, a compound of item 57, wherein the aiRNA comprising an antisense strand and a sense strand, wherein the antisense strand is longer than the sense strand, has a length of 19, 20, 21, 22, 23, 24, 25, 26 or 27 nucleotides and includes a 3′-overhang of 1-9 nucleotides and a 5′-overhang of 0-8 nucleotides when duplexed with the sense strand; wherein the sense strand has a length of 12, 13, 14, 15, 16, 17, 18, 19, 20, 21, 22, 23, 24, 25 or 26 nucleotides and forms a double-stranded region with the antisense strand.

Item 59, a compound of item 58, wherein the aiRNA comprising an antisense strand and a sense strand, wherein the antisense strand is longer than the sense strand, has a length of 19, 20, 21, 22, 23, 24, 25, 26 or 27 nucleotides and includes a 3′-overhang of 1-9 nucleotides and a 5′-overhang of 1-8 nucleotides when duplexed with the sense strand; wherein the sense strand has a length of 12, 13, 14, 15, 16, 17, 18, 19, 20, 21, 22, 23, 24, 25 or nucleotides and forms a double-stranded region with the antisense strand.

Item 60, a compound of item 58, wherein the aiRNA comprising an antisense strand and a sense strand, wherein the antisense strand is longer than the sense strand, has a length of 19, 20, 21, 22, 23, 24, 25, 26 or 27 nucleotides and includes a 3′-overhang of 1-9 nucleotides and a 5′ blunt end when duplexed with the sense strand; wherein the sense strand has a length of 12, 13, 14, 15, 16, 17, 18, 19, 20, 21, 22, 23, 24, 25 or 26 nucleotides and forms a double-stranded region with the antisense strand.

Item 61, a compound of any of items 33-54, wherein the oligonucleotide comprises an antisense oligonucleotide (ASO).

Item 62, a compound of any of items 33-54, wherein the oligonucleotide comprises micro-RNA (miRNA).

Item 63, a small interfering RNA (siRNA) agent comprising a structural formula of any of items 1-55.

Item 64, a asymmetric interfering RNA (aiRNA) agent comprising a structural formula of any of items 1-55.

Item 65, a antisense oligonucleotide (ASO) agent comprising a structural formula of any of items 1-55.

Item 66, a micro-RNA (miRNA) agent comprising a structural formula of any of items 1-55.

Item 68, a pharmaceutical composition comprising a compound of any one of items 1-60 or agent of any one of items 63-66 and a pharmaceutically acceptable excipient, carrier, or diluent.

Use of a compound of any one of items 1-60 or agent of any one of items 63-66 in preparation of a medicament effective for treating a disease or a condition.

In third aspect, the invention features a compound comprising a carbohydrate ligand as provided in the second aspect above, and the presence of the carbohydrate ligand can increase delivery of the compound to the targeted organs, e.g. liver. Thus, a compound comprising a carbohydrate ligand can be useful for targeting a gene related to a disease or an undesired condition in the targeted organs. For example, a compound of the invention comprising the carbohydrate ligand can target a nucleic acid expressed by a hepatitis virus. In other examples, the target gene can be selected from the group consisting of: Factor VII, Eg5, PCSK9, APOC3, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-I gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21(WAFl/CIPl) gene, mutations in the p27(KIPl) gene, mutations in the PPMlD gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene.

In a further aspect, the invention provides a pharmaceutical composition comprising a compound of the invention as provided in any aspects above and a pharmaceutically acceptable excipient, carrier, or diluent.

In another aspect, the invention features a method for delivering a compound to a specific target in a subject for therapeutic or diagnostic purpose. Accordingly, the invention provides a method for treating or preventing a disease or a condition, wherein the method comprises administering to a subject in need thereof an effective amount of a pharmaceutical composition that includes the compound of the invention. The treatment or prevention of a disease or condition is carried by partial or total silencing of disease genes. The disease genes may be patient's own genes or microbial genes come from outside, such as virus.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings illustrate the core idea of the invention but do not necessarily conclude all the possible variations. In the drawings, like numerals are used to indicate like parts throughout the various views.

FIG. 1 illustrates exemplary structures of Oligonucleotide-Ligand Conjugation. A conjugated interfering RNA duplex molecule comprises an antisense strand and a sense strand. In some embodiments, the oligonucleotide is interfering RNA duplex molecule, and the Ligand can be conjugated at the 3′ end of sense strand (such as Structure 1.1-1.3, middle type aiRNA, blunt end type aiRNA and siRNA), at the 3′ end of antisense strand (Structure 2), at the 5′ end of sense strand (Structure 3), or at both two ends of sense strand (Structure 5), at both two ends of antisense strand (Structure 4), at the 3′ end of antisense strand and 5′ end of sense strand (Structure 6), at the 3′ end of sense strand and 3′ end of antisense strand (Structure 7), or at the 3′ end of sense strand, 3′ end of antisense strand and the 5′ end of sense strand. In some embodiments, the oligonucleotide is antisense oligonucleotide (ASO), and the Ligand can be conjugated at the 3′ end or/and 5′ end of the antisense strand.

FIG. 2 illustrates the ex vivo uptake β-Catenin aiRNA results tested by QPCR. “Non-GalNAc” is non-conjugated aiRNA. “GalNAc” is aiRNA conjugated with “His-Cluster”.

FIG. 3 illustrates the ex vivo uptake potency of the mCat12 aiRNA conjugated with “His-cluster(3 GalNAc)”, and “Glu-cluster(3 GalNAc)” in primary hepatocytes.

FIG. 4 illustrates the uptake potency of the mCat12 aiRNA conjugated with “His-cluster(3 GalNAc)”, and “Glu-cluster(3 GalNAc)” in vivo. The aiRNA is administered at dose of 20 mg/Kg s.c.

FIG. 5 illustrates the uptake potency of the mCat12 aiRNA conjugated with “His-cluster(3 GalNAc)”, and “Glu-cluster(3 GalNAc)” in vivo. The aiRNA is administered at dose of 2 mg/Kg s.c.

FIG. 6 illustrates the ex vivo uptake β-Catenin aiRNA results tested by QPCR. aiRNA is conjugated with “His-Cluster”. SS-Middle represent aiRNA #1 with 5′ overhang on antisense strand. SS-3′Blunt represent aiRNA #2 that has blunt end at 3′ sense strand and 5′ antisense strand.

FIG. 7 illustrates the iv vivo uptake β-Catenin aiRNA results. SS-Middle represent aiRNA #1 with 5′ overhang and 3′ overhang on antisense strand. SS-3′Blunt represent aiRNA #2 that has blunt end at 3′ sense strand and 5′ antisense strand.

### DETAILED DESCRIPTION OF THE INVENTION

### I. Definition

Unless otherwise noted, technical terms are used according to conventional usages. Definitions of common terms in molecular biology may be found, for example, in J. Krebs et al. (eds.), Lewin's Genes XII, published by Jones and Bartlett Learning, 2017 (ISBN 9781284104493); Robert A. Meyers (ed.), Molecular Biology and Biotechnology: a Comprehensive Desk Reference, published by Anmol Publications Pvt. Ltd, 2011 (ISBN 9788126531783); and other similar technical references.

As used in the specification and claims, the singular form “a”, “an”, or “the” includes plural references unless the context clearly dictates otherwise. For example, the term “a cell” includes a plurality of cells including mixtures thereof. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent,” or “except for [a particular feature or element],” or “wherein [a particular feature or element] is not present (included, etc.) . . . ”.

When a dimensional measurement is given for a part herein, the value is, unless explicitly stated or clear from the context, meant to describe an average for a necessary portion of the part, i.e., an average for the portion of the part that is needed for the stated purpose. Any accessory or excessive portion is not meant to be included in the calculation of the value.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values >0 and <2 if the variable is inherently continuous.

As used herein, “about” means within plus or minus 10%. For example, “about 1” means “0.9 to 1.1”, “about 2%” means “1.8% to 2.2%”, “about 2% to 3%” means “1.8% to 3.3%”, and “about 3% to about 4%” means “2.7% to 4.4%.”

As used herein, the terms “spacer”, “linker” and “linkage” are used to link the two parts of the compounds, e.g. alkylene of 1 to 10 carbon atoms, alkylene of 1 to 10 carbon atoms which is one or more carbon atoms are optionally replaced with any one or more substituent of the group consisting of: C(O), NH, O, S, OP(O)O, OP(S)O, CH═N, S(O)₂, alkylene of 1 to 10 carbon atoms which is not substituted or substituted by at least one group selected from the group consisting of: H, C₁-C₅ alkyl, and —OC₁-C₅ alkyl.

Various hydroxyl protecting groups may be used in the present disclosure. In general, protecting groups render chemical functionalities inert to specific reaction conditions, and may be appended to and removed from such functionalities in a molecule without substantially damaging the remainder of the molecule. Representative hydroxyl protecting groups are disclosed by Beaucage, et al., Tetrahedron 1992, 48, 2223-2311, and also in Greene and Wuts, Protective Groups in Organic Synthesis, Chapter 2, 2d ed, John Wiley & Sons, New York, 1991, each of which are hereby incorporated by reference in their entirety. In some embodiments, the protecting group is stable under basic conditions but may be removed under acidic conditions. In some embodiments, non-exclusive examples of the hydroxyl protecting groups that may be used herein include dimethoxytrityl (DMT), monomethoxytrityl, 9-phenylxanthen-9-yl (Pixyl) and 9-(p-methoxyphenyl) xanthen-9-yl (Mox). In some embodiments, non-exclusive examples of the hydroxyl protecting groups that may be used herein comprises Tr (trityl), MMTr (4-methoxytrityl), DMTr (4,4′-dimethoxytrityl), and TMTr (4,4′,4″-trimethoxytrityl).

As used herein, a dash (“—”) that is not between two letters or symbols is used to indicate a point of attachment for a substituent. For example, —C₁-C₁₀ alkyl-NH₂ is attached through the C₁-C₁₀ alkyl.

As used herein, “optional” or “optionally” is meant that the subsequently described event or circumstance may or may not occur, and that the description includes instances wherein the event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, “alkyl” refers to straight chain and branched chain having the indicated number of carbon atoms, usually from 1 to 20 carbon atoms, for example 1 to 10 carbon atoms, such as 1 to 8 or 1 to 6 carbon atoms. For example, C₁-C₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes n-propyl and isopropyl. Alkylene is a subset of alkyl, referring to the same residues as alkyl, but having two points of attachment.

As used herein, “alkenyl” refers to an unsaturated branched or straight-chain alkyl group having at least one carbon-carbon double bond derived by the removal of one molecule of hydrogen from adjacent carbon atoms of the parent alkyl. The group may be in either the cis or trans configuration about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl; and the like. In certain embodiments, an alkenyl group has from 2 to 20 carbon atoms and in other embodiments, from 2 to 10, 2 to 8, or 2 to 6 carbon atoms. Alkenylene is a subset of alkenyl, referring to the same residues as alkenyl, but having two points of attachment.

As used herein, “alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 2 to 10, 2 to 8, or 2 to 6 carbon atoms. Alkynylene is a subset of alkynyl, referring to the same residues as alkynyl, but having two points of attachment.

As used herein, “alkoxy” refers to an alkyl group of the indicated number of carbon atoms attached through an oxygen bridge such as, for example, methoxy, ethoxy, propoxy, isopropoxy, n-butoxy, sec-butoxy, tert-butoxy, pentyloxy, 2-pentyloxy, isopentyloxy, neopentyloxy, hexyloxy, 2-hexyloxy, 3-hexyloxy, 3-methylpentyloxy, and the like. Alkoxy groups will usually have from 1 to 10, 1 to 8, 1 to 6, or 1 to 4 carbon atoms attached through the oxygen bridge.

As used herein, “aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl, fluorenyl, and naphthyl. Arylene is a subset of aryl, referring to the same residues as aryl, but having two points of attachment.

As used herein, “cycloalkyl” refers to a non-aromatic carbocyclic ring, usually having from 3 to 7 ring carbon atoms. The ring may be saturated or have one or more carbon-carbon double bonds. Examples of cycloalkyl groups include cyclopropyl, cyclobutyl, cyclopentyl, cyclopentenyl, cyclohexyl, and cyclohexenyl, as well as bridged and caged ring groups such as norbornane.

As used herein, “halo” or “halogen” refers to fluoro, chloro, bromo, and iodo, and the term “halogen” includes fluorine, chlorine, bromine, and iodine.

As used herein, “haloalkyl” refers to alkyl as defined above having the specified number of carbon atoms, substituted with 1 or more halogen atoms, up to the maximum allowable number of halogen atoms. Examples of haloalkyl include, but are not limited to, trifluoromethyl, difluoromethyl, 2-fluoroethyl, and penta-fluoroethyl.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl [1,3] dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo [d]thiazolyl, benzothiadiazolyl, benzo [b] [1,4] dioxepinyl, benzo [b] [1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno [3,2-d]pyrimidinyl, benzotriazolyl, benzo [4,6] imidazo [1,2-a]pyridinyl, carbazolyl, cinnolinyl, cyclopenta [d] pyrimidinyl, 6,7-dihydro-5H-cyclopenta [4,5] thieno [2,3-d] pyrimidinyl, 5,6-dihydrobenzo [h] quinazolinyl, 5,6-dihydrobenzo [h] cinnolinyl, 6,7-dihydro-5H-benzo [6,7] cyclohepta [1,2-c]pyridazinyl, dibenzofuranyl, dibenzothiophenyl, furanyl, furanonyl, furo [3,2-c]pyridinyl, 5,6,7,8,9,10-hexahydrocycloocta [d] pyrimidinyl, 5,6,7,8,9,10-hexahydrocycloocta [d] pyridazinyl, 5,6,7,8,9,10-hexahydrocycloocta [d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo [h] quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo [3,4-d]pyrimidinyl, pyridinyl, pyrido [3,2-d]pyrimidinyl, pyrido [3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo [4,5] thieno [2,3-d]pyrimidinyl, 6,7,8,9-tetrahydro-5H-cyclohepta [4,5] thieno [2,3-d]pyrimidinyl, 5,6,7,8-tetrahydropyrido [4,5-c]pyridazinyl, thiazolyl, thiadiazolyl, triazolyl, tetrazolyl, triazinyl, thieno [2,3-d]pyrimidinyl, thieno [3,2-d]pyrimidinyl, thieno [2,3-c] pridinyl, and thiophenyl (i.e. thienyl).

As used herein, the term “a solid support” comprises the solid phase carrier for the synthesis of oligonucleotide, such as CPG.

As used herein, the term “oligonucleotide”, “oligonucleotides” refer to a compound comprising a plurality of linked nucleosides. In certain embodiments, “oligonucleotides” are short, single- or double-stranded DNA or RNA molecules, and include antisense oligonucleotides (ASO), RNA interference (RNAi), and aptamer RNAs. In certain embodiments, one or more of the plurality of nucleosides is modified. In certain embodiment, an oligonucleotide comprises one or more ribonucleosides (as in RNA) and/or deoxyribonucleosides (as in DNA). In some embodiments, the oligonucleotide is a single-stranded oligonucleotide. In some other embodiments, the oligonucleotide is a double-stranded interfering RNA, such as siRNA, aiRNA, shRNA. In some embodiments, the oligonucleotide is circRNA. In some embodiments, the oligonucleotide is mRNA.

As used herein, term “aiRNA” is an asymmetric interfering RNA duplex molecule, comprising an antisense strand and a sense strand, wherein the antisense strand is longer than the sense strand, consists of 19-27 nucleotides, and includes a 3′ overhang of at least one nucleotide, and a 5′ end of 0-8 nucleotides; wherein the antisense strand is at least 70% complementary to a target mRNA; wherein the sense strand consists of 10-26 nucleotides, forms a double-stranded region with the antisense strand where the double-stranded region includes 0, 1 or 2 mismatch pair(s). Exemplary structure of aiRNA is described in US 2009/0208564, which is hereby incorporated by reference in entirety.

As used herein, the term “middle type” refers to an interfering RNA duplex molecule, comprising an antisense strand and a sense strand, where the antisense strand is longer than the sense strand, and comprises both 3′ overhang and 5′ overhang of at least one nucleotide.

As used herein, the term “blunt type” refers to an interfering RNA duplex molecule, comprising an antisense strand and a sense strand, where the RNA duplex molecule has at least one blunt end, preferably having one blunt end at the 3′ end of the sense strand or at the 5′ end of antisense strand.

As used herein, the term “modified oligonucleotide” means an oligonucleotide comprising at least one modified nucleotide.

As used herein, the term “modified nucleotide” means a nucleotide having at least one modified sugar moiety, modified internucleoside linkage, and/or modified nucleobase.

As used herein, the term “modified nucleoside” means a nucleoside having at least one modified sugar moiety, and/or modified nucleobase.

As used herein, the term “naturally occurring internucleoside linkage” means a 3′ to 5′ phosphodiester linkage.

As used herein, the term “modified internucleoside linkage” refers to a substitution or any change from a naturally occurring internucleotide bond. For example, a phosphorothioate linkage is a modified internucleotide linkage.

As used herein, the term “natural sugar moiety” means a sugar found in DNA (2-H) or RNA (2-OH).

As used herein, the term “modified sugar” refers to a substitution or change from a natural sugar. For example, a 2′-O-methoxyethyl modified sugar is a modified sugar.

As used herein, the term “bicyclic sugar” means a furosyl ring modified by the bridging of two non-geminal ring atoms. A bicyclic sugar is a modified sugar.

As used herein, the term “modified nucleobase” refers to any nucleobase other than adenine, cytosine, guanine, thymidine, or uracil. For example, 5-methylcytosine is a modified nucleobase. An “unmodified nucleobase” means the purine bases adenine (A) and guanine (G), and the pyrimidine bases thymine (T), cytosine (C), and uracil (U).

As used herein, “prevention” and “preventing” are used interchangeably. These terms refer to an approach for obtaining beneficial or desired results including but not limited to a prophylactic benefit. For “prophylactic benefit”, the conjugates or compositions may be administered to a patient at risk of developing a particular disease, or to a patient reporting one or more of the physiological symptoms of a disease, even though a diagnosis of this disease may not have been made.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably. As used herein, a “pharmaceutical composition” includes a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.nce to a human subject.

### Compounds Configuration

Compounds of the present invention, and salts thereof, may exist in their tautomeric form (for example, as an amide or imino ether). All such tautomeric forms are contemplated herein as part of the present invention.

All stereoisomers of the compounds of the present invention (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 95% (e.g., “substantially pure” compound I), which is then used or formulated as described herein. In certain embodiments, the compounds of the present invention are more than 99% pure.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

### D-Amino Acids/L-Amino Acids

Amino acids contained within the peptides or polypeptides described herein will be understood to be in the L- or D-configuration.

### II. Embodiments

The present invention provides novel compounds with novel linker compositions for linking various components of the compound. The compound conjugates an oligonucleotide with one or more targeting ligands. The oligonucleotide can be naturally occurring (isolated from nature, or synthesized in a laboratory) or chemically modified in at least one subunit.

In some embodiments, the oligonucleotide is chemically modified oligonucleotide. In some embodiments, chemically modified oligonucleotide comprises backbone modification (or internucleoside linkage modification, such as phosphate group modification), ribose group modification, base modification.

In certain embodiments, the oligonucleotide has at least one phosphorothioate internucleoside linkage, or at least one methylphosphonate internucleoside linkage, or at least one other modified internucleoside linkage such as:

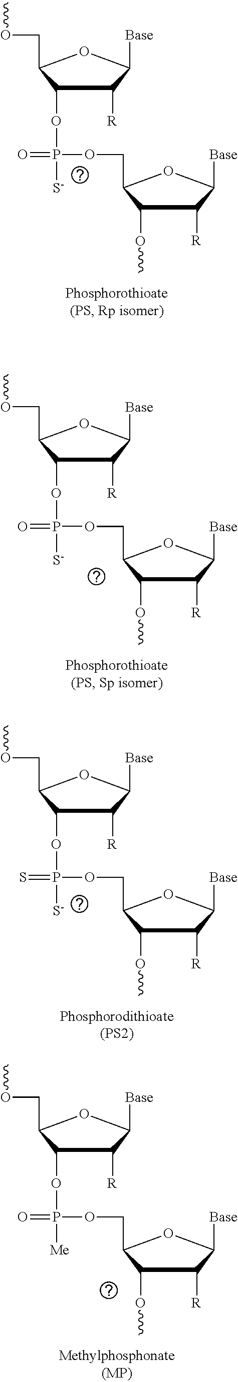

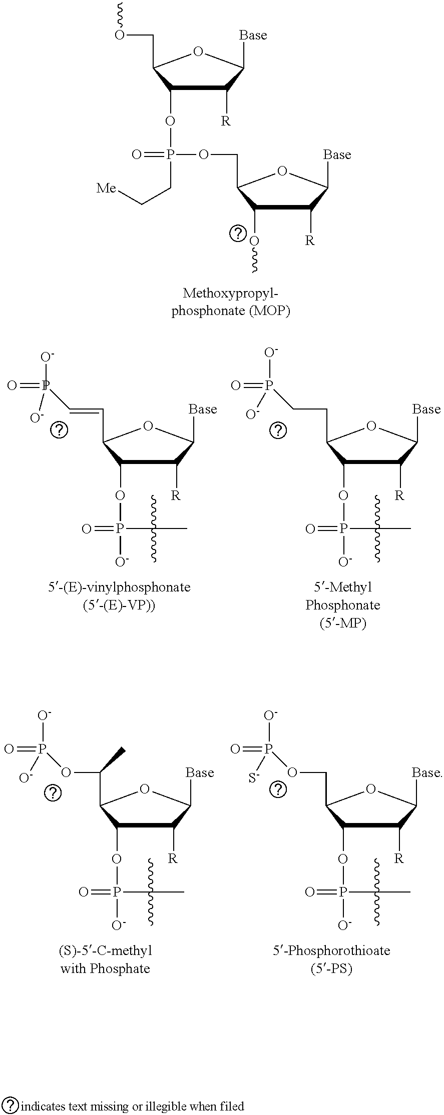

In certain embodiments, the oligonucleotide has at least one chemically modified nucleotide with ribose modification. In certain embodiments, the 2′ position of the modified ribose moiety is replaced by a group selected from OR, R, halo, SH, SR, NH₂, NHR, NR₂, or CN, where each R is independently C₁-C₆ alkyl, alkenyl or alkynyl, and halo is F, Cl, Br or I. In some embodiments, the 2′ position of the modified ribose moiety is replaced by a group selected from allyl, amino, azido, thio, O-allyl, O—C₁-C₁₀ alkyl, OCF₃, OCH₂F, O(CH2)₂SCH₃, O(CH₂)₂—O—N(Rₘ)(Rₙ), O—CH₂—C(═O)—N(Rₘ)(Rₙ), or O—CH₂—C(═O)—N(R₁)—(CH₂)₂—N(Rₘ)(Rₙ), where each R₁, Rₘ and Rₙ is, independently, H or substituted or unsubstituted C₁-C₁₀ alkyl. In some embodiments, the modified ribose moiety is selected from the group of 5′-vinyl, 5′-methyl (R or S), 4′-S, 2′-F, 2′-OCH₃, 2′-OCH₂CH₃, 2′-OCH₂CH₂F and 2′-O(CH₂)₂CH₃ substituent groups. In some embodiments, the modified ribose moiety is substituted by bicyclic sugar selected from the group of 4′-(CH₂)—O-2′ (LNA); 4′-(CH₂)—S-2; 4′-(CH₂)₂—O-2′ (ENA); 4′-CH(CH₃)—O-2′ (cEt) and 4′-CH(CH₂OCH₃)—O-2′,4′-C(CH₃)(CH₃)—O-2′,4′-CH₂—N(OCH₃)-2′,4′-CH₂—O N(CH₃)-2′,4′-CH₂—N(R)—O-2′, where R is H, C1-C12 alkyl, or a protecting group, 4′-CH₂—C(H)(CH₃)-2′, and 4′-CH₂—C—(═CH₂)-2′. In some embodiments, the modified sugar moiety is selected from the group of 2′-O-methoxyethyl modified sugar (MOE), a 4′-(CH₂)—O-2′ bicyclic sugar (LNA), 2′-deoxy-2′-fluoroarabinose (FANA), and a methyl(methyleneoxy) (4′-CH(CH₃)—O-2) bicyclic sugar (cEt). In certain embodiments, the oligonucleotide has a chemically modified nucleotide selected from the group consisting, 2′-methoxyethyl, 2′-OCH₃ and 2′-fluoro.

The oligonucleotide can be conjugated to the rest of the compound, or the “backbone” at the 5′ and/or 3′ end of the oligonucleotide. The conjugated oligonucleotide can be delivered as a single strand or hybridized to a substantially complementary oligonucleotide as part of a duplex. The substantially complementary oligonucleotide can be similarly conjugated or not.

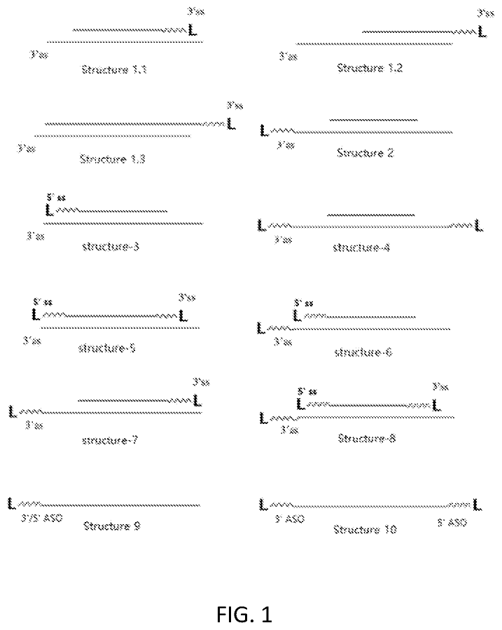

In an embodiment, the conjugated oligonucleotide forms part of a siRNA duplex (either the sense or antisense strand, or both). In a preferred embodiment, the conjugated oligonucleotide forms part of an aiRNA duplex (either the sense or antisense strand, or both). In another embodiment, the oligonucleotide that is conjugated according to principles of the invention is used as an antisense oligonucleotide (ASO). In yet another embodiment, the oligonucleotide that is conjugated according to principles of the invention is used as a micro-RNA (miRNA) molecule. Some exemplary embodiments of the conjugated oligonucleotide as shown in FIG. 1. For duplex RNA molecule, preferably, the oligonucleotide can be conjugated to the rest of the compound, or the “backbone” at the 3′ end of the sense strand.

### Embodiment 1

In a first feature, an oligonucleotide is conjugated to a backbone containing multiple components including a terminus where a cluster of more than one ligand (e.g., GalNAc), e.g., 2-8 and preferably 3, are attached to the backbone, directly or through one or more intermediate linkers, at an attachment point provide by a residue derived from a histidine residue.

In one embodiment, the compound of the present invention has the structural formula as shown in (G-H1), (G-H1-01)-(G-H1-11).

In one embodiment, the compound of the present invention has the structure as shown in HC-1 to HC-9.

In one embodiment, optionally, the configuration of the compound is R isomer or its mixture. In one embodiment, the configuration of the compound means the isomer of the chiral carbon atom shown in the structural formula.

In the compound of the present invention, the naturally occurring or chemically modified oligonucleotide is linked to the rest of the compound through its 5′ end and/or its 3′ end.

### Embodiment 2

In a second feature, an oligonucleotide is conjugated to a backbone containing multiple components including a terminus where a cluster of more than one ligand (e.g., GalNAc), e.g., 2-8 and preferably 3, are attached to the backbone, directly or through one or more intermediate linkers, at an attachment point provide by a moiety derived from a glutamic acid residue.

In one embodiment, the compound of the present invention has the structural formula as shown in (G-G1), (G-G1-01)-(G-G1-10).

In one embodiment, the compound of the present invention has the structure as shown in GC-1 to GC-9.

In one embodiment, optionally, the configuration of the compound is R isomer or racemate. In one embodiment, the configuration of the compound means the isomer of the chiral carbon atom shown in the structural formula.

### III. Examples

### Synthesis

In some embodiments, the compound shown in formula G-H1, G-H1-01˜G-H1-11, G-G1, G-G1-01˜G-H1-10, with an oligonucleotide conjugated to a backbone containing multiple components including a terminus where a cluster of more than one ligand (e.g., GalNAc), e.g., 2-8, is synthesized by a cluster backbone comprising three or more reactive moieties reacting with ligands and oligonucleotides.

### Example 1 Synthesis of GC-05 (Glu(R)-Cluster GalNAc)

Synthesis of GC-05 (Glu(R)-Cluster GalNAc) was shown as below 5 Steps:

### Step 1: The Synthesis Route of Intermediate N-2

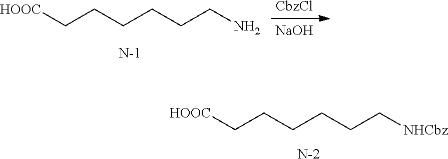

30 g of compound N-1 was dissolved in 420 mL of 2 M NaOH, 30 mL of THF was added. Cooled to 0-5° C. in an ice bath, and 35 g of CbzCl was added by dripping slowly. After the dropping was completed, stirred at room temperature for 1 h, the reaction completed was monitored by UPLC-MS (Ultra Performance Liquid Chromatography-Mass Spectrum). Reaction solution was washed with MTBE (200 mL*3), the water phase was separated and EA was added, adjusted pH to 4 with 3 M HCl after cooling, the EA phase extracted and separated, the water phase extracted with 300 mL EA once again, combined the organic phases, washed with saturated sodium chloride solution for 3 times, dried over anhydrous sulfuric acid sodium, and concentrated to obtain 51.58 g of compound N-2. MS (ESI) m/z 278.05 ([M−H]⁻).

### Step 2: The Synthesis Route of Intermediate M-3

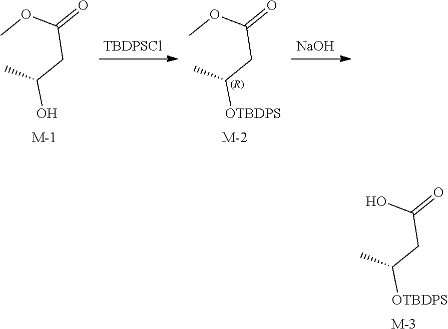

### Synthesis of Compound M-2

Under nitrogen atmosphere, 20 g M-1 ((R)-3-hydroxy-methyl butyrate) was dissolved in 200 mL DCM, 18 g imidazole was added, and 56.2 g TBDPSCl was dropped. The reaction was performed for 2 h at room temperature after dropping completed. TLC was used to monitor the reaction complete. 200 mL saturated ammonium chloride was added to quench, DCM layer was separated, washed once with saturated sodium chloride, dried over anhydrous sodium sulfate, concentrated and dried, obtained 73 g of M-2 crude product. MS (ESI) m/z 357.10 ([M+H]⁺)

### Synthesis of Compound M-3

13.6 g sodium hydroxide was dissolved in 80 mL water to preparation aqueous solution of sodium hydroxide, and 73 g M-2 crude product was dissolved in 500 mL methanol, then the aqueous solution of sodium hydroxide was added. After stirring at 35° C. for 15 h, TLC was used to monitor the reaction complete. Methanol was removed by concentration at 40° C., 500 mL ethyl acetate was added, pH was adjusted by 2 M HCl, EA layer was separated, washed with saturated sodium chloride, dried over anhydrous sodium sulfate, concentrated, purified by silica gel column chromatography (PE:EA=50:1-10:1), and 47.8 g of M-3 was obtained from the concentrated product. MS (ESI) m/z 341.10 ([M−H]⁻). ¹H-NMR (400 MHz, CDCl₃) δ 7.66-7.69 (m, 4H), 7.35-7.45 (m, 6H), 4.23-4.30 (m, 1H), 2.43-2.56 (m, 2H), 1.14 (d, 3H), 8.77 (s, 9H).

### Step 3: The Synthesis Route of Intermediate S-04

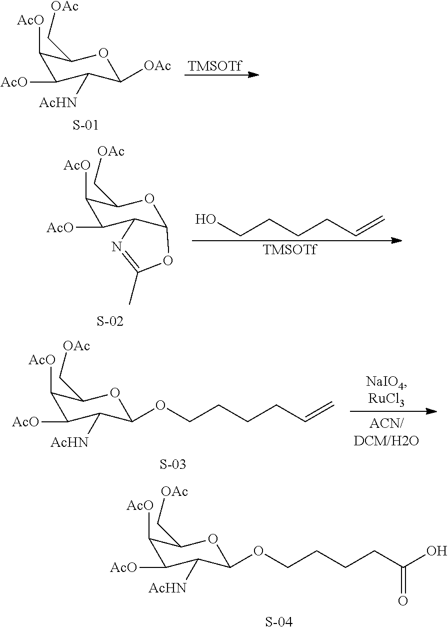

### Synthesis of Compound S-02

Under nitrogen atmosphere, 250 g of compound S-01 was dissolved in 2 L of anhydrous DCM, and 158 g of TMSOTf was added introduced by dripping under ice bath. After the dropping was completed, the temperature was raised to 40° C. and stirred for 2 hours, the reaction was completed by TLC monitoring. Cooled in an ice bath, 1.7 L of saturated sodium bicarbonate aqueous solution was added by dropping, the organic phase was separated and washed with saturated sodium chloride solution, dried over anhydrous sodium sulfate, filtered, and the filtrate was concentrated to obtain 211 g of yellow oily product S-02, ESI-MS m/z: [M+H]⁺=430.12

### Synthesis of Compound S-03

Under nitrogen atmosphere, 169 g of compound S-02 was dissolved in 1.2 L of anhydrous DCM, 56.6 g of 5-hexen-1-alcohol was added, 57.38 g of TMSOTf was added by dripping in an ice-water bath, the reaction was performed overnight at room temperature after the dropping was completed. The reaction completed was monitored by TLC, cooled in an ice-water bath, saturated NaHCO₃ was added by dropwise to adjust pH to 7-8, and then the organic phase was extracted and separated. The organic phase was washed once with saturated NaCl, dried over anhydrous sodium sulfate, filtered, and the filtrate was concentrated to dryness and purified by column chromatography, gradient eluted with DCM:MeOH=80:1-40:1, the product eluent was collected and concentrated to dryness to obtain 94 g of oily product S-03, ESI-MS m/z: [M+H]⁺=430.23.

### Synthesis of Compound S-04

82 g of compound S-03 was weighed, 410 mL of acetonitrile, 410 mL of DCM and 573 mL of H₂O was added, stirred for dissolution, cooled to 5-10° C. in an ice bath, and 163 g of NaIO₄ and 2.37 g of RuCl₃ were added to the reaction system. After stirring at room temperature for 2 hours, the reaction completed was monitored by TLC. The reaction solution was filtered through celite, and the filtrates were combined, and the aqueous phase was extracted and separated. The organic phase was washed three times with saturated NaHSO₃ (200 mL×3). The aqueous phases were combined, 600 mL of DCM was added, and the pH was adjusted to 2-3 with 2 M HCl, the DCM phase was extracted and separated, dried over anhydrous sodium sulfate, filtered, and concentrated to obtain 76 g of gray solid compound S-04, ESI-MS m/z: [M−H]⁻=446.17, 1H-NMR (400 MHz, DMSO-d6): δ 11.97 (s, 1H, COOH); 7.79 (d, 1H, NH); 5.20 (d, 1H), 4.95 (dd, 1H); 4.48 (d, 1H); 4.05-3.98 (m, 3H); 3.86 (dt, 1H); 3.74-3.65 (m, 1H, —OCH₂—CH₂); 3.45-3.37 (m, 1H, —OCH₂—CH₂); 2.19 (t, 2H, —CH₂—COOH); 2.09 (s, 3H, —COCH₃); 1.99 (s, 3H, —COCH₃); 1.88 (s, 3H, —COCH₃); 1.76 (s, 3H, —COCH₃); 1.55-1.45 (m, 4H, 2x(—CH₂)).

### Step 4: The Synthesis Route of Intermediate G-6

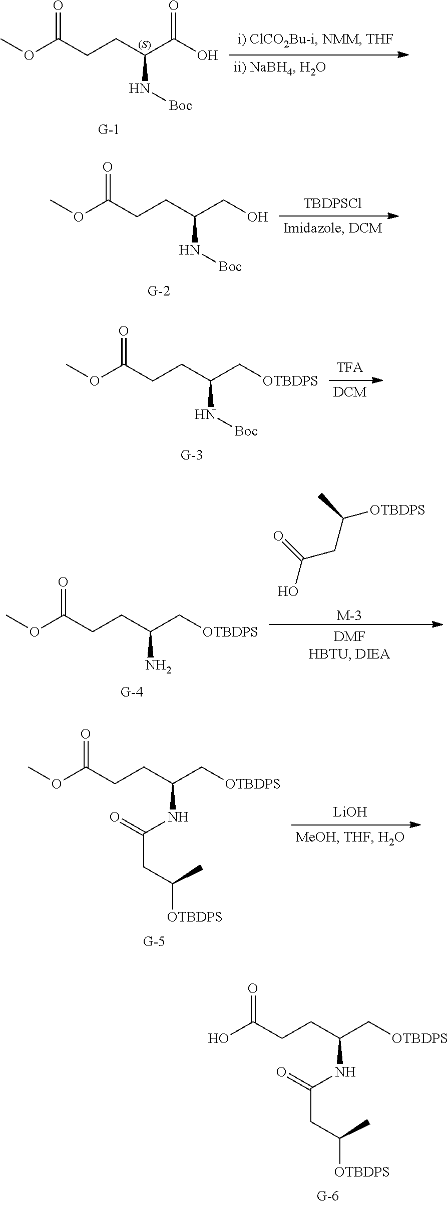

### Synthesis of Compound G-2

50 g of compound G-1 ((S)—N-Boc-glutamic acid methyl ester) was dissolved in 500 mL of THF, 25.2 mL of NMM was introduced by dripping in an ice-water bath, and 26.6 mL of isobutyl chloroformate was introduced by dripping after stirring for 5 minutes. Stirred for 1 hour sequentially after the dropping was completed. Suction filtration was taken, the filtrate was collected, and 8.73 g of NaBH₄ was added to the filtrate under an ice-water bath. After the addition was completed, the reaction was continued for 2 hours, sample was taken to monitor the reaction completed by TLC, 250 mL of water was added and 500 mL of EA were added for extraction. The organic phase was washed with saturated sodium chloride solution, dried over anhydrous sodium sulfate, and concentrated to obtain 45.35 g of oily product G-2. MS (ESI) m/z 248.19 ([M+H]⁺).

### Synthesis of Compound G-3

45.35 g of compound G-2 was dissolved in 500 mL of DCM, 31.2 g of imidazole was added, and 91.1 g of TBDPSCl was introduced by dripping. After the dropping was completed, the mixture was stirred at room temperature for 2 h, and the reaction completed was monitored by TLC. 150 mL of water was added to the reaction liquid, and the DCM layer was extracted and separated, washed once with saturated sodium chloride solution, dried over anhydrous sodium sulfate, and concentrated to obtain an oily product. Purified by silica gel column chromatography, gradient eluted with PE:EA=40:1-10:1, and concentrated to obtain 36.7 g of colorless transparent oily product G-3. MS (ESI) m/z 486.66 ([M+H]⁺).

### Synthesis of Compound G-4

46.15 g of compound G-3 was dissolved in 500 mL of DCM, and 70 mL of TFA (trifluoroacetic Acid) was introduced by dripping in an ice-water bath. After the dropping was completed, reaction was stirred at room temperature for reaction 4 hours. The reaction completed was monitored by TLC, concentrated under reduced pressure, 500 mL of DCM was introduced by dripping to the remains, saturated sodium bicarbonate solution was added by dripping to adjust pH to 8, the DCM layer was extracted and separated, washed once with saturated sodium chloride solution, dried over anhydrous sodium sulfate, and concentrated to dryness to obtain 36.7 g of light yellow oil G-4. MS (ESI) m/z 386.51 ([M+H]⁺).

### Synthesis of Compound G-5

31.9 g of compound M-3 was dissolved in 300 mL DMF, 42 g HBTU and 23 mL DIEA were added, stirred at room temperature for 10 minutes, then 35 g of compound G-4 was added, moved to room temperature and stirred for 1 hour. TLC was used to monitor the reaction complete. The reaction solution was extracted with 600 mL saturated sodium bicarbonate and 400 mL EA. The EA layer was separated and washed with saturated salt water, dried over anhydrous sodium sulfate, and concentrated under reduced pressure to obtain an oil. Crude residue was purified by column chromatography (PE:EA=20:1-8:1) to produce white solid product G-5 (41 g). MS (ESI), m/z 710.33 ([M+H]⁺).

### Synthesis of Compound G-6

33.3 g of compound G-5 was added to a mixture of 100 mL MeOH, 100 mL THE and 100 mL water, and 5.92 g lithium hydroxide monohydrate was added, and reacted at room temperature for 8 hours. Reaction was monitor by TLC, the reaction mixture was concentrated under reduced pressure, then 400 mL ethyl acetate was added to the concentrated remains, adjusted the pH to 4-5 using dilute hydrochloric acid solution, extracted and separated organic phase, washed with saturated sodium chloride solution once, dried over anhydrous sodium sulfate, concentrated under reduced pressure, 33.8 g of colorless transparent oil product G-6 was obtained. MS (ESI) m/z: 694.35 ([M+H]⁺).

### Step 5: The Synthesis Route of Compound GC-05

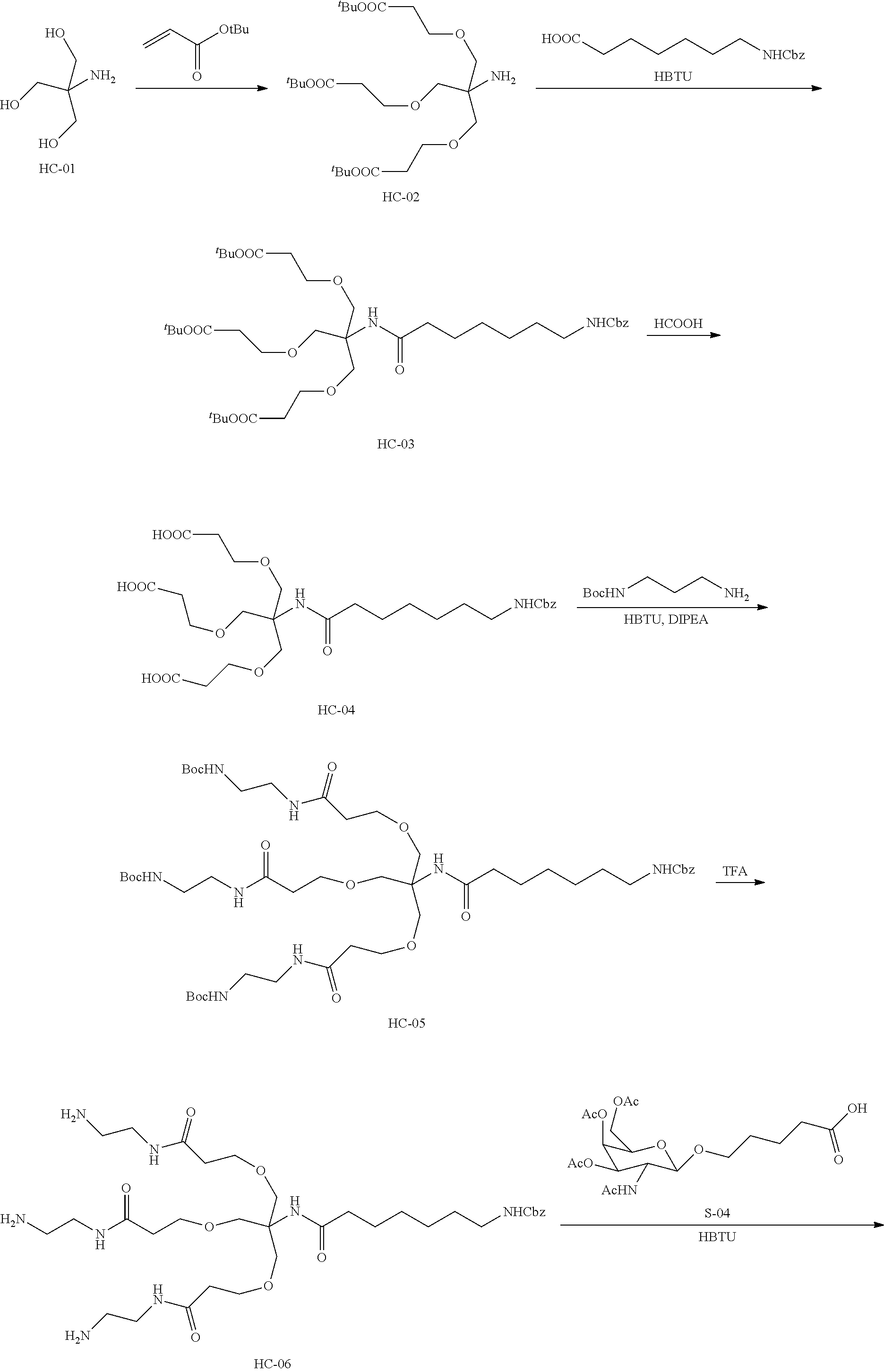

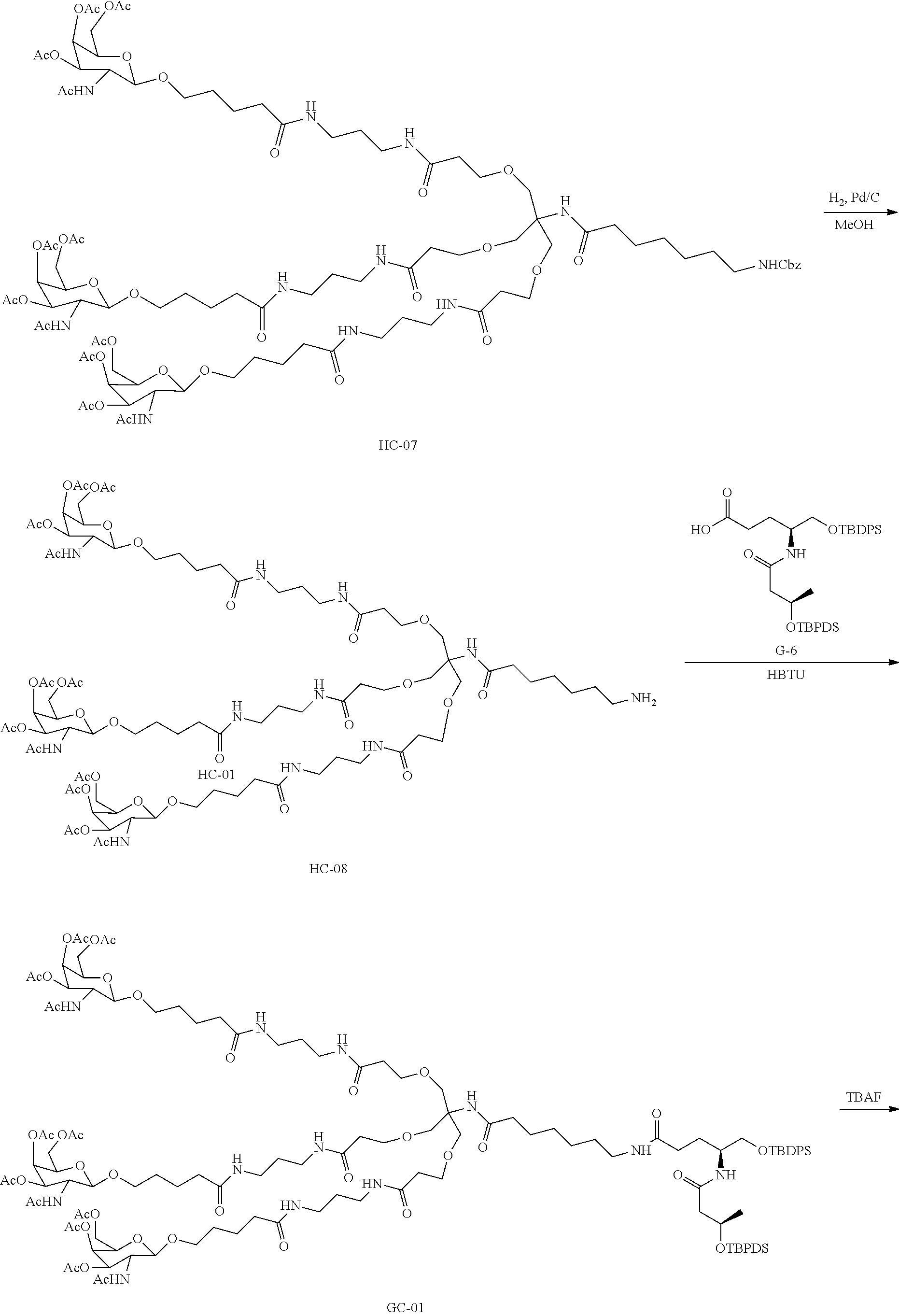

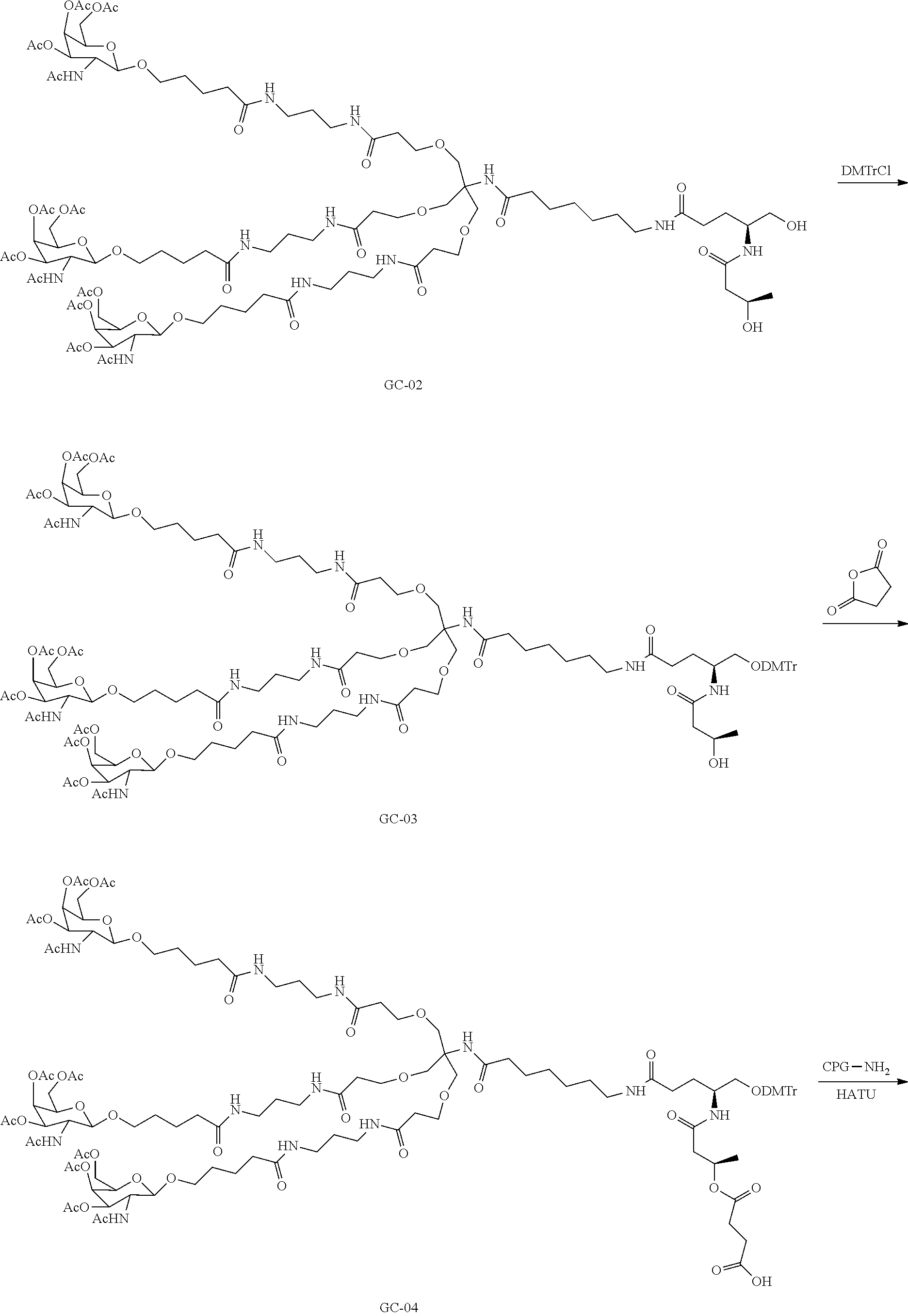

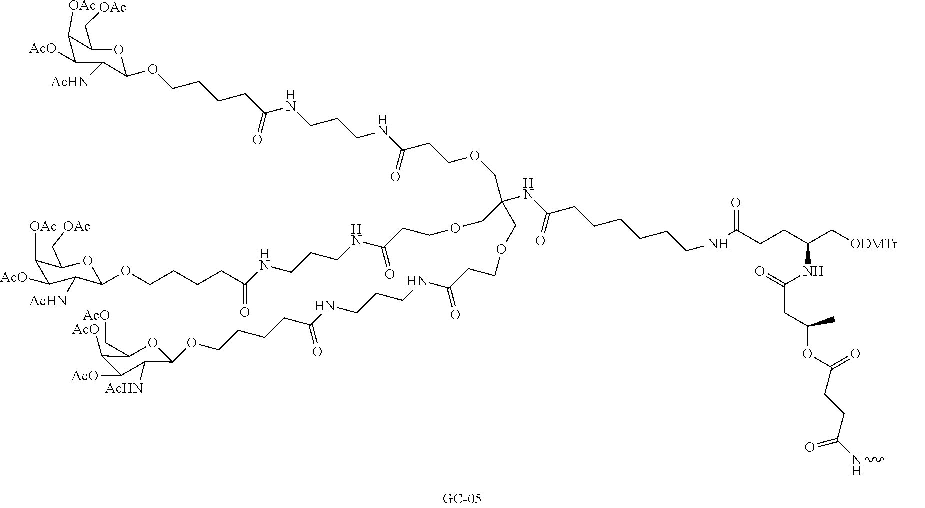

### Synthesis of Compound HC-02

100 g of compound HC-01 (Trimethylol aminomethane) was dissolved in 250 mL of DMSO, and then 16.5 mL of 5 M NaOH was added. In the ice-water bath, 400 g of tert-butyl acrylate was introduced by dropping slowly to the mixture. After the dropping was completed, the reaction was completed after stirring at room temperature for 18 hours. 2 L of saturated sodium chloride solution and 500 mL of ethyl acetate were added to the reaction liquid, and the organic phase was separated after stirring. The organic phase was washed twice with saturated sodium chloride solution, dried with anhydrous sodium sulfate, filtered and concentrated to dryness to obtain crude compound HC-02. It was purified by silica gel column chromatography, and gradient eluted with petroleum ether:ethyl acetate=10:1-1:1. The product eluate was collected and concentrated to dryness to obtain 260 g of compound HC-02. MS (ESI) m/z 506.35 ([M+H]⁺).

### Synthesis of Compound HC-03

23.24 g of intermediate N-2 was dissolved in 200 mL of DMF, and then 16 g of DIPEA and 38 g of HBTU were added, and after stirring at room temperature for 20 minutes, 50.47 g of compound HC-02 dissolved in 50 mL of DMF was introduced by dropping slowly. After the dropping was completed, the mixture was stirred at room temperature for 1 hour, and the reaction completed was monitored by TLC. 600 mL of saturated sodium bicarbonate was added to the reaction liquid, extracted with 300 mL of ethyl acetate, the organic phase was separates, washed with saturated sodium chloride solution, dried over anhydrous sodium sulfate, concentrated, purified by silica gel column chromatography, and gradient eluted with petroleum ether:ethyl acetate=8:1-4:1. The product eluent was collected and concentrated to dryness to obtain 43 g of light yellow oil. MS (ESI) m/z 767.51 ([M+H]⁺).

### Synthesis of Compound HC-04

1280 mL of formic acid was added to 160 g of compound HC-03, stirred at room temperature for 6 hours, the reaction completed was monitored by TLC, and the formic acid was evaporated under reduced pressure to obtain the light yellow oil. 300 mL of toluene was added to the remains, and toluene was evaporated under reduced pressure to obtain 111.85 g of oily compound HC-04. ¹HNMR (400 MHz, DMSO-d6) δ12.18 (brs, 3H), 7.37-7.30 (m, 5H), 7.20 (t, 1H), 7.92 (s, 1H), 5.00 (s, 2H), 3.58-3.55 (m, 12H), 2.97 (q, 2H), 2.37-2.47 (m, 6H), 2.04 (t, 2H), 1.46-1.36 (m, 4H), 1.17-1.29 (m, 4H). MS (ESI) m/z 597.29 ([M−H]⁻)

### Synthesis of Compound HC-05

110.72 g of compound HC-04 was dissolved in 800 mL of DMF, and 107.48 g of DIPEA and 253.28 g of HBTU were added. After the mixture was stirred at room temperature for 20 minutes, 101.3 g of tert-Butyl (3-aminopropyl-carbamate dissolved in 200 mL of DMF was introduced by dropping slowly. After the dropping was completed, the mixture was stirred at room temperature for 1 hour, and the reaction completed was monitored by TLC. 2 L of saturated sodium bicarbonate aqueous solution was added to the reaction liquid, extracted with 800 mL of ethyl acetate, the organic phase was washed with saturated sodium chloride solution, dried over anhydrous sodium sulfate, concentrated under reduced pressure, purified by silica gel column chromatography, and gradient eluted with dichloromethane: methanol=80:1-20:1 The product eluent was collected, concentrated to dryness to obtain 220 g of oily compound HC-05. MS (ESI) m/z 1067.70 ([M+H]⁺).

### Synthesis of Compound HC-06

79.68 g of compound HC-05 was dissolved in 880 mL of DCM, cooled in an ice-water bath, 190 g of trifluoroacetic acid was introduced by dripping. After the dropping was completed, the mixture was stirred at room temperature for 4 hours, and the reaction completed was monitored by UPLC-MS. DCM was evaporated under reduced pressure, 600 mL of toluene was added, concentrated to dryness to obtain 121.6 g of oily compound HC-06. MS (ESI) m/z 767.61 ([M+H]⁺).

### Synthesis of Compound HC-07

90 g of compound S-04 was dissolved in 265 mL of DMF, 72 g of DIPEA was added, 52 g of HBTU was added, 62 g of compound HC-06 dissolved in 250 mL of DMF and 72 g of DIPEA was introduced by dripping slowly after stirring for 20 minutes. 1000 mL of saturated sodium bicarbonate was added to the reaction liquid, 500 mL of ethyl acetate was added, the ethyl acetate layer was extracted and separated, washed with saturated sodium chloride solution, dried with anhydrous sodium sulfate, concentrated, purified by silica gel column chromatography, gradient eluted with dichloromethane: methanol=50:1-10:1. The product eluent was collected, concentrated to dryness to obtain 75 g of white solid compound HC-07. MS (ESI) m/z 1028.46 ([(M+2)/2]⁺).

### Synthesis of Compound HC-08

10 g of compound HC-07 was dissolved in 80 mL of methanol, 1 g of 10% Pd (Palladium)/C was added, replaced the air with hydrogen for three times and stirring at room temperature for 8 h. Pd/C was filtered off. The filtrate was concentrated and dried to obtain 8.5 g of an off-white solid compound HC-08. MS (ESI) m/z 961.37 ([(M+2)/2]⁺).

### Synthesis of Compound GC-01

2.0 g of intermediate G-6 was dissolved in 30 mL of DMF, 1.41 g of HBTU and 557 mg of DIEA were added, stirred at room temperature for 20 minutes under nitrogen atmosphere, and 6.62 g of compound HC-08 was added. After stirring at room temperature for 1 hour, sample was taken to monitor the reaction completed by TLC. 200 mL of saturated sodium bicarbonate and 100 mL of ethyl acetate were added to the reaction liquid, and the organic phase was extracted and separated. The organic phase was washed once with saturated sodium chloride solution, dried over anhydrous sodium sulfate, concentrated and purified by column chromatography, and gradient eluted with DCM: MeOH=30:1-10:1 The product eluent was collected and concentrated to dryness to obtain 4.93 g of white solid compound GC-1. MS (ESI) m/z 866.72 ([(M+3)/3]⁺).

### Synthesis of Compound GC-02

20.4 g of compound GC-01 was dissolved in 200 mL of THF, 25 mL of 1 M TBAF-THF (tetrabutylammonium fluoride) solution was added, and the mixture was stirred at room temperature overnight. The reaction liquid was concentrated and purified by column chromatography, gradient eluted with DCM: MeOH=50:1-10:1, the product eluent was concentrated to obtain 10.39 g of white solid compound GC-02. MS (ESI) m/z 1061.55 ([(M+2)/2]⁺).

### Synthesis of Compound GC-03

Under nitrogen atmosphere, 5.14 g of compound GC-02 was dissolved in 40 mL of anhydrous pyridine, and 4.43 g of DMTrCl was added. After reaction at room temperature for 0.5 h, sample was taken to monitor the reaction completed, and 10 mL of methanol was added to quench the reaction. The reaction liquid was concentrated, purified by column chromatography, gradient eluted with DCM: MeOH=50:1-10:1, 4.54 g of white solid compound GC-3 was obtained. MS (ESI) m/z 1061.59 ([(M-302)+2)/2]⁺).

### Synthesis of Compound GC-04

Under nitrogen atmosphere, 3.5 g of compound GC-03 was dissolved in 30 mL of anhydrous pyridine, 10 mg of DMAP and 1.57 g of succinic anhydride were added, and the mixture was stirred at room temperature for 72 hours, the reaction completed was monitored by TLC. The pyridine was concentrated to obtain the crude product, purified by silica gel column chromatography, gradient eluted with DCM: MeOH=50:1-10:1, the product eluent was concentrated to obtain 3.61 g of white solid compound GC-04. MS (ESI) m/z 1111.50 ([(M-302)+2)/2]⁺). ¹HNMR (400 MHz, DMSO-d6) δ 7.89-7.82 (m, 6H), 7.76-7.74 (m, 4H), 7.38-7.36 (m, 2H), 7.31-7.27 (m, 2H), 7.24-7.16 (m, 5H), 6.99 (s, 1H), 6.88-6.81 (m, 4H), 5.21 (d, 2H), 4.98-4.95 (m, 3H), 4.50-4.47 (m, 3H), 4.04-4.01 (m, 10H), 3.91-3.86 (m, 6H), 3.73-3.69 (m, 12H), 3.54-3.52 (m, 12H), 3.41-3.39 (m, 4H), 3.04-3.02 (m, 12H), 2.68-2.63 (m, 9H), 2.29-2.26 (m, 8H), 2.10 (s, 9H), 2.06-2.02 (m, 8H), 1.99 (s, 9H), 1.89 (s, 9H), 1.77 (s, 9H), 1.52-1.44 (m, 18H), 1.11 (d, 3H).

### Synthesis of Compound GC-05

246 mg of compound GC-04 was dissolved in 10 mL of anhydrous acetonitrile, 46 mg of HATU and 27 mg of DIPEA were added, and was shaken in a shaker for 10 minutes. 800 mg of LCAA-CPG (100 μmol/g) was added to the above-mentioned reaction liquid, and was shaken overnight at room temperature. The second day, the reaction liquid was filtered, and the filter cake was washed with acetonitrile. 5 mL CapA (20% NMI-80% ACN) and 5 mL CapB (20% AC₂O-30% lutidine-50% CAN) were added to the filter cake, and shaken in a shaker sequentially at room temperature for 2 hours. The reaction liquid was filtered, and the filter cake was washed with acetonitrile, and dried under vacuum at room temperature for 2 hours to obtain 780 mg of compound HC-13 with a measured load of 45 mol/g.

### Example 2 Synthesis of HC-13 (his(R)-Cluster GalNAc)

Synthesis of HC-13 (His(R)-Cluster GalNAc) was show below (2 steps):

### Step 1: The Synthesis Route of Compound H-09

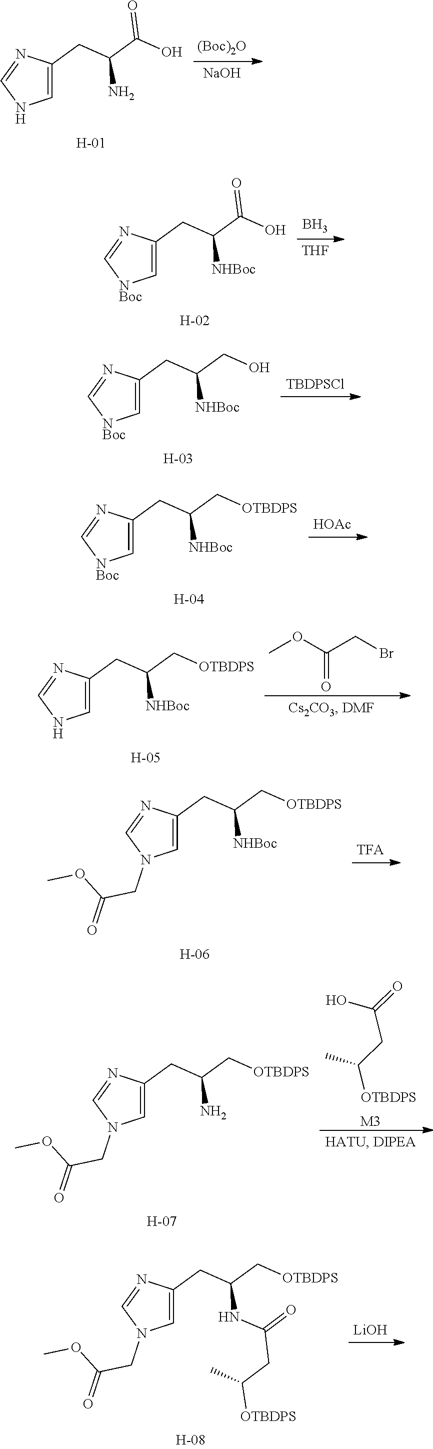

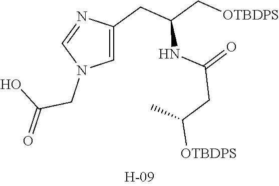

### Synthesis of Compound H-02

38.4 g sodium hydroxide was dissolved in 400 mL water, and 400 mL THF was added. The mixture was cooled in ice water bath, 50 g L-H-01 (Histidine) was added to it and dissolved by stirring. 175 g di-tert-butyl dicarbonate was added and stirred at room temperature for 4 h. After the reaction was complete monitored by TLC, the reaction mixture was filtrated under reduced pressure and concentrated. 200 mL MTBE was added to the concentrated residue for washing and extraction for 3 times. The water layer was separated, 500 mL ethyl acetate was added, the pH was adjusted to 2-4 with 3 M hydrochloric acid. The EA layer was separated, washed with saturated sodium chloride, dried over anhydrous sodium sulfate, filtered, and the filtrate was concentrated and dried to obtain 98 g white solid. MS (ESI) m/z 356.25 ([M+H]⁺).

### Synthesis of Compound H-03

110 g compound H-02 was dissolved in 880 mL anhydrous THE under nitrogen atmosphere. Cooled to 0-5° C. in ice bath, and 1.1 L borane tetrahydrofuran solution (1 M) was dropped slowly to reaction solution, stirred at room temperature for 1 h. The reaction was completely monitored by TLC. After temperature of the reaction mixture was cooled to 0-10° C. in ice bath, the reaction was quenched by 230 mL methanol with added dropwise. THF was concentrated, 800 mL 1 ethyl acetate and 300 mL saturated sodium chloride solution were added to the residue to extract, and the organic phase dried over anhydrous sodium sulfate, filtered, and the filtrate was concentrated and dried to obtain 106 g compound H-03 crude product. MS (ESI) m/z 342.40 ([M+H]⁺), ¹H-NMR (400 MHz, DMSO-d6) δ 8.69 (s, 1H), 7.37 (s, 1H), 6.68 (d, 1H), 4.82 (s, 1H), 3.68-3.82 (m, 1H), 3.29-3.43 (m, 2H), 2.89 (dd, 1H), 2.54 (d, 1H), 1.57 (s, 9H), 1.34 (s, 9H).

### Synthesis of Compound H-04

106 g compound H-03 was dissolved in 1 L dichloromethane, 38 g imidazole was added, 128 g TBDPSCl was added dropwise into the reaction solution. After dropping, the reaction was reacted at room temperature for 1 h. TLC was used to monitor the reaction complete. 400 mL saturated sodium chloride was added to the reaction solution, and DCM layer was extracted, washed with saturated sodium chloride, dried over sodium sulfate, concentrated, purified by silica gel column chromatography with gradient elution, (petroleum ether:ethyl acetate=100:1-20:1). The product eluent was collected, condensed and dried to obtain 106 g white solid. ¹H-NMR (400 MHz, DMSO-d6) δ 8.71 (s, 1H), 7.63-7.65 (m, 4H), 7.41-7.48 (m, 6H), 6.85 (d, 1H), 3.91-4.02 (m, 1H), 3.61 (d, 2H), 3.04 (dd, 1H), 2.57-2.63 (m, 1H), 1.58 (s, 9H), 1.35 (s, 9H), 1.01 (s, 9H).

### Synthesis of Compound H-05

80 g compound H-04 was dissolved in 240 mL glacial acetic acid and stirred overnight at 80° C. After the reaction was complete monitored by TLC, the mixture was cooled in ice water bath, then 400 mL ethyl acetate was added and 4 M sodium hydroxide was added dropwise to adjust the pH to 8-9. Organic phase was separated, and washed once with saturated sodium chloride, dried over anhydrous sodium sulfate, condense and dried to obtain 66 g white solid. MS (ESI) m/z 480.27 ([M+H]⁺).

### Synthesis of Compound H-06

Under the nitrogen atmosphere, 37.8 g compound H-05 was dissolved in 250 mL DMF, and 33.4 g cesium carbonate was added, and stirred at room temperature for 30 min. 14.5 g methyl bromoacetate dissolved in 40 mL DMF was added dropwise to the solution. After dropping, stirred at room temperature for 0.5 h, UPLC-MS was used to monitor the reaction basically complete. 300 mL EA and 250 mL saturated ammonium chloride were added into the reaction solution, the EA layer was separated, washed with saturated sodium chloride once, dried over anhydrous sodium sulfate, concentrated purified by silica gel column chromatography with gradient elution (DCM:MeOH=100:1-50:1). The product eluent was collected, condensed and dried to obtain 35 g solid. MS (ESI) m/z 552.36 ([M+H]⁺).

### Synthesis of Compound H-07

26 g compound H-06 was dissolved in 260 mL DCM, solution cooled by ice water bath, and 45 mL trifluoroacetic acid was added dropwise t. After dropping, the reaction was raised to room temperature and stirred for 1 h, and UPLC-MS was used to monitor the reaction complete. TFA was concentrated, and 200 mL DCM was added to the residue, saturated sodium bicarbonate added dropwise to adjust the pH to 8-9, DCM layer separated, washed with saturated sodium chloride, dried over anhydrous sodium sulfate, filtered, concentrated and dried to obtain 21.89 g oil. MS (ESI) m/z 452.30 ([M+H]⁺).

### Synthesis of Compound H-08

17.53 g compound M-3 was dissolved in 105 mL DMF, 21.21 g HBTU and 12.2 mL DIEA were added under the nitrogen atmosphere, and stirred at room temperature for 30 min. Then 100 mL DMF solution of 21 g compound H-07 was dropped in 20 min. Stirred at room temperature for 1 h, samples obtained was checked by TLC to monitor the reaction complete. 200 mL EA and 500 mL saturated sodium chloride were added to the reaction solution, separate organic phase, washed with saturated sodium chloride, dried over anhydrous sodium sulfate, concentrated, purified by silica gel column chromatography with gradient elution (PE:EA=10:1-3:1). The product eluent was collected and condensed, dried to obtain 20.2 g oily substance. MS (ESI) m/z 776.50 ([M+H]⁺).

### Synthesis of Compound H-09

19.66 g compound H-08 was dissolved in 50 mL THF, 100 mL methanol was added, and dissolved by stirring at room temperature. 3.2 g lithium hydroxide was added, and stirred at room temperature for 2 h. TLC was used to monitor the reaction complete. Methanol and THE were removed under reduced pressure. 100 mL ethyl acetate was added to the concentrated residue, and 2 M HCl was used to adjust pH to 3-4. Washed with saturated sodium chloride, dried with anhydrous sodium sulfate, and concentrated and dried to obtain 13 g white solid compound H-09. ¹H-NMR (400 MHz, DMSO-d6) δ7.85 (d, 1H), 7.52-7.65 (m, 9H), 7.34-7.47 (m, 12H), 6.77 (s, 1H), 4.72 (s, 2H), 4.20-4.24 (m, 1H), 4.09-4.14 (m, 1H), 3.53-3.59 (m, 2H), 2.80 (dd, 1H), 2.63 (dd, 1H), 2.39 (dd, 1H), 2.24 (dd, 1H), 9.67 (d, 21H). MS (ESI) m/z 762.40 ([M+H]⁺).

### Step 2: The Synthesis Route of Compound HC-13

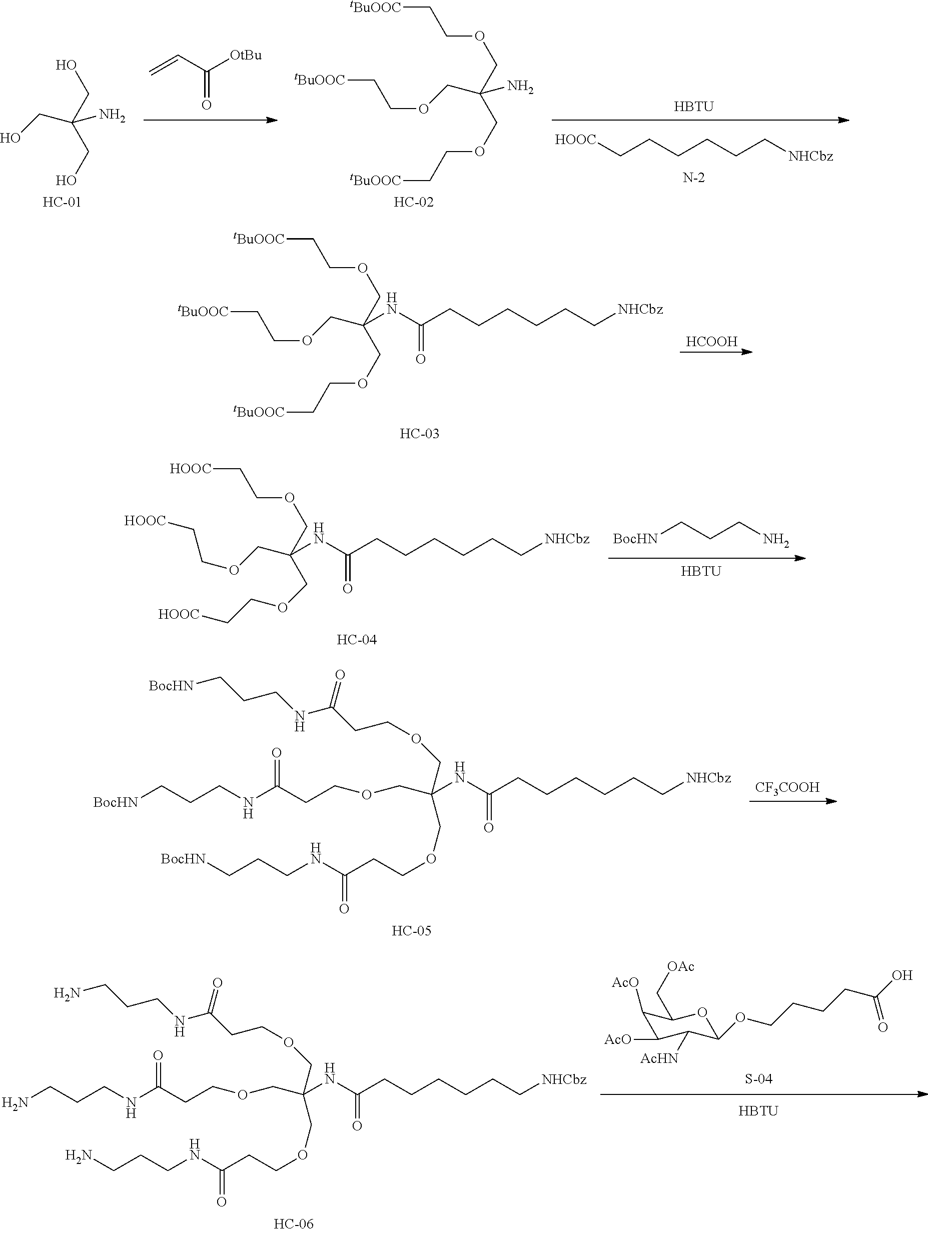

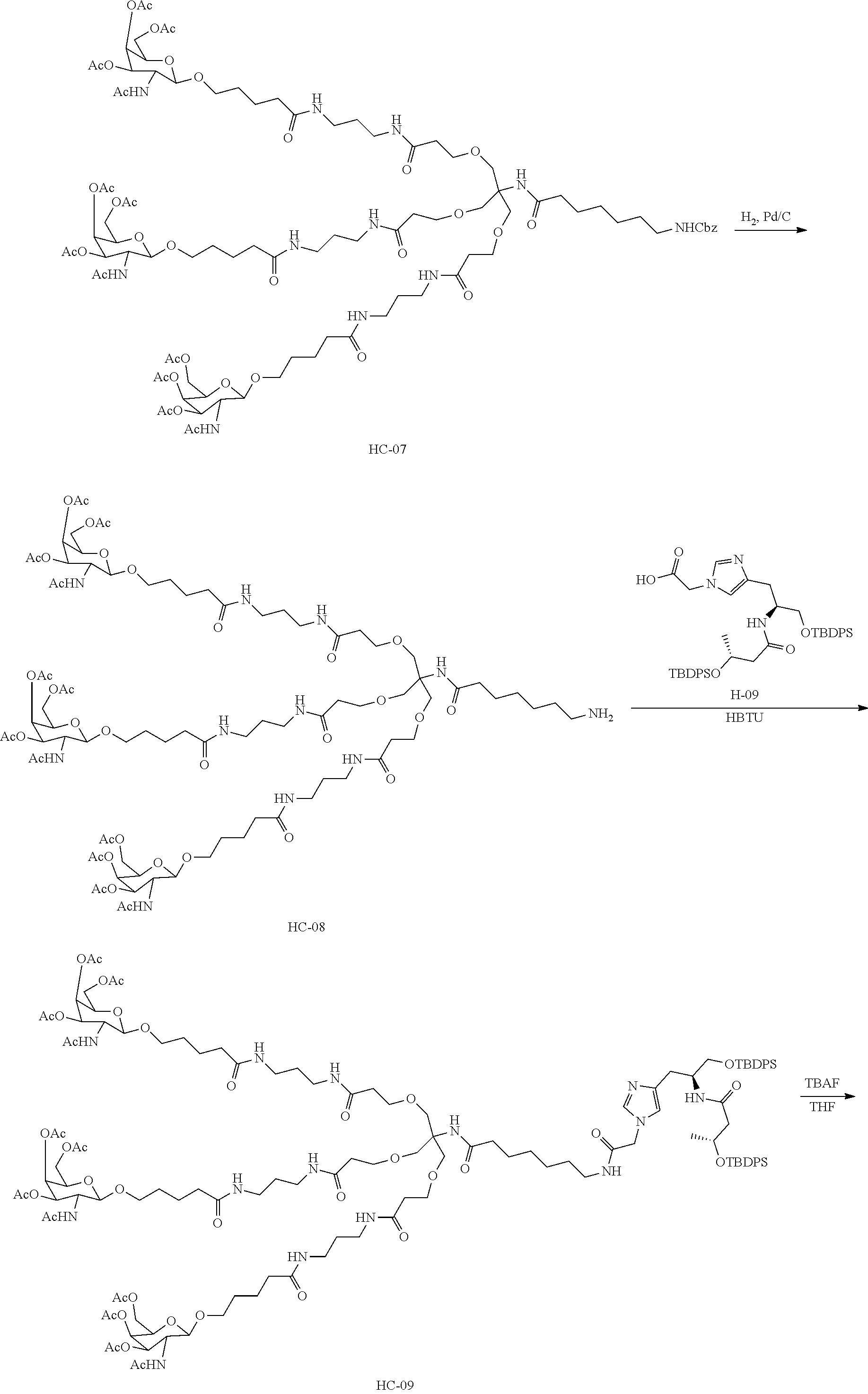

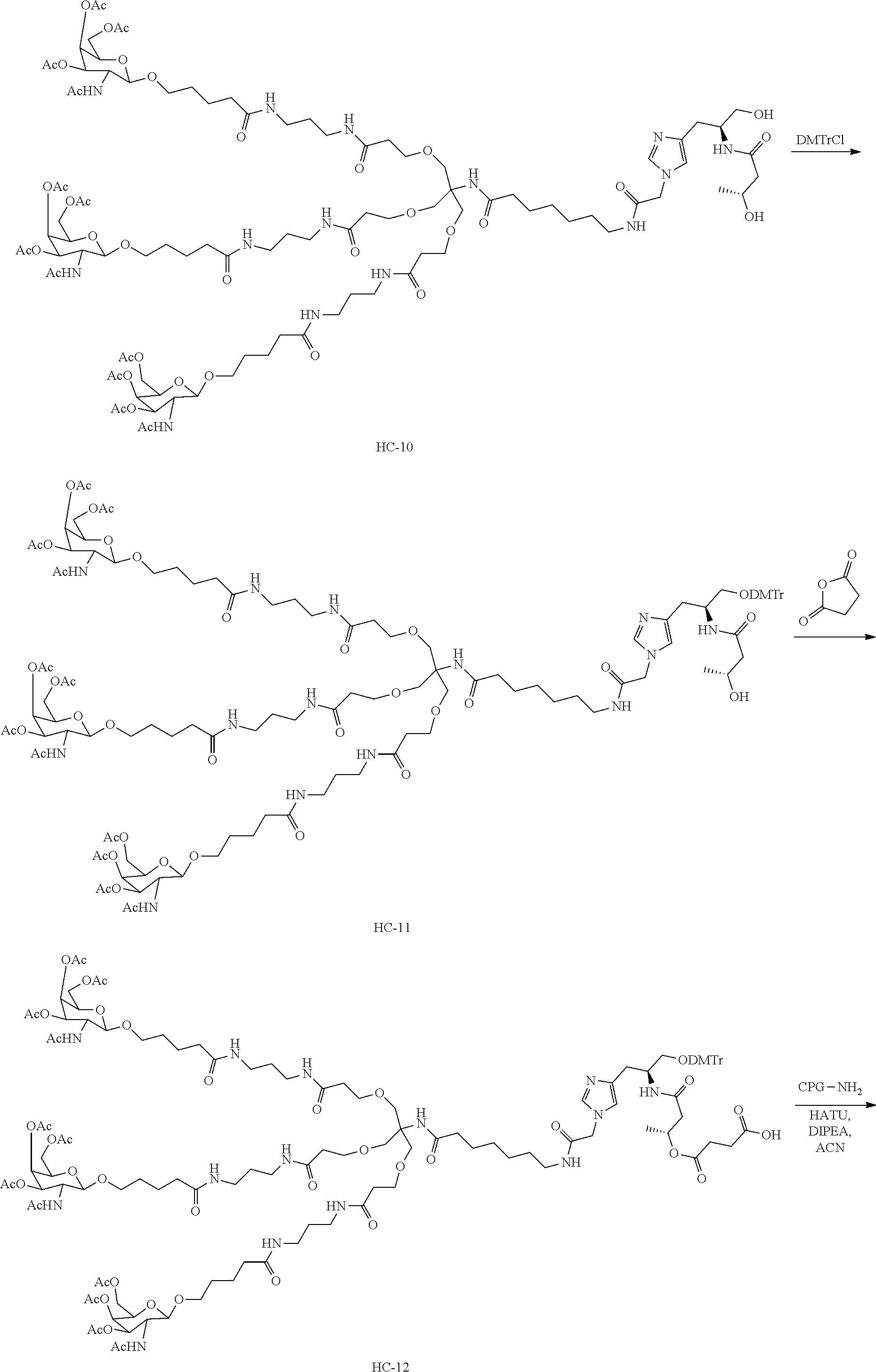

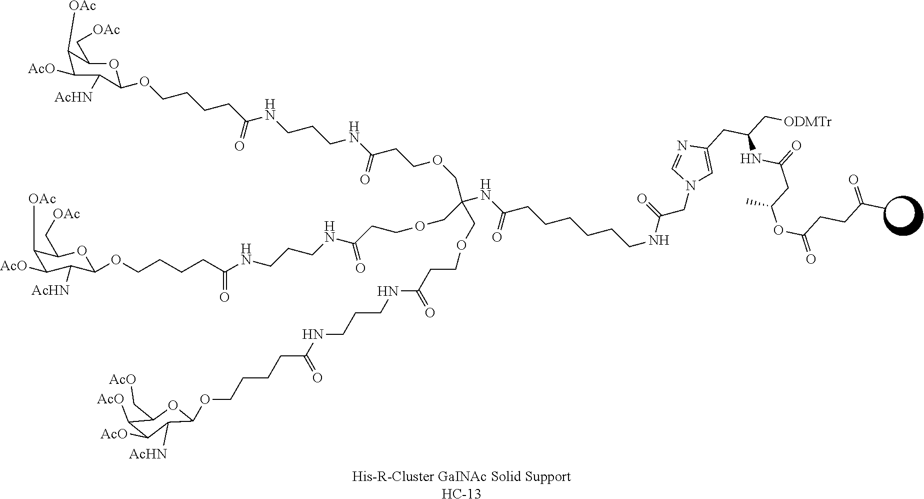

The synthesis method of compound HC-02 to HC-08 is the same as the synthesis route in GC-05 (Glu(R)-Cluster GalNAc).

### Synthesis of Compound HC-09

3.9 g compound H-09 was dissolved in 40 mL DMF, 2.92 g HBTU and 2 g DIPEA were added, and stirred at room temperature for 15 minutes. 11.5 g intermediate compound HC-08 was dissolved in 100 mL DMF, and dropped slowly to the above system by stirring, stirred at room temperature for 30 minutes. TLC was used to monitor the reaction complete. 100 mL ethyl acetate and 100 mL saturated sodium bicarbonate were added to the reaction solution, and EA layer was extracted. The organic phase was washed with saturated sodium chloride, dried over anhydrous sodium sulfate, concentrated, and purified by silica gel column chromatography (Dichloromethane:methanol=50:1-10:1). The product eluent was collected and dried under reduced pressure to obtain 10.5 g white solid. MS (ESI) m/z 889.44 ([(M+3)/3]⁺).

### Synthesis of Compound HC-10

s10.1 g compound HC-09 was dissolved in 150 mL THF, and 11.4 mL TBAF (1 M in THF) was added, and stirred overnight at room temperature. After the reaction was complete monitored by LC-MS, the solvents were removed by distillation under reduced pressure, and the crude product was purified by silica gel column chromatography with gradient elution (methylene chloride:methanol=50:1-10:1). The product eluent was collected, condensed and dried to obtain 7.53 g white solid. MS (ESI) m/z 1094.94 ([(M+2)/2]⁺).

### Synthesis of Compound HC-11

3.9 g compound HC-10 was dissolved in 33 mL anhydrous pyridine, and 1.03 g DMTrCl was added to the solution by stirring. After stirring at room temperature for 10 minutes, TLC was used to monitor the reaction complete. The reaction was quenched by 12 mL methanol, solvent removed by distillation under reduced pressure. Purified by silica gel column chromatography through wet packing with gradient elution (dichloromethane:methanol=50:1-20:1). The product eluent was collected, condensed and dried to obtain 3.62 g pale yellow solid. MS (ESI) m/z 1094.95 ([(M-302)+2)/2]⁺).

### Synthesis of Compound HC-12

3.6 g compound HC-11 was dissolved in 36 mL anhydrous pyridine, 20 mg DMAP and 2.9 g succinic anhydride were added, stirred overnight at room temperature under nitrogen atmosphere. TLC was used to monitor the reaction complete. Pyridine was removed by distillation under reduced pressure and purified by silica gel column chromatography (Dichloromethane:methanol=50:1-10:1). Product eluent was collected, condensed and dried to obtain 2 g white solid. ¹HNMR (400 MHz, DMSO-d6) δ 8.02-7.92 (m, 10H), 7.43-7.38 (m, 3H), 7.31-7.20 (m, 8H), 7.06 (s, 1H), 6.88-6.86 (m, 4H), 6.64 (s, 1H), 5.20 (d, 3H), 4.98 (dd, 3H), 4.55-4.49 (m, 5H), 4.20-4.15 (m, 2H), 4.05-3.97 (m, 8H), 3.87 (q, 3H), 3.73 (s, 6H), 3.68-3.73 (m, 3H), 3.55-3.52 (m, 12H), 3.41-3.39 (m, 4H), 3.04-3.01 (m, 14H), 2.90 (d, 2H), 2.77-2.59 (m, 4H), 2.30-2.26 (m, 8H), 2.10 (s, 9H), 2.06-2.03 (m, 8H), 1.99 (s, 9H), 1.88 (s, 9H), 1.77 (s, 9H), 1.52-1.43 (m, 20H), 1.27-1.20 (m, 9H), 1.11 (d, 3H), MS (ESI) m/z 1145.05 ([(M-302)+2)/2]⁺).

### Synthesis of Compound HC-13

236 mg compound HC-12 was dissolved in 10 mL acetonitrile, 49.6 mg HATU and 29.8 mg DIPEA were added, and the mixture was shaken in a shaker for 10 minutes. 800 mg LCAA-CPG (96 μmol/g) was added to the reaction solution and shaked overnight. The next day, the reaction solution was filtered and the filter cake was washed with acetonitrile. 5 mL CapA (20% NMI-80% CAN) and 5 mL CapB (20% AC₂O-30% lutidine-50% CAN) were added to the filter cake. Mixture was continue to be shake at room temperature for 2 hours in a shaker, reaction solution filtered, and the filter cake was washed with acetonitrile, dried under vacuum at room temperature for 2 hours to obtain 780 mg compound HC-13 with 46 μmol/g loading.

### Example 3 Synthesis of B-9 (His(R)-Cluster GalNAc)

Synthesis of B-9 (His(R)-Cluster GalNAc) was show below (2 steps):

### Step 1: The Synthesis Route of Compound Y-03

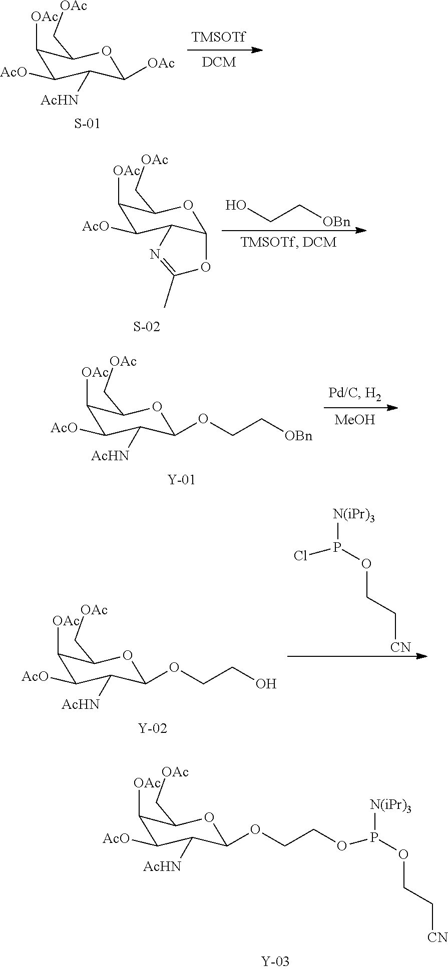

### Synthesis of Compound Y-01

Under the nitrogen atmosphere, 169 g compound S-02 was dissolved in 1.2 L anhydrous DCM, 83.6 g 1-benzoxy ethanol was added, 57.38 g TMSOTf was dropped, and the reaction was carried out overnight at room temperature. TLC was used to monitor the reaction complete, cooled in ice water bath, saturated NaHCO₃ was dropped to adjust pH to 7-8, and then the organic phase was extracted and separated. The organic phase was washed with saturated NaCl once, dried with anhydrous sodium sulfate, filtered, and the filtrate was concentrated and dried, purified by column chromatography to obtain 182 g compound Y-01. MS (ESI) m/z 482.16 ([M+H]⁺).

### Synthesis of Compound Y-02

80 g compound Y-01 was dissolved in 600 mL methanol, and 8 g 10% Pd/C was added. After replaced the air with hydrogen for 3 times, the reaction was completed at room temperature for 10 h stirring. Palladium carbon was filtered out and the filtrate was concentrated and dried to obtain 59.86 g gray solid. MS (ESI) m/z 392.19 ([M+H]⁺).

### Synthesis of Compound Y-03

Under the nitrogen atmosphere, compound Y-02 (50.0 g) was dissolved in 500 mL anhydrous CH₂Cl₂, then 45 mL DIPEA was added, and 40 g N, N-diisopropyl chlorophosphoride was added, and reacted by stirring at room temperature for 2 h. TLC was used to monitor reaction complete, then concentrated, separate rapidly by column chromatography to obtain 46.02 g compound Y-03. MS (ESI) m/z 509.19 ([M+H]⁺).

### Step 2: The Synthesis Route of Compound B-9

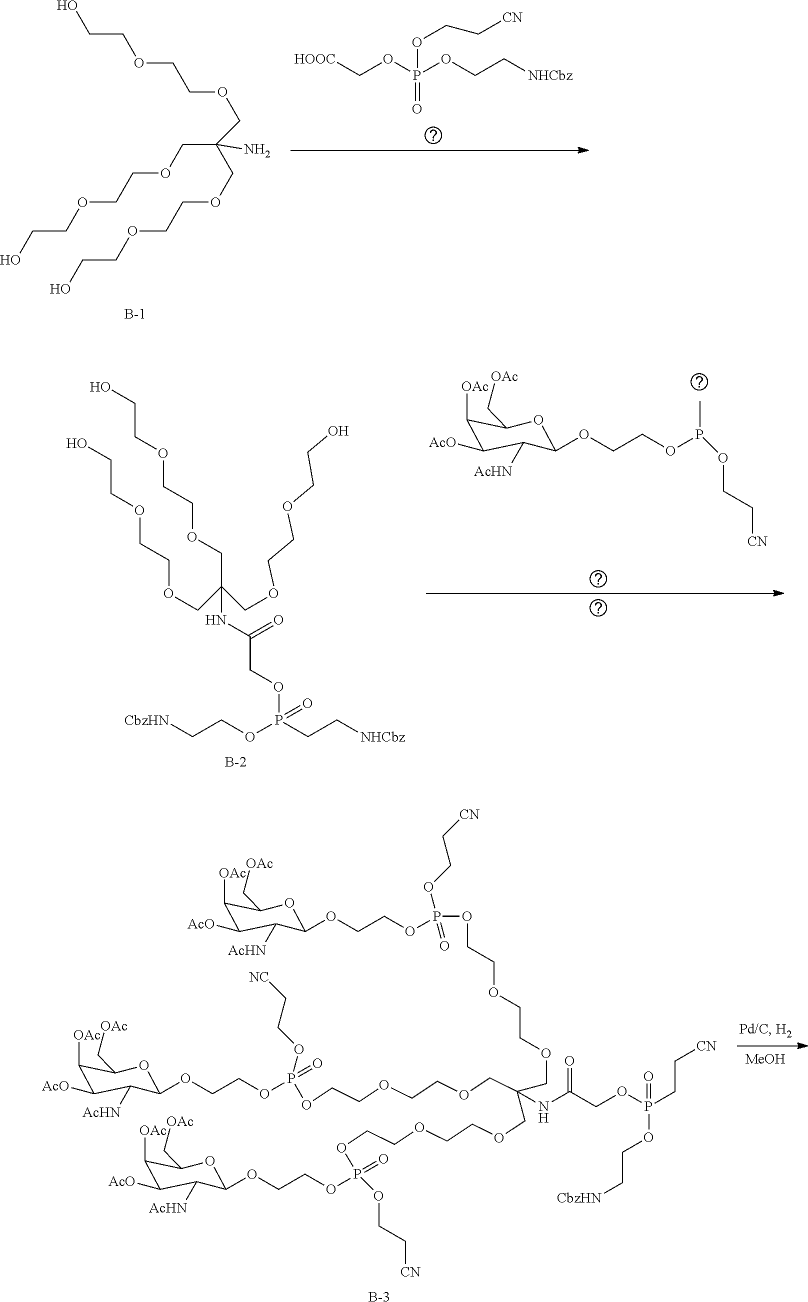

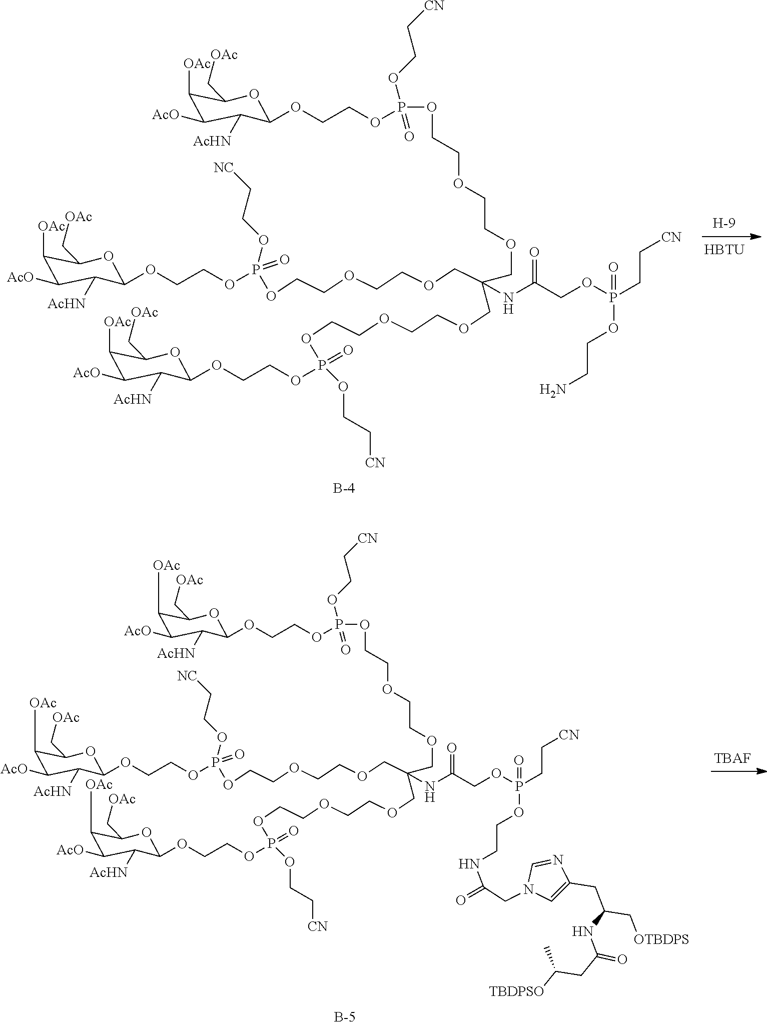

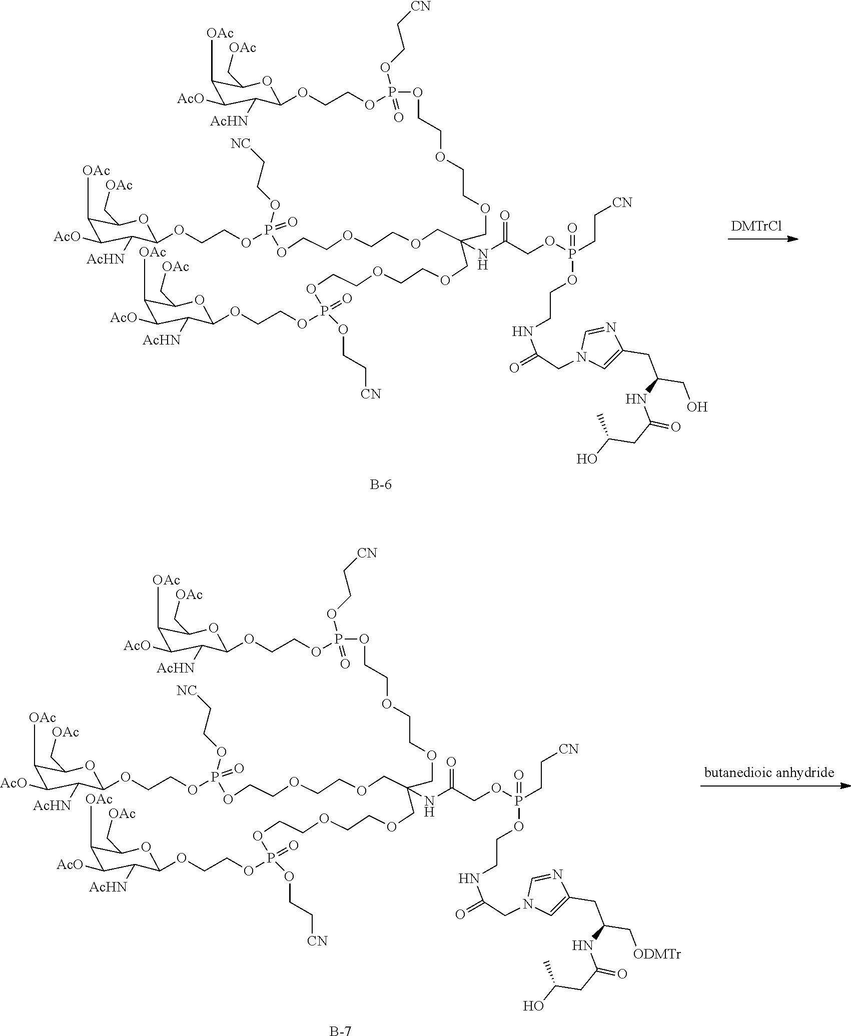

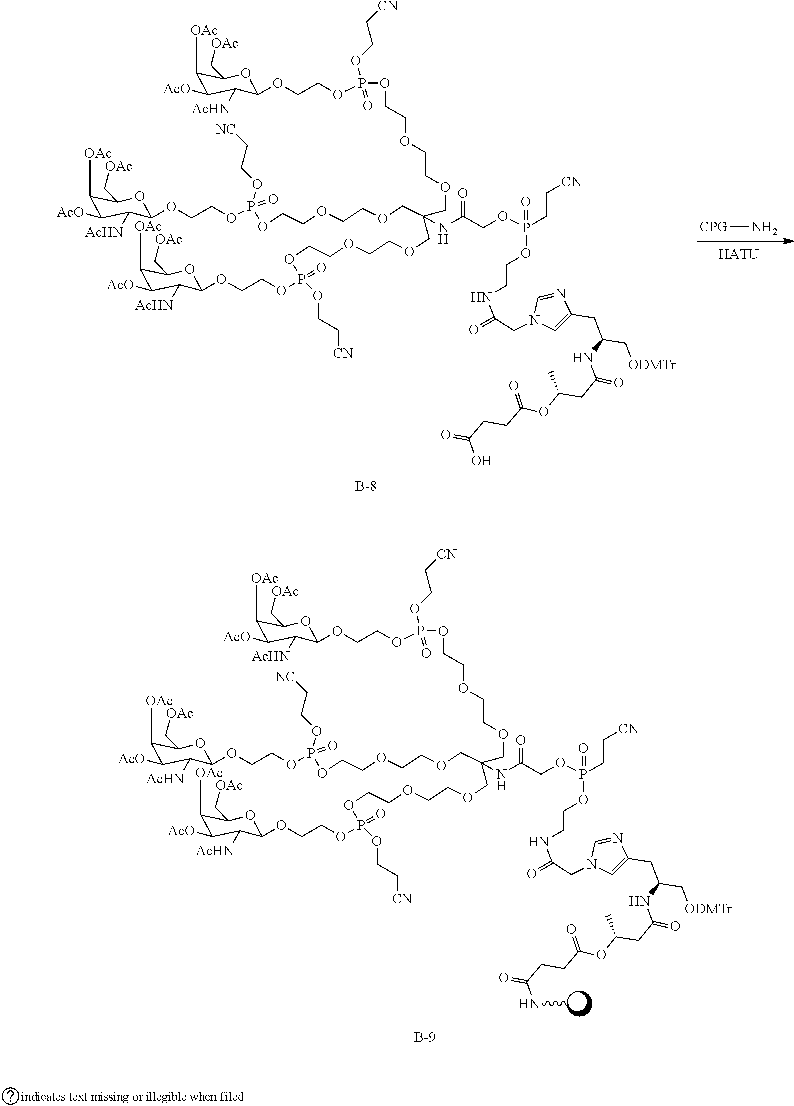

### Synthesis of Compound B-2

20.24 g compound SM-2 (purchased from PharmaBlock Sciences (Nanjing), Inc.) was dissolved in 200 mL DMF, then 15.26 g DIPEA and 30.13 g HBTU were added, and stirred at room temperature for 20 minutes. 20.02 g compound B-1 (purchased from PharmaBlock Sciences (Nanjing), Inc.) dissolved in 50 mL DMF was added dropwise to the reaction solution. After dropping, TLC was used to monitor the reaction complete after stirring at room temperature for 1 hour. 600 mL saturated sodium bicarbonate was added to the reaction solution, then extracted with 300 mL ethyl acetate, washed with saturated salt water, dried over anhydrous sodium sulfate, concentrated, purified by silica gel column with gradient elution (petroleum ether:ethyl acetate). The product eluent was collected, concentrated and dried to obtain 33.20 g compound B-2. MS (ESI) m/z 738.40 ([M+H]⁺).

### Synthesis of Compound B-3

33 g compound B-2 was dissolved in 400 mL 50% CH₂Cl₂/acetonitrile, 180 mL activator (1 M 4,5-dicyanimidazole/1% N-methylimidazole) was added to the system, and 90 g Y-03 dissolved in 100 mL acetonitrile was added dropwise to the system. After stirring at room temperature for 30 min, 160 mL tert-butyl hydrogen peroxide/acetonitrile/water (10:87:3) was added to the reaction solution, and TLC was used to monitor the reaction complete. The solvent was removed by distillation under reduced pressure and purified by silica gel column chromatography to obtain 77.45 g compound B-3. MS (ESI) m/z 1128.79 ([(M+2)/2]⁺).

### Synthesis of Compound B-4

60 g compound B-3 was dissolved in 600 mL methanol, and 6 g 10% Pd/C was added. After with hydrogen for 3 times, and the reaction was complete after stirred at room temperature for 8 h. Palladium carbon was filtered and the filtrate was concentrated and dried to obtain 51.54 g gray solid. MS (ESI) m/z MS (ESI) m/z 1061.74 ([(M+2)/2]⁺).

### Synthesis of Compound B-5

16 g compound H-09 was dissolved in 160 mL DMF, 11.6 g HBTU and 5.9 g DIPEA were added, stirred at room temperature for 15 minutes. 46 g of intermediate compound B-4 was dissolved in 400 mL DMF, then the solution was slowly dropped to the above system by stirring, stirred at room temperature for 30 minutes, and UPLC-MS was used to monitor the reaction complete. 400 mL ethyl acetate and 400 mL saturated sodium bicarbonate were added to the reaction solution, and EA layer was extracted, washed with saturated sodium chloride, dried over sodium sulfate, and purified by silica gel column chromatography. Eluent of the product was collected, condensed and dried to obtain 42.1 g white solid. MS (ESI) m/z 956.20 ([(M+3)/3]⁺).

### Synthesis of Compound B-6

40.0 g intermediate compound B-5 was dissolved in 600 mL anhydrous THF, and 45.6 mL TBAF (1 M in THF) was added at room temperature by stirring, and stirred overnight. UPLC-MS was used to monitor the reaction complete. The solvents were removed by under reduced pressure, and the crude product was eluted by silica gel column chromatography with gradient elution (dichloromethane/methanol). The eluent of the product was collected, condensed, and dried to obtain 23.10 g white solid. MS (ESI) m/z 797.28 ([(M+3)/3]⁺).

### Synthesis of Compound B-7

Under nitrogen atmosphere, compound B-6 (30.0 g) was dissolved in 300 mL anhydrous pyridine, 6.2 g DMTrCl was added and stirred for 30 min at room temperature. TLC was used to monitor the reaction complete, concentration, and silica gel column chromatography separation were conducted, to obtain white crystalline solid (23.02 g), MS (ESI) m/z 797.38 ([(M-302+3)/3]⁺).

### Synthesis of Compound B-8

Under nitrogen atmosphere, 10 g B-7 was dissolved in 100 mL anhydrous pyridine, 100 mg DMAP and 5.6 g succinic anhydride were added, stirred at room temperature for 72 h. After the reaction completed, pyridine was concentrated and the product compound B-8 was purified by silica gel column chromatography to obtain 7.46 g. MS (ESI) m/z 830.33 ([(M-302+3)/3]⁺).

### Synthesis of Compound B-9

3 g compound B-8 was dissolved in acetonitrile 30 mL, 650 mg HATU and 300 mg DIEA were added, shaken at room temperature for 10 min, 10 g CPG-NH₂ (96 umol/g) was added. then continued to be shake overnight. Filtration, and leaching by acetonitrile were conducted, capA 50 mL and capB 50 mL were added to filter cake, then continued to be shake for 2 h. Filtration, and leaching by acetonitrile were conducted, and dried under vacuum for 3 h, 11.2 g compound B-9 with 45 μmol/g loading was obtained.

### Example 4 Synthesis of SC-13 (His(R)-Cluster GalNAc)

Synthesis of SC-13 (His(R)-Cluster GalNAc) was show below:

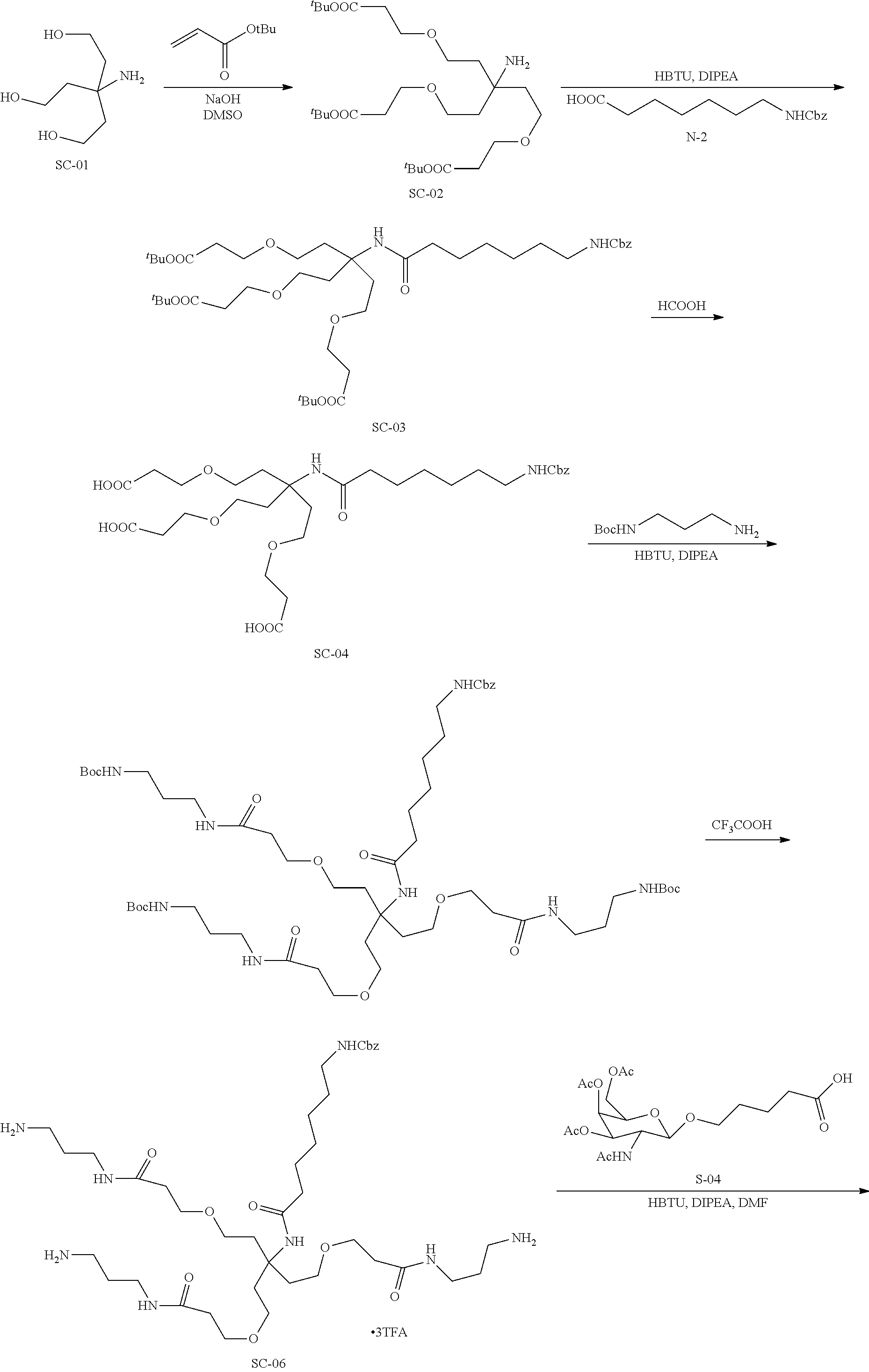

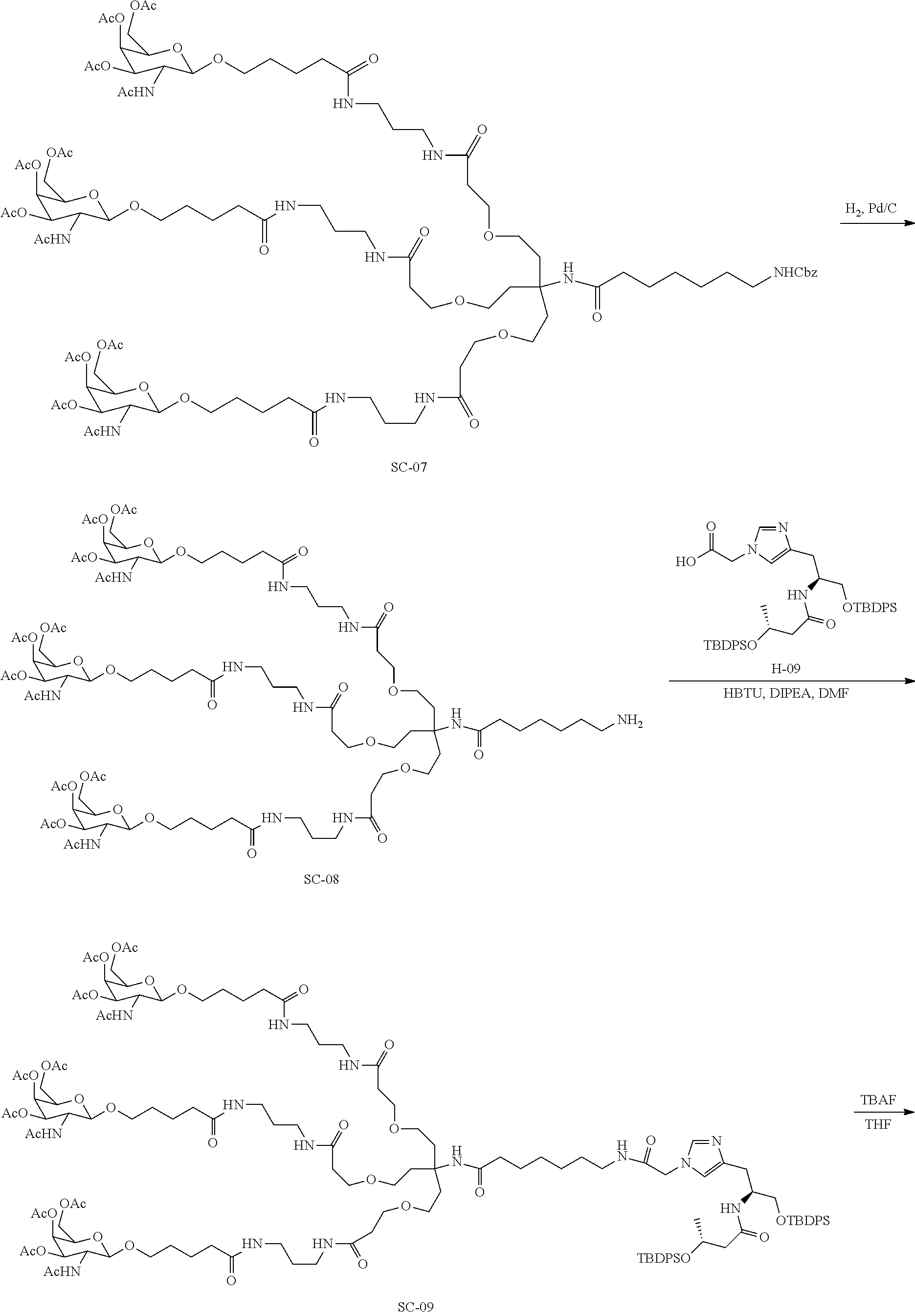

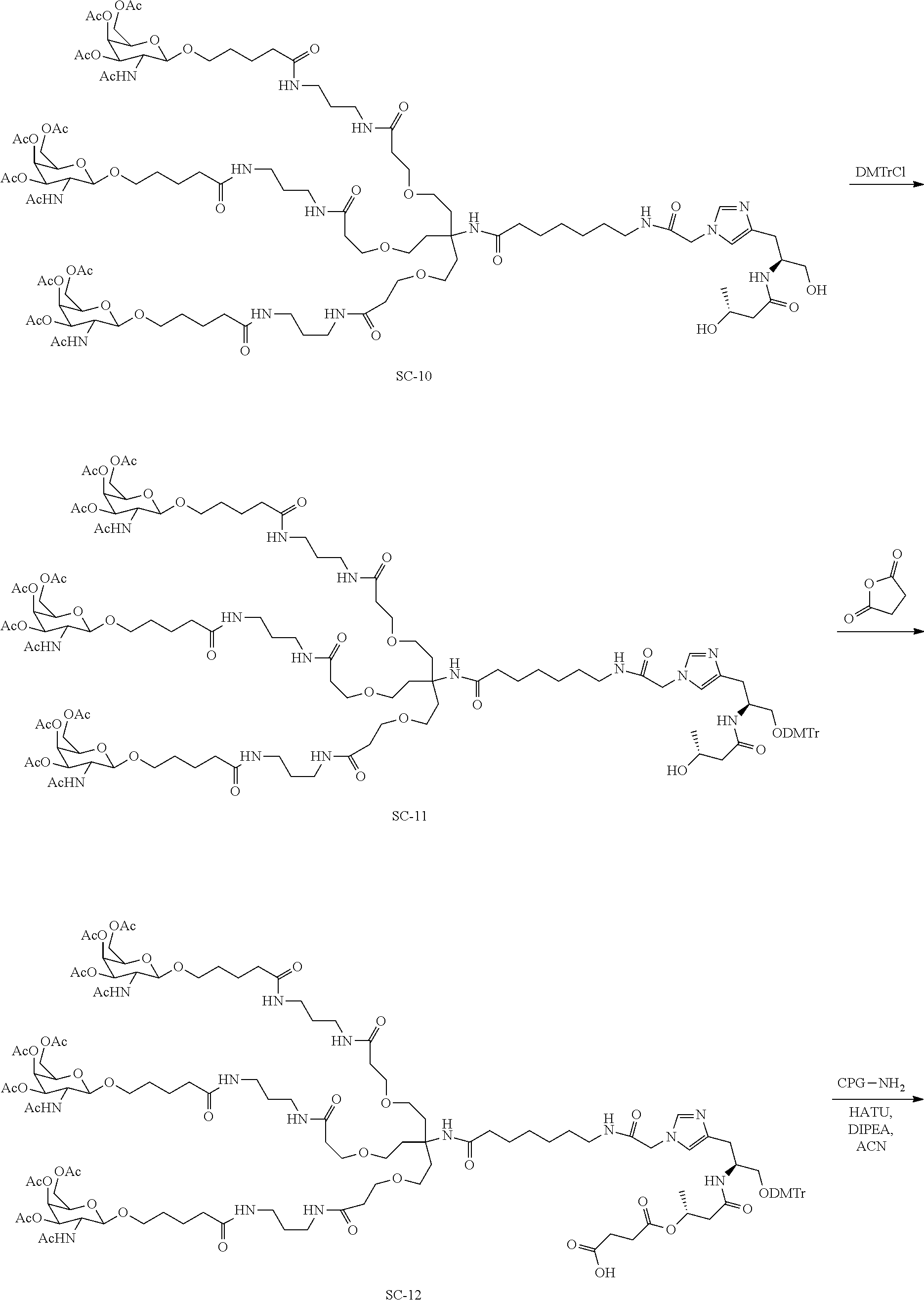

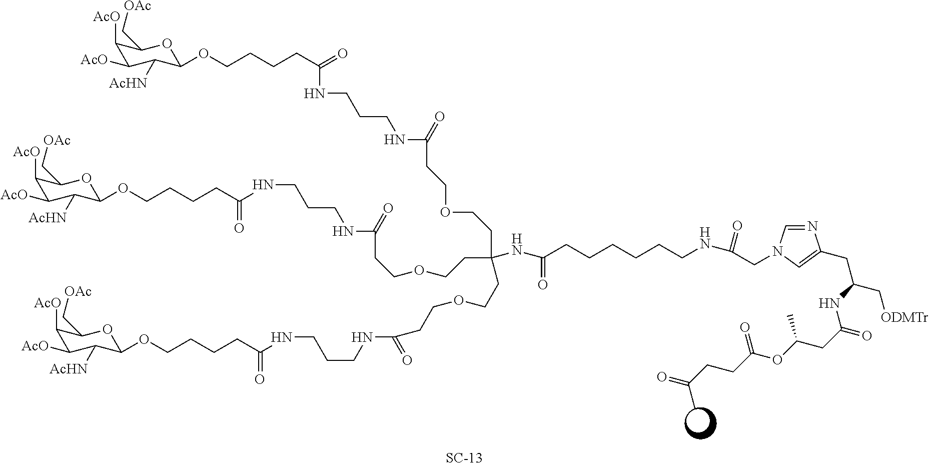

### Synthesis of Compound SC-02

The starting material was replaced with 3-amino-3-(2-hydroxyethyl)-1,5-pentanediol, and the synthesis method was the same as compound HC-02. MS (ESI) m/z 548.35 ([M+H]⁺).

### Synthesis of Compound SC-03

The synthesis method of it is the same as compound HC-03 in His(R)-Cluster GalNAc synthesis route. MS (ESI) m/z 809.66 ([M+H]⁺)

### Synthesis of Compound SC-04

The synthesis method of it is the same as compound HC-04 in His(R)-Cluster GalNAc synthesis route. MS (ESI) m/z 639.30 ([M−H]⁻).

### Synthesis of Compound SC-05

The synthesis method of it is the same as compound HC-05 in His(R)-Cluster GalNAc synthesis route. MS (ESI) m/z 1109.16 ([M+H]⁺).

### Synthesis of Compound SC-06

The synthesis method of it is the same as compound HC-06 in His(R)-Cluster GalNAc synthesis route. MS (ESI) m/z 809.47 ([M+H]⁺).

### Synthesis of Compound SC-07

The synthesis method of it is the same as compound HC-07 in His(R)-Cluster GalNAc synthesis route. MS (ESI) m/z 1049.23 ([(M+2)/2]⁺).

### Synthesis of Compound SC-08

The synthesis method of it is the same as compound HC-08 in His(R)-Cluster GalNAc synthesis route. MS (ESI) m/z 982.09 ([(M+2)/2]⁺).

### Synthesis of Compound SC-09

The synthesis method of it is the same as compound HC-09 in His(R)-Cluster GalNAc synthesis route. MS (ESI) m/z 902.62 ([(M+3)/3]⁺).

### Synthesis of Compound SC-10

The synthesis method of it is the same as compound HC-10 in His(R)-Cluster GalNAc synthesis route. MS (ESI) m/z 1115.52 ([(M+2)/2]⁺).

### Synthesis of Compound SC-11

The synthesis method of it is the same as compound HC-11 in His(R)-Cluster GalNAc synthesis route. MS (ESI) m/z 1115.56 ([(M-302)+2)/2]⁺).

### Synthesis of Compound SC-12

The synthesis method of it is the same as compound HC-12 in His(R)-Cluster GalNAc synthesis route. MS (ESI) m/z 1165.62 ([(M-302)+2)/2]⁺). ¹HNMR (400 MHz, DMSO-d6) δ 8.01-7.88 (m, 10H), 7.40-7.34 (m, 3H), 7.29-7.18 (m, 8H), 7.06 (s, 1H), 6.78-6.82 (m, 4H), 6.60 (s, 1H), 5.18 (d, 3H), 4.96 (dd, 3H), 4.54-4.46 (m, 5H), 4.20-4.15 (m, 2H), 4.05-3.93 (m, 8H), 3.82 (q, 3H), 3.70 (s, 6H), 3.65-3.71 (m, 3H), 3.55-3.50 (m, 12H), 3.41-3.34 (m, 4H), 3.04-3.01 (m, 14H), 2.87 (d, 2H), 2.77-2.56 (m, 4H), 2.30-2.26 (m, 8H), 2.10 (s, 9H), 2.06-2.01 (m, 8H), 1.96 (s, 9H), 1.94-1.90 (m, 6H), 1.85 (s, 9H), 1.75 (s, 9H), 1.52-1.43 (m, 20H), 1.27-1.20 (m, 9H), 1.11 (d, 3H), MS (ESI) m/z 1145.05 ([(M-302)+2)/2]⁺).

### Synthesis of Compound SC-13

The synthesis method of it is the same as HC-13 in His(R)-Cluster GalNAc synthesis route.

### Example 5 Synthesis of Conjugated Oligonucleotide Provided by this Invention

A naturally occurring or chemically modified oligonucleotide was synthesized by a usual method, e.g. solid phase method. And the compound conjugated oligonucleotide was synthesized by exemplary method as shown below:

### Preparation of Oligo Conjugate GC-05 (Glu-R-Cluster GalNAc)

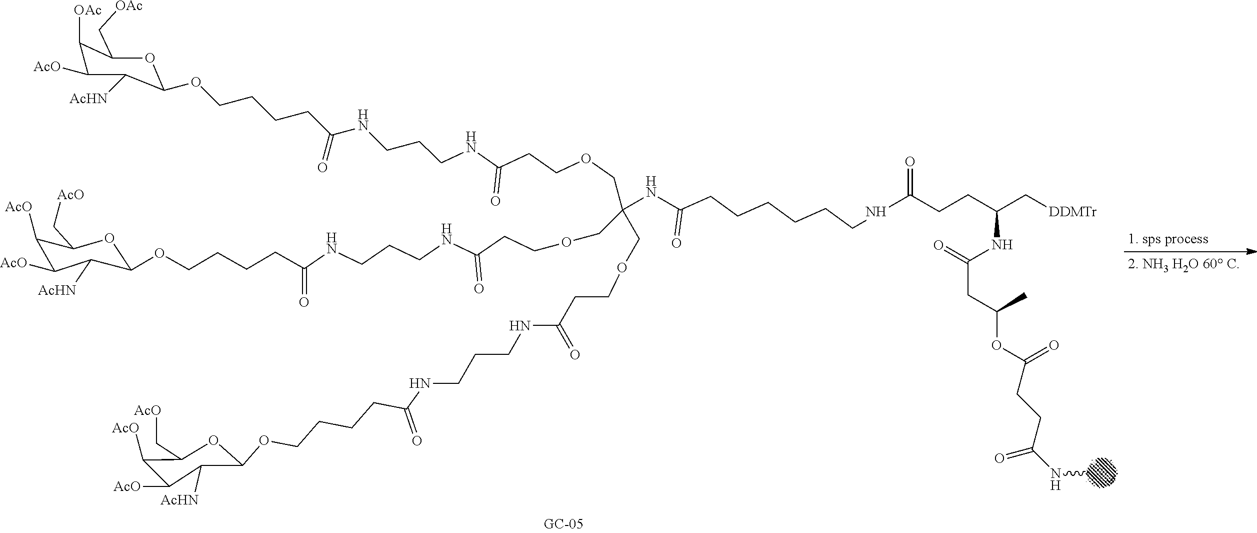

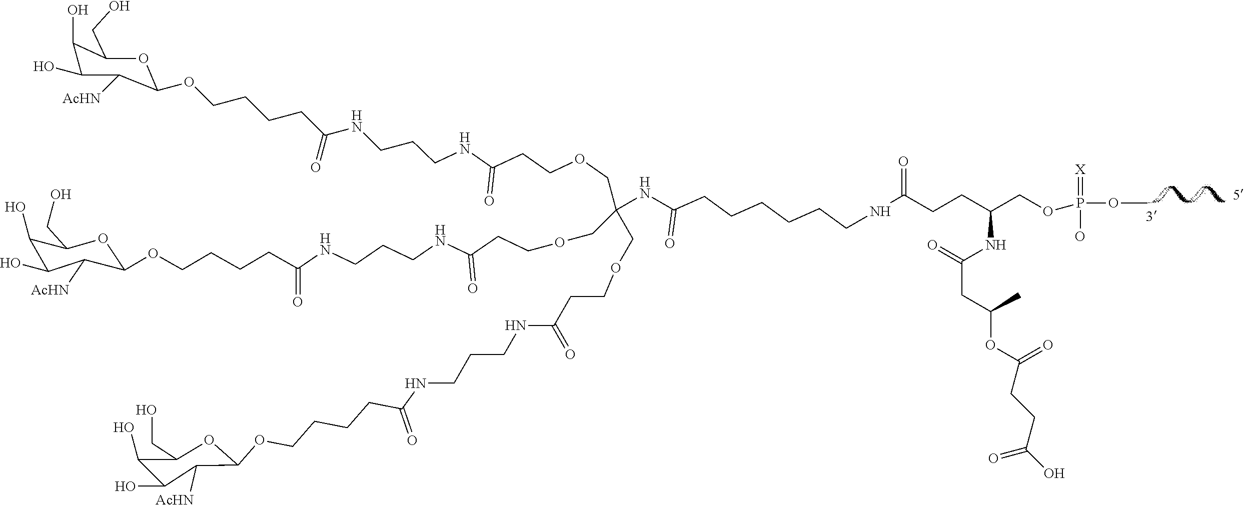

### Steps of Solid-Phase Synthesis

Using the phosphoramidite solid-phase synthesis method known in the art, GC-05 was used as the solid-phase synthesis carrier, and the nucleoside monomers were connected one by one in 3′ to 5′ direction in the sequence order by MerMade192 solid phase synthesizer. Each connection of a nucleoside monomer included four steps: deprotection, coupling, capping and oxidation, standard procedures of the steps mentioned-above are known to one of ordinary skills in the art, and all monomer solutions were prepared with 0.1 M acetonitrile solutions.

The solid phase synthesis reagents were configured as follows:
Wash: acetonitrile
Deblock: 3% Dichloro Acetic Acid in Dichloromethane
Activator: 0.25M 5-Ethylthio-1H-Tetrazole in Acetonitrile
Capping Reagent A: THF/Lutidine/Acetic Anhydride (8:1:1)
Capping Reagent B: 15% NMI/THF, GL38 finish
Oxidizing reagent: 0.02 M I2 in THF/Pyridine/H₂O
Sulfurizing reagent: 0.10 M DDT T solution

The solid-phase synthesis conditions taking 1 μmol synthesis scale as an example are as follows:

| PO cycle file | | PO Reagent file | | | |
|---|---|---|---|---|---|
| Number of | | Volume of | | Reaction | |
| steps | Parameter | reagent (μL) | | time (s) | |
| 3 × wash | Wash | 200 | μL | 0 | s |
| 2 × deblock | Deblock | 150 | μL | 45 | s |
| 2 × wash | Wash | 200 | μL | 0 | s |
| 2 × coupling | Activator | 85 | μL | 360 | s |
| | Amidite | 70 | μL | | |
| 1 × wash | Wash | 200 | μL | 0 | s |
| 1 × capping | Cap A | 75 | μL | 60 | s |
| | Cap B | 75 | μL | | |
| 1 × wash | Wash | 200 | μL | 0 | s |
| 1 × oxidizing | Oxidizer | 150 | μL | 60 | s |
| 2 × wash | Wash | 200 | μL | 0 | s |
| PS cycle file | | PS Reagent file | | | |
| Number of | | Volume of | | Reaction | |
| steps | | reagent (μL) | | time (s) | |
| 3 × wash | Wash | 200 | μL | 0 | s |
| 2 × deblock | Deblock | 150 | μL | 45 | s |
| 2 × wash | Wash | 200 | μL | 0 | s |
| 2 × coupling | Activator | 85 | μL | 360 | s |
| | Amidite | 70 | μL | | |
| 1 × wash | Wash | 200 | μL | 0 | s |
| 1 × sulfurizing | Sulfurizer | 200 | μL | 200 | s |
| 2 × wash | Wash | 200 | μL | 0 | s |
| 1 × capping | Cap A | 75 | μL | 60 | s |
| | Cap B | 75 | μL | | |
| 2 × wash | Wash | 200 | μL | 0 | s |

### Steps of Cleavage and Deprotection

The Oligo-support obtained by the steps of solid-phase synthesis above was added to a 1 mL centrifuge tube, 50˜100 μL of concentrated ammonium hydroxide was added, cultured at 50-60° C. for 10 hours, the liquid supernatant was drawn by centrifugation, two-fold volume acetone-ethanol (80:20) solvent was added to the liquid supernatant, a white precipitate was precipitated, and the supernatant was removed by centrifugation at 10,000 g to obtain a precipitated product, the precipitate was redissolved in 0.2 M sodium acetate solution.

### Steps of Purification, Desalting and Lyophilization

Using ion chromatography column which has 1 mL volume (loaded with packing Nano Q 30), purification was carried out on Avant 150 purification equipment.

The detailed conditions are:
Buffer A: 20 mM sodium phosphate-10% acetonitrile-water buffer solution (pH7.5),
Buffer B: 2.0 M NaCl-20 mM sodium phosphate-acetonitrile-water buffer solution (pH7.5);
elution gradient: Buffer B 0˜50%, Buffer A 100˜50%

The eluates was collected and combined, and G25 Sephadex column was used for desalting finally; measured the OD 260 concentration value of the desalted product solution, the product content was calculated, and finally put it into a centrifuge tube for lyophilization to obtain a white freeze-dried product.

Detection: Reversed-phase UPLC-MS tandem mass spectrometry was used for detection, the purity was above 90%, and m/z [M-7/7]⁻, [M-8/8]⁻, [M-9/]⁻ characteristic ion peaks was showed on the mass spectrometry. For the synthesis example of AS-Oligo, refer to GC-05-OLIGO, and Unylinker-CPG (From Glen Research) was used as a solid-phase synthesis carrier for synthesis.

For the synthesis example of Oligo conjugate His-R-Cluster GalNAc, refers to GC-05-OLIGO, and HC-13 was used as solid phase synthesis carrier for synthesis.

For the synthesis example of Oligo conjugate His-R-Cluster GalNAc, refers to GC-05-OLIGO, and SC-13 was used as solid-phase synthesis carrier for synthesis.

For the synthesis example of Oligo conjugate His-R-Cluster GalNAc, refers to GC-05-OLIGO, and B-9 was used as solid-phase synthesis carrier for synthesis.

siRNA-GalNAc conjugate or aiRNA-GalNac conjugate was prepared by annealing Oligo-GalNAc conjugate obtained above and anti-sense strand with its complementary sequence at a molar ratio of 1:1 to obtain a double-stranded product.

### Activity Test

### Materials and Method

Conjugated aiRNA Synthesis for Test:

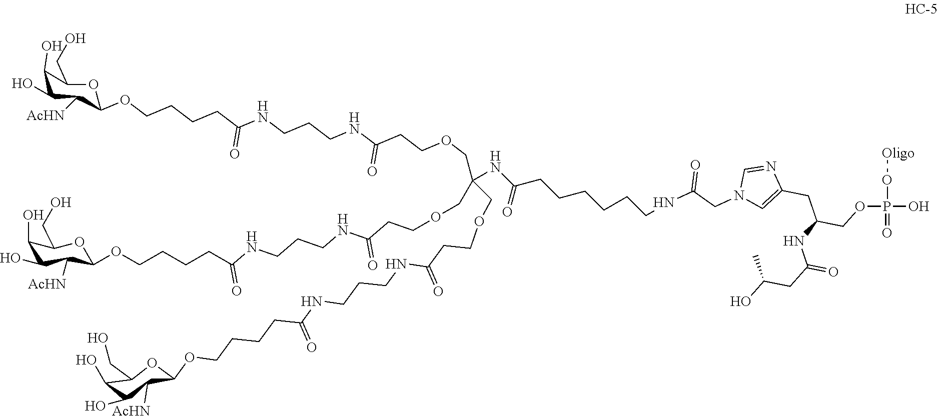

Our designed compound HC-5 is a triple Cluster GalNAc based on Histidine linker conjugated at 3′ end of sense strand of a duplex RNA such as aiRNA or siRNA, it is represented by code “His-cluster” as used and described in below examples.

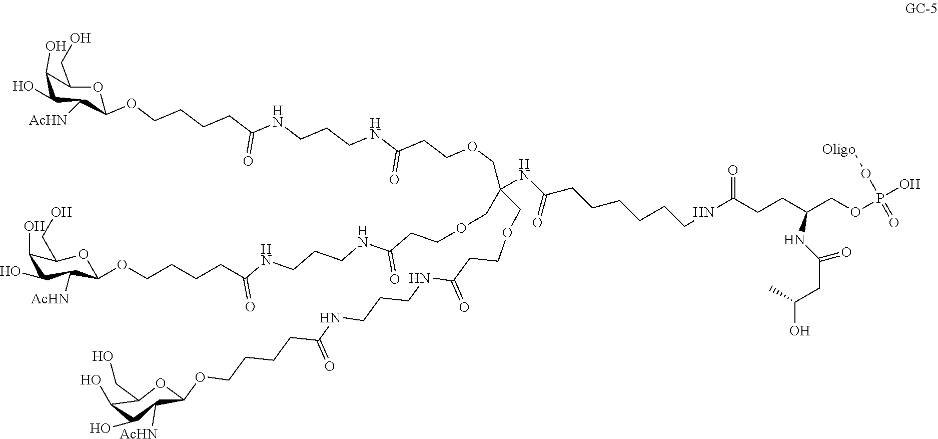

Our designed compound GC-5 is a triple Cluster GalNAc based on Glutamine linker conjugated at 3′ end of sense strand of a duplex RNA such as aiRNA or siRNA, it is represented by code “Glu-cluster” as used and described in below examples.

Sequences and structure of oligonucleotide (aiRNA and siRNA) synthesized and used in activity test examples are showed in below table:

sequence of tested aiRNA

| | Sense Strand | SEQ ID | Antisense strand | SEQ ID | |
|---|---|---|---|---|---|
| aiRNA# | (5′-3′)(SS) | No. | (5′-3′) (AS) | No. | as/ss |
| 1 | A*G*UGgauucuGuaCUGU-L | SEQ | A*a*ACAgUacAGaAuCCACU*G*G | SEQ | 17/21 mer, |
| | | NO. 1 | | NO. 2 | SS middle |
| 2 | U*G*gauucuGuaCUGUUU-L | SEQ | | | 17/21 mer, |
| | | NO. 3 | | | SS-3′blunt |

AGCU represent 2′OMe modified RNA, agcu represent 2′F modified RNA, * = PS, -L represent conjugated GalNAc conjugation

The ex vivo delivery efficiency of the conjugates in the invention was tested in liver cell by RT-qPCR. The procedure of primary mouse hepatocytes isolation is as follows:

### Part A: Perfusion

(1) Perfuse with buffer A
(2) Perfuse with buffer B
(3) Dissect out liver, place into buffer C
Buffer A: Add 93 mg of EDTA (0.5 mM) to 500 mL HBSS
Buffer B: Add 400 mg of Collagenase Type-I (0.8 mg/mL)) to 500 mL DMEM
(Note: Collagenase added at the time of perfusion)
Buffer C: Add 2 mg of BSA (2%) to 100 mL DMEM

### Part-B: Isolation

After perfusion, in hood, place liver into 10 cm TC dish, open liver sack, help dissociation by shaking tissue with forceps.
Filter through 70 um filter; wash filter with buffer C, centrifuge 50 g for 5 minutes at 4° C.
Discard supernatant, resuspend gently in 50 ml buffer C (wash 1), and centrifuge at 50 g for 5 minutes at 4° C.
Discard supernatant, resuspend gently in 50 ml buffer C (wash 2), and centrifuge at 50 g for 5 minutes at 4° C.
Discard supernatant, resuspend gently in 50 ml buffer C (wash 3), and centrifuge at 50 g for 5 minutes 4° C.
Discard supernatant, resuspend in thawing/plating medium.
Count with trypanblue to assess viability/yield.
Seed the cells by using mouse primary hepatocytes thawing media (thermos fisher, CM3000) on collagenase coated plates, thermos fisher, A1142802. Better seed 1 mL/well in 24 will plate.
After 3-4 hours, replace the media with primary hepatocyte maintenance media, thermofisher, CM4000.

ex vivo Self Delivery assay was conducted without using transfection reagent (tested in 12 well plate, 100,000 cells/well, 48 hours incubation). Tested Oligo GalNAc conjugate concentrations was marked in each examples as below. The “mock” sample of Oligo GalNAc conjugate concentration is 0 nm. The expression level of targeted mRNA was detected by RT-q PCR.

For in vivo test, all animals were acclimated in-house at least for 48 h prior to study start. Female C57BL/6 mice 6-8 weeks of age were obtained from Charles River Laboratories and randomly assigned to each group. All animals were treated in accordance with IACUC protocols. Mice were dosed subcutaneously at 20 mg/Kg or 2 mg/Kg in different examples with aiRNA duplex, or phosphate buffered saline (PBS) control. Livers were harvested for efficacy analysis.

### Example 6 Ex Vivo Uptake of the aiRNA by Hepatocytes with/without Conjugates

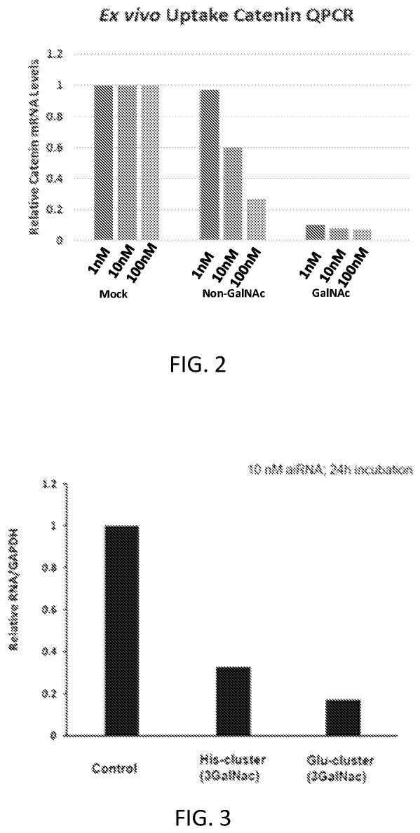

“His-cluster” conjugated mβ-Catenin aiRNA (aiRNA #1) showed remarkable gene silencing activity in self delivery ex vivo test at 100 nM, 10 nM, and even 1 nM, compared with non-conjugated aiRNA, demonstrating that the GalNAc conjugates provided by this invention have great potency for delivering duplex RNAi agent, such as aiRNA. The results were detected by QPCR as shown in FIG. 2.

### Example 7 Ex Vivo Uptake of the aiRNA by Hepatocytes with GalNac Conjugates

mβ-Catenin aiRNA (aiRNA #1) conjugated with two different GalNAc conjugation provided by this invention were cocultured with isolated mouse hepatocytes for 24 hours at 10 nM concentration. Both Glu-cluster(3 GalNAc) and His-cluster(3 GalNAc) showed very potent gene silencing in the ex vivo self-delivery w GalNAc conjugates provided by this invention have great potency for delivering duplex RNAi agent, such as aiRNA.

### Example 8 In Vivo Delivery Potency of the aiRNA Conjugated with GalNac Conjugates

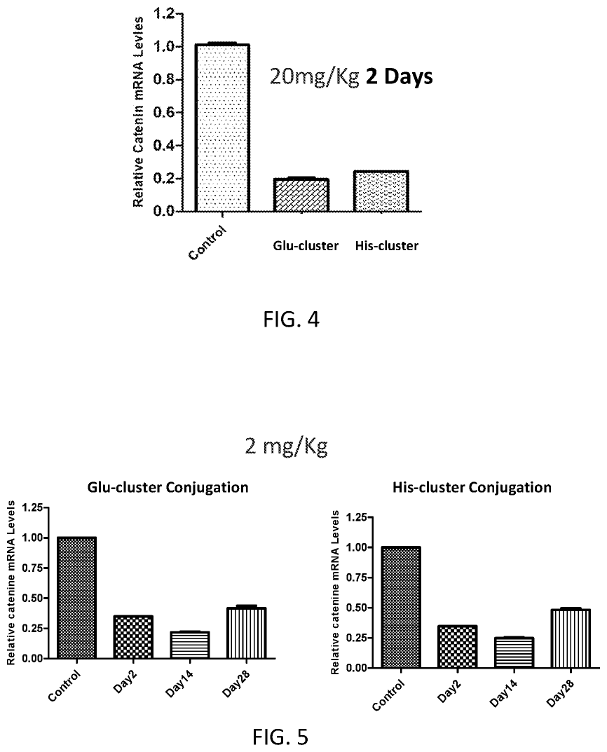

aiRNA conjugates mβ-Catenin (aiRNA #1) conjugate with “His-cluster” and “Glu-cluster” were injected subcutaneously at 20 mg/kg in C57BL/6J mice. Liver samples were collected on day 2 after injection, catenin expression levels were analyzed. As shown in FIG. 4. His-cluster as well as Glu-cluster conjugates induced potent gene silencing activity in vivo. Similar to ex vivo data collected previously, Glu-cluster conjugate appears to be slightly more potent than His-cluster in this test for delivering the said aiRNA. The gene expression in the liver was also analyzed on day 2, day 14 and day 28 after injection at dose of 2 mg/kg of the conjugated aiRNA targeting beta-catenin. As shown in FIG. 5, aiRNA conjugated through glu-cluster or as his-cluster induced potent as well as durable gene silencing activity in vivo, demonstrating that the GalNAc conjugates provided by this invention can highly efficiently delivering duplex RNAi in vivo.

### Example 9 Ex Vivo Uptake and In Vivo Delivery Efficiency of the aiRNA Conjugated with GalNac Conjugates

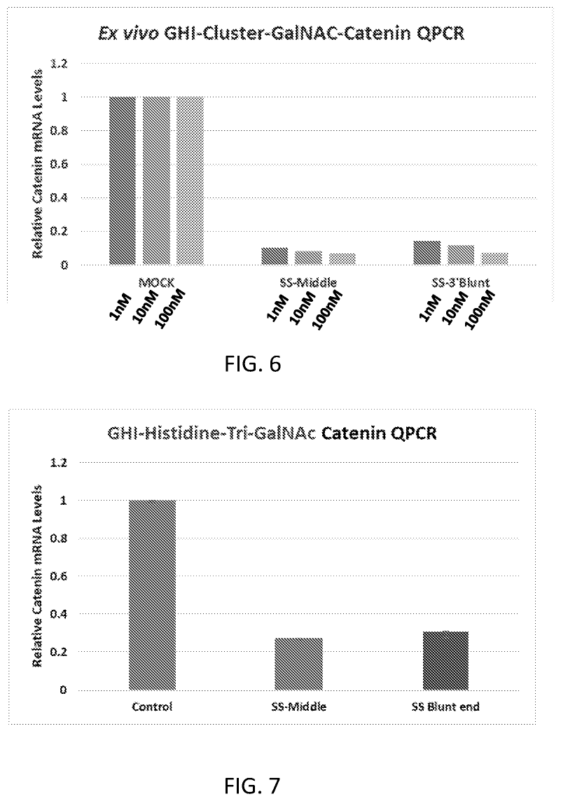

Structures of SS middle position aiRNA (aiRNA #1) and SS 3′ blunt end aiRNA (aiRNA #2) were tested ex vivo and in vivo as method described above. SS middle position aiRNA is an exemplary middle type interfering RNA duplex molecule, and SS 3′ blunt end aiRNA is an exemplary blunt end type interfering RNA duplex molecule. Both types of aiRNAs conjugated through compositions of this invention showed potent gene silencing activity in the ex vivo self-delivery assay at 100 nM, 10 nM, and even 1 nM, and in vivo (compared with control 4 h hours after single dose 23 mg/kg s.c.). The results were tested by QPCR and shown in FIG. 6 and FIG. 7. It is also surprising that the middle type showed even more potent activity than the blunt-ended configuration in both ex vivo and in vivo. These data suggest the novel linker compositions provided by this invention can be used for conjugating GalNac to different duplex RNA structures. Moreover, the middle type interfering RNA duplex/aiRNA-GalNac complex conjugated through the compositions provided in this invention may further improve gene silencing efficiency.

These results in Examples 5-9 have clearly demonstrated that GalNAc conjugate designs based on present invention can dramatically enhance the delivery efficiency of an oligonucleotide both ex vivo and in vivo, achieving great gene silencing potency for targeting different genes in hepatocyte. Moreover, the linker compositions provided in this invention were based on our body's amino acids, which eliminate the safety risk of other types of linkers used in GalNAc conjugates.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Throughout this application, various publications, patents, and/or patent applications are referenced in order to more fully describe the state of the art to which this invention pertains. The disclosures of these publications, patents, and/or patent applications are herein incorporated by reference in their entireties to the same extent as if each independent publication, patent, and/or patent application was specifically and individually indicated to be incorporated by reference.

What is claimed is:

1-68. (canceled)

69. A compound having the structural formula (G-G1): 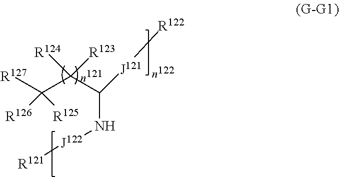
wherein:
R¹²³, R¹²⁴, R¹²⁵, R¹²⁶ are each independently for each occurrence H;
R¹²¹, R¹²² are each independently for each occurrence selected from the group consisting of OH, a protecting group for OH, a phosphate group, a phosphodiester group, an activated phosphate group, an activated phosphite group, a phosphoramidite, a solid support, OP(Z′)(Z″)O-nucleoside, OP(Z′)(Z″)O-oligonucleotide, a lipid, a PEG, a steroid, a polymer, O-nucleotide, a nucleoside, OP(Z′)(Z″)O—R^{128B}—OP(Z′″)(Z″″)O-oligonucleotide, and an oligonucleotide;
Z′, Z″, Z′″ and Z″″ are each independently for each occurrence O or S;
J¹²¹, J¹²², are each independently for each occurrence a spacer, the spacer being an alkylene of 1 to 10 carbon atoms where one or more carbon atoms are optionally replaced with one or more substituents selected from the group consisting of: C(O), NH, O, S, OP(O)O, OP(S)O, CH═N and S(O)₂, and wherein the spacer is optionally substituted by at least one of C₁-C₅ alkyl, or O—C₁-C₅ alkyl;
R¹²⁷ is J^{123A}-R^{128A} wherein J^{123A} is selected from the group consisting of: C(O), OP(O)O, OP(S)O, CH═N, and S(O)₂; and R^{128A} is R^{128C}-branching group-(R^{128B}-R^{128L})ₙ^{121L}, or R^{128B}-R^{128L}, wherein R^{128C} is selected from Table 9 as follows:

| | |
|---|---|
| 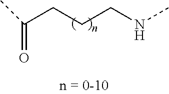 | 9-1 |
| 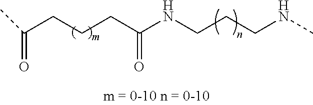 | 9-2 |
| 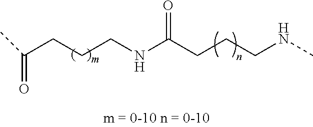 | 9-3 |
| 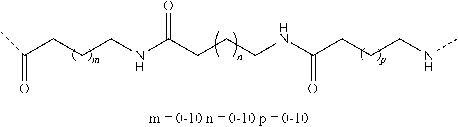 | 9-4 |
| 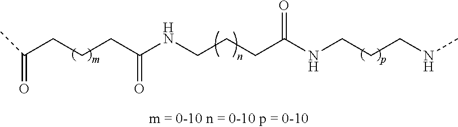 | 9-5 |
| 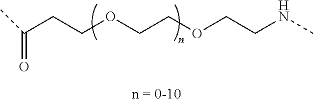 | 9-6 |
| 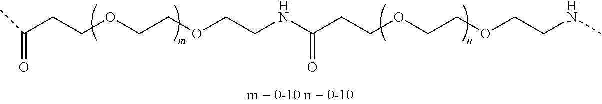 | 9-7 |
| 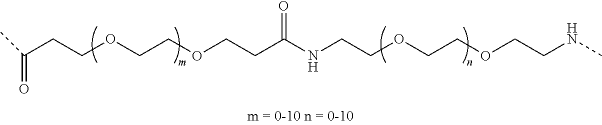 | 9-8 |
| 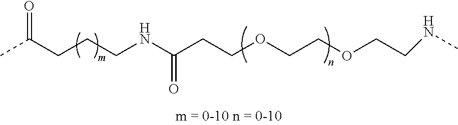 | 9-9 |
| 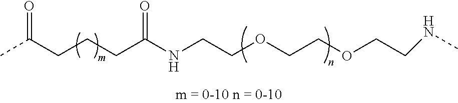 | 9-10 |
| 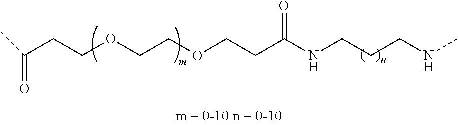 | 9-11 |
| 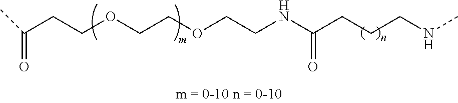 | 9-12 |
| 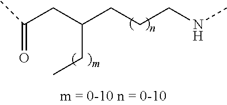 | 9-13 |

R^{128B} is independently selected from an alkylene of 1 to 30 carbon atoms, wherein one or more carbon atoms are optionally replaced with one or more substituent selected from the group consisting of: C(O), NH, O, S, OP(O)O, OP(S)O, CH═N, S(O)₂, C₂-C₁₀ alkenylene, C₂-C₁₀ alkynylene, C₆-C₁₀ arylene, C₃-C₁₈ heterocyclylene, and C₅-C₁₀ heteroarylene;
R^{128L} is independently selected from a ligand capable of docking to a cell surface receptor;
the branching group is selected from group consisting of: 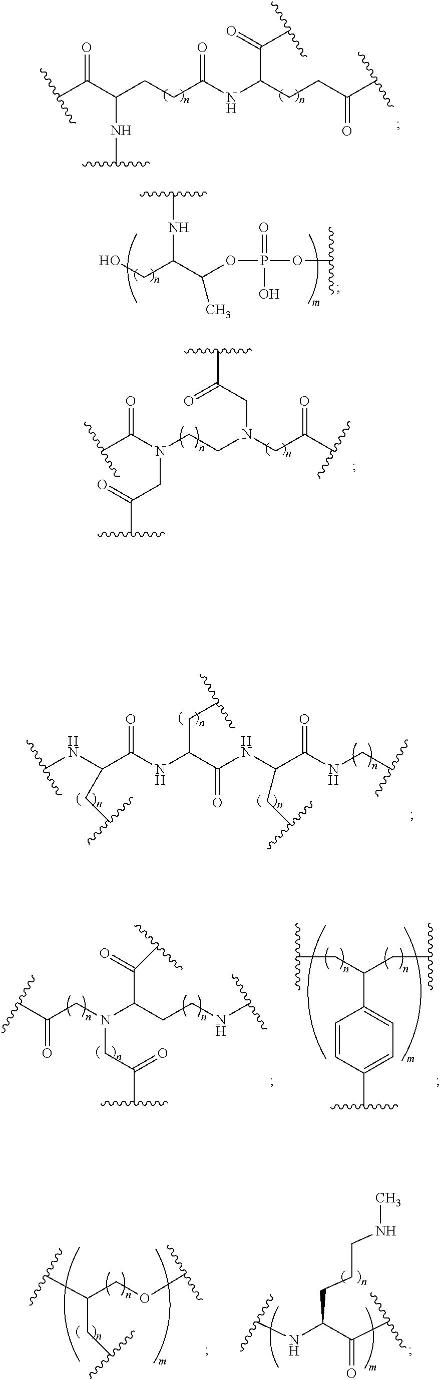 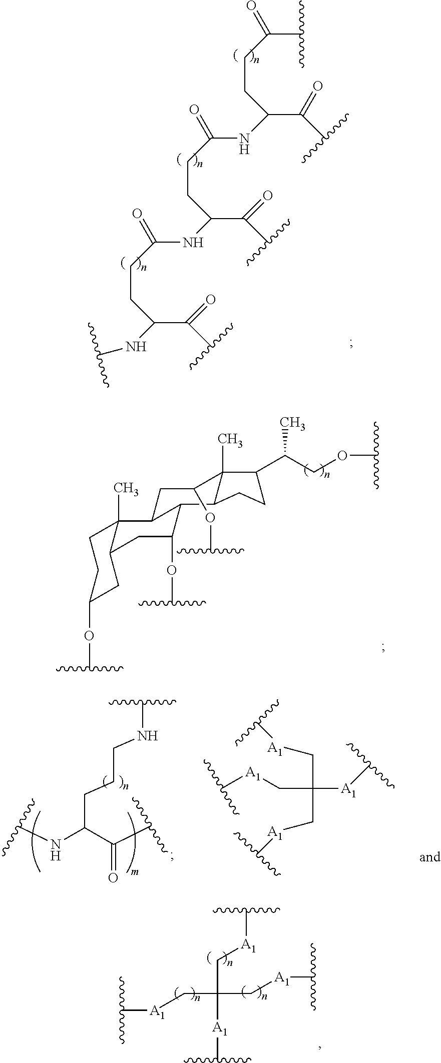
wherein each A₁ is independently, O, S, C═O, or NH; each n is independently an integer from 1 to 20; and m is an integer from 2 to 6;
n^{121L} is selected from the group consisting of 1, 2, 3, 4 and 5;
n¹²¹ is selected from the group consisting of 1, 2, 3, 4 and 5;
n¹²² is 1; and
the oligonucleotide comprises naturally occurring and/or chemically modified nucleotides/nucleosides.

70. The compound of claim 69, wherein the J^{123A} is C(O), R^{128C} is selected from the group consisting of: NH—CH₂—(CH₂)ₙ—CH₂—C(O), NH—CH₂—(CH₂)ₙ—CH₂—NHC(O)—CH₂—(CH₂)ₘ—CH₂—C(O), and NH—CH₂—(CH₂)ₙ—CH₂—C(O)NH—CH₂—(CH₂)ₘ—CH₂—C(O), and wherein the n is an integer from 0 to 10, and m is an integer from 0 to 10.

71. The compound of claim 69, wherein the branching group is selected from group consisting of:

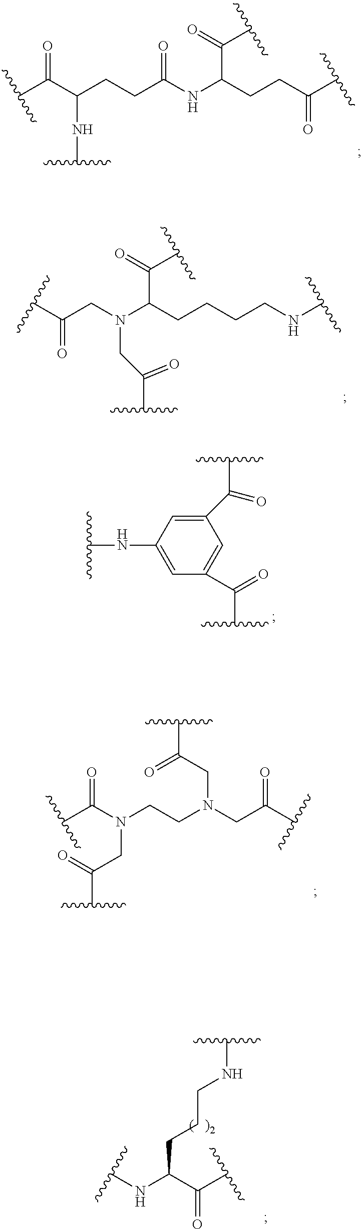

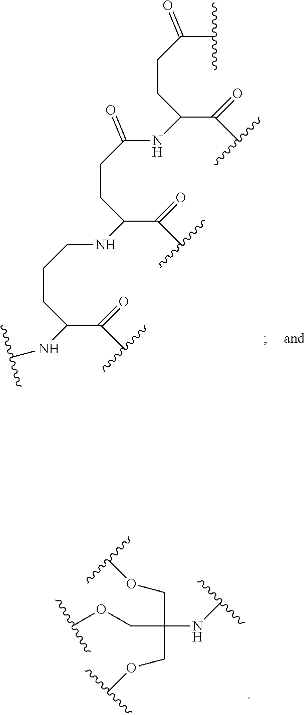 [SMILES: *NC(CCCNC(CCC(=O)NC(CCC(*)=O)C(*)=O)C(*)=O)C(*)=O ; *NC(CO*)(CO*)CO*]

72. The compound of claim 69, wherein each of the ligand in R^{128L} is independently selected from the group consisting of N-acetyl galactosamine (GalNAc), N-Ac-Glucosamine (GluNAc), galactose, lactose, mannose, cholesterol, tocopherol, biotin, cyanine dyes, folic acid, RGDp, transferrin, anisamide, lactobionic acid, cRGD, hyaluronic acid, low molecular weight protamine, lipid derivatives, peptides, cyclic peptides, and heterocycles.

73. The compound of claim 69, wherein the R^{128B} is selected from Table 4 as follows:

| | |
|---|---|
| 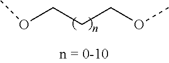 | 6-1 |
| 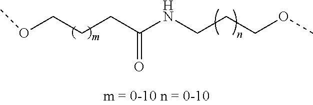 | 6-2 |
| 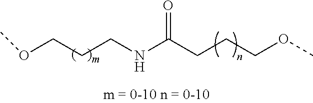 | 6-3 |
| 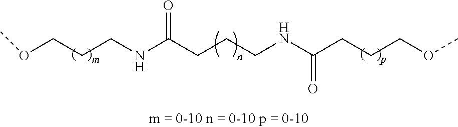 | 6-4 |
| 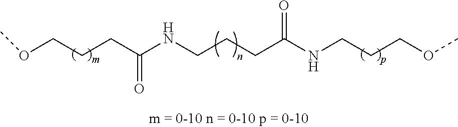 | 6-5 |
| 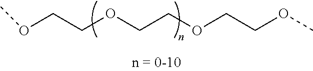 | 6-6 |
| 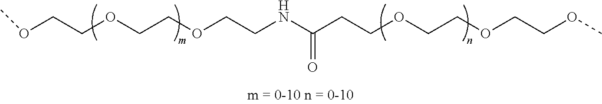 | 6-7 |
| 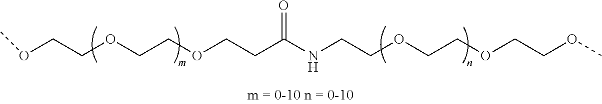 | 6-8 |
| 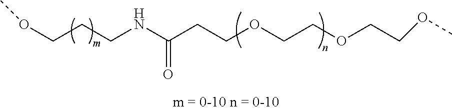 | 6-9 |
| 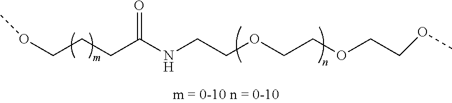 | 6-10 |
| 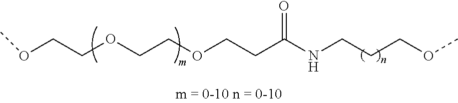 | 6-11 |
| 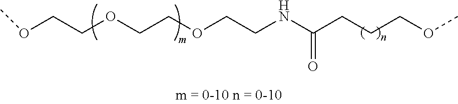 | 6-12 |
| 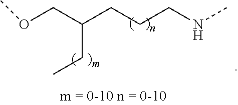 | 6-13 |

74. The compound of claim 69, wherein the n¹²¹ is 1.

75. The compound of claim 69, wherein the n^{121L} is 3.

76. The compound of claim 69, having the structural formula (G-G1-09) or (G-G1-10) as follows: 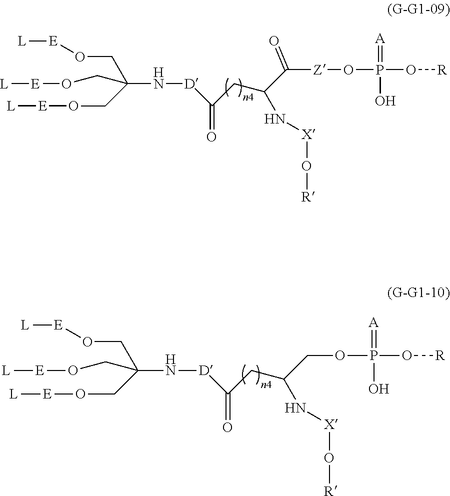
wherein:
R comprises an oligonucleotide formed by naturally occurring and/or chemically modified nucleotides/nucleosides;
R′ is selected from the group consisting of an oligonucleotide formed by naturally occurring and/or chemically modified nucleotides/nucleosides, H and a protecting group;
A is O or S;
D′ is selected from Table 9;
each E is R^{128B} independently selected from an alkylene of 1 to 30 carbon atoms, wherein one or more carbon atoms are optionally replaced with any one or more substituent selected from the group consisting of: C(O), NH, O, S, OP(O)O, OP(S)O, CH═N, S(O)₂, C₂-C₁₀ alkenylene, C₂-C₁₀ alkynylene, C₆-C₁₀ arylene, C₃-C₁₈ heterocyclylene, and C₅-C₁₀ heteroarylene;
X′ is J¹²², a spacer being an alkylene of 1 to 10 carbon atoms, wherein one or more carbon atoms are optionally replaced with any one or more substituent selected from the group consisting of: C(O), NH, O, S, OP(O)O, OP(S)O, CH═N and S(O)₂, and wherein the spacer is optionally substituted by at least one of C₁-C₅ alkyl, or —OC₁-C₅ alkyl;
Z′ is independently selected from Table 2 as follows:

| | |
|---|---|
| 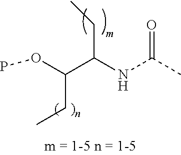 | 4-1 |
| 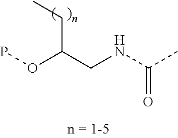 | 4-2 |
| 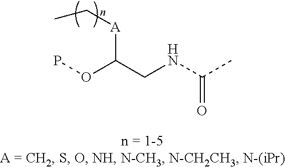 | 4-3 |
| 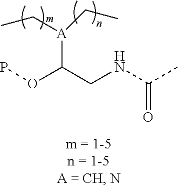 | 4-4 |
| 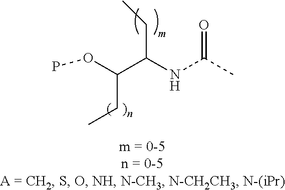 | 4-5 |
| 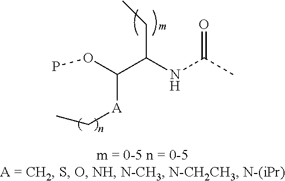 | 4-6 |
| 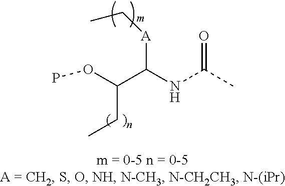 | 4-7 |
| 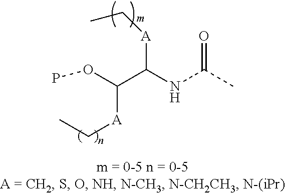 | 4-8 |
| 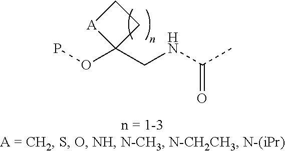 | 4-9 |
| 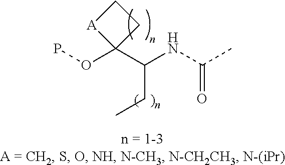 | 4-10 |
| 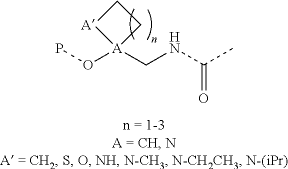 | 4-11 |
| 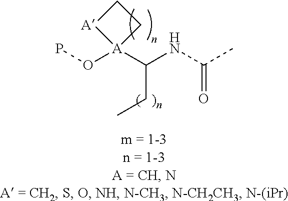 | 4-12 |
| 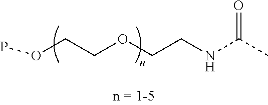 | 4-13 |
| 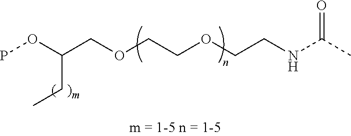 | 4-14 |
| 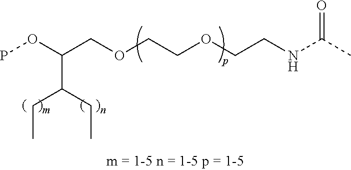 | 4-15 |
| 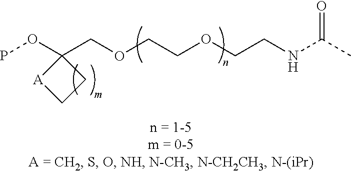 | 4-16 |
| 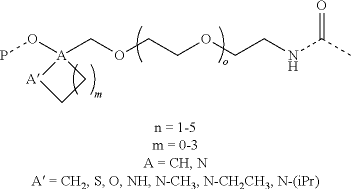 | 4-17 |
| 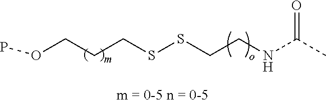 | 4-18 |
| 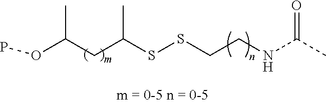 | 4-19 |
| 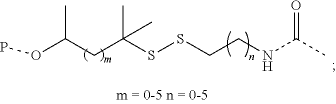 | 4-20 |

each L independently comprises a ligand moiety capable of docking to a cell-surface receptor; and
n4 is independently selected from the group consisting of 1, 2, 3 and 4.

77. The compound of claim 76, wherein each E is selected from Table 4 as follows:

| | |
|---|---|
| 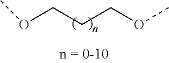 | 6-1 |
| 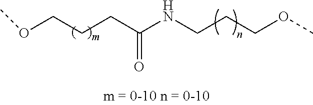 | 6-2 |
| 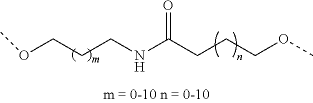 | 6-3 |
| 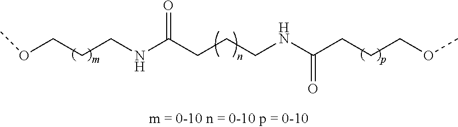 | 6-4 |
| 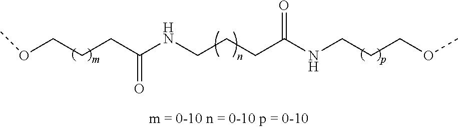 | 6-5 |
| 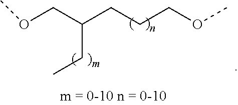 | 6-6 |
| 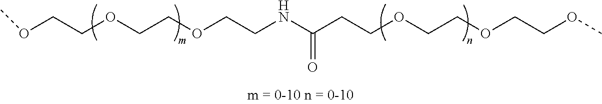 | 6-7 |
| 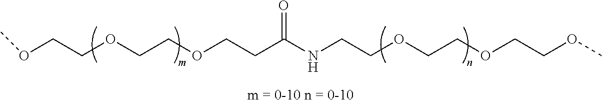 | 6-8 |
| 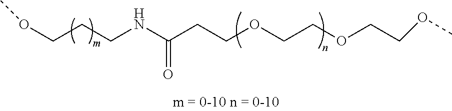 | 6-9 |
| 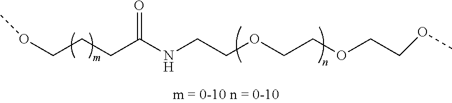 | 6-10 |
| 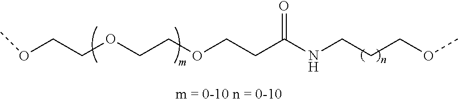 | 6-11 |
| 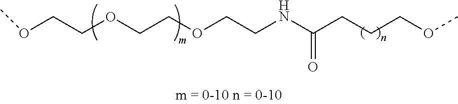 | 6-12 |
| 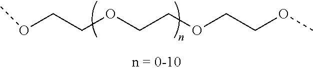 | 6-13 |

78. The compound of claim 76, wherein the n4 is 2, D is selected from the group consisting of: NH—CH₂—(CH₂)ₙ—CH₂—C(O), NH—CH₂—(CH₂)ₙ—CH₂—NHC(O)—CH₂—(CH₂)ₘ—CH₂—C(O), and NH—CH₂—(CH₂)ₙ—CH₂—C(O)NH—CH₂—(CH₂)ₘ—CH₂—C(O), the n being an integer from 0 to 10 and the m being independently an integer from 0 to 10.

79. The compound of claim 69, having the structural formula 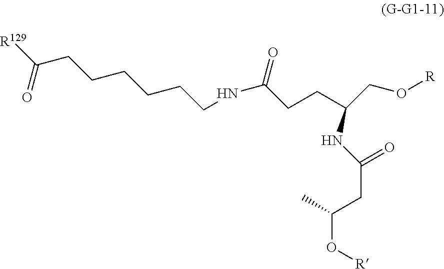
wherein:
R¹²⁹ has the structure of -branching group-(R^{128B}-R^{128L})ₙ^{121L};
R comprises an oligonucleotide formed by naturally occurring and/or chemically modified nucleotides/nucleosides; and
R′ is selected from the group consisting of a solid support, an oligonucleotide comprising natural or chemically modified nucleotides/nucleosides, H and a protecting group.

80. The compound of claim 79, wherein the R^{128B} is selected from Table 4 as follows:

| | |
|---|---|
| 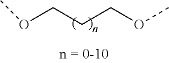 | 6-1 |
| 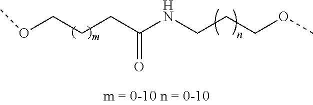 | 6-2 |
| 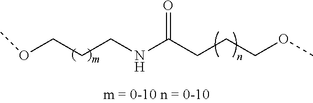 | 6-3 |
| 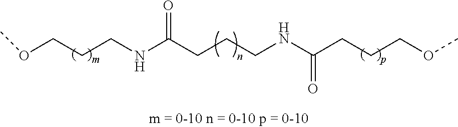 | 6-4 |
| 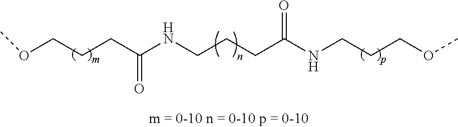 | 6-5 |
| 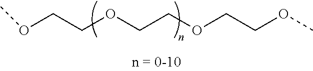 | 6-6 |
| 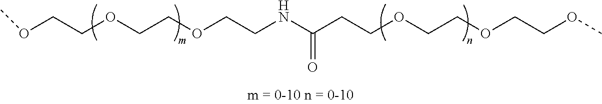 | 6-7 |
| 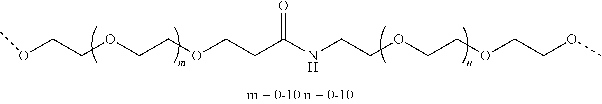 | 6-8 |
| 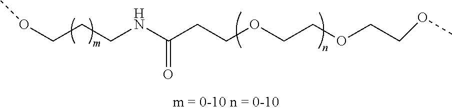 | 6-9 |
| 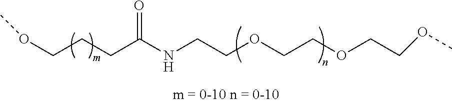 | 6-10 |
| 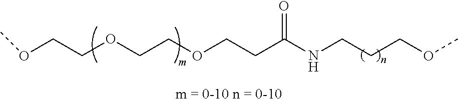 | 6-11 |
| 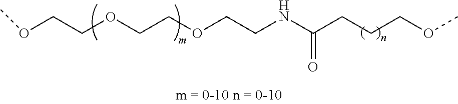 | 6-12 |
| 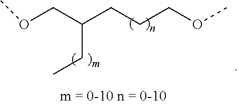 | 6-13 |

81. The compound of claim 79, wherein the R^{128L} is independently a ligand capable of docking to a cell surface receptor, wherein the ligand is selected from the group consisting of N-acetyl galactosamine (GalNAc), N-Ac-Glucosamine (GluNAc), galactose, lactose, mannose, cholesterol, tocopherol, biotin, cyanine dyes, folic acid, RGDp, transferrin, anisamide, lactobionic acid, cRGD, hyaluronic acid, low molecular weight protamine, lipid derivatives, peptides, cyclic peptides, and heterocycles.

82. The compound of claim 79, wherein the ₙ^{121L} is 3, and the branching group is selected from the group consisting of

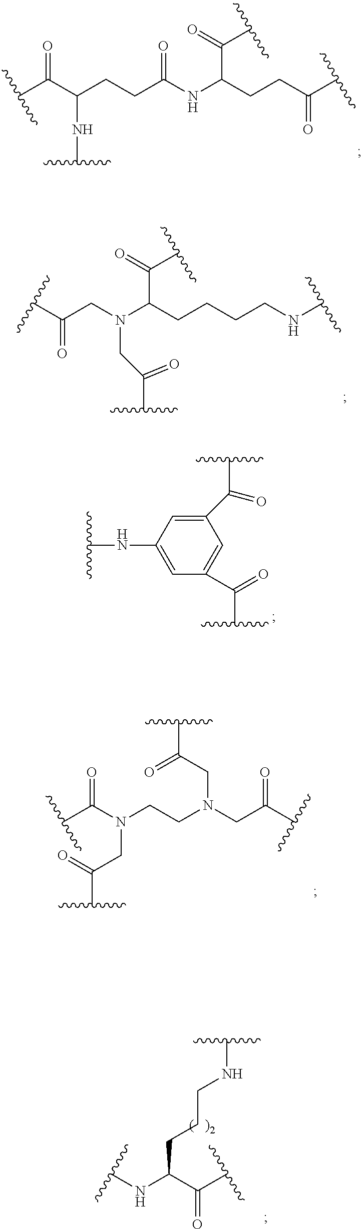

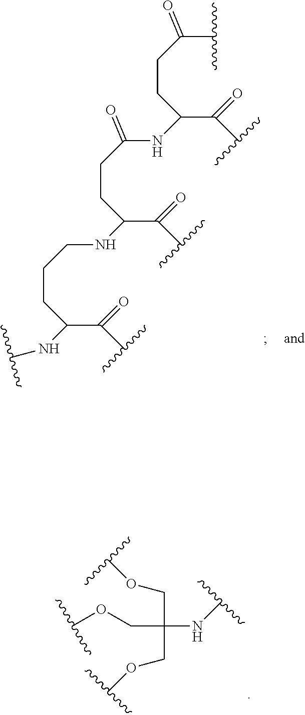 [SMILES: *NC(CCCNC(CCC(=O)NC(CCC(*)=O)C(*)=O)C(*)=O)C(*)=O ; *NC(CO*)(CO*)CO*]

83. The compound of claim 69, wherein the compound has the structure selected from the group shown in formula GC-1 to GC-9 as follows:

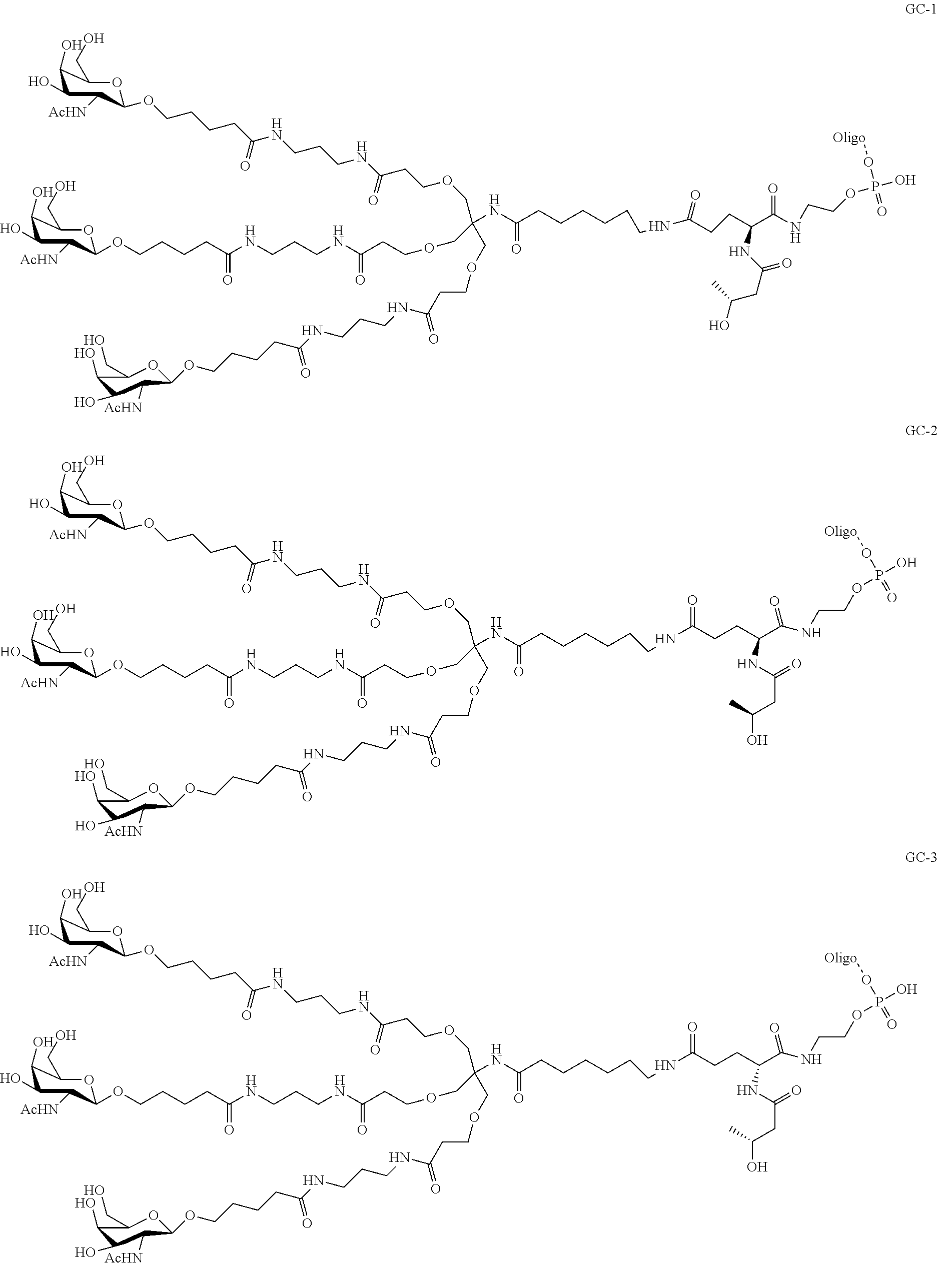

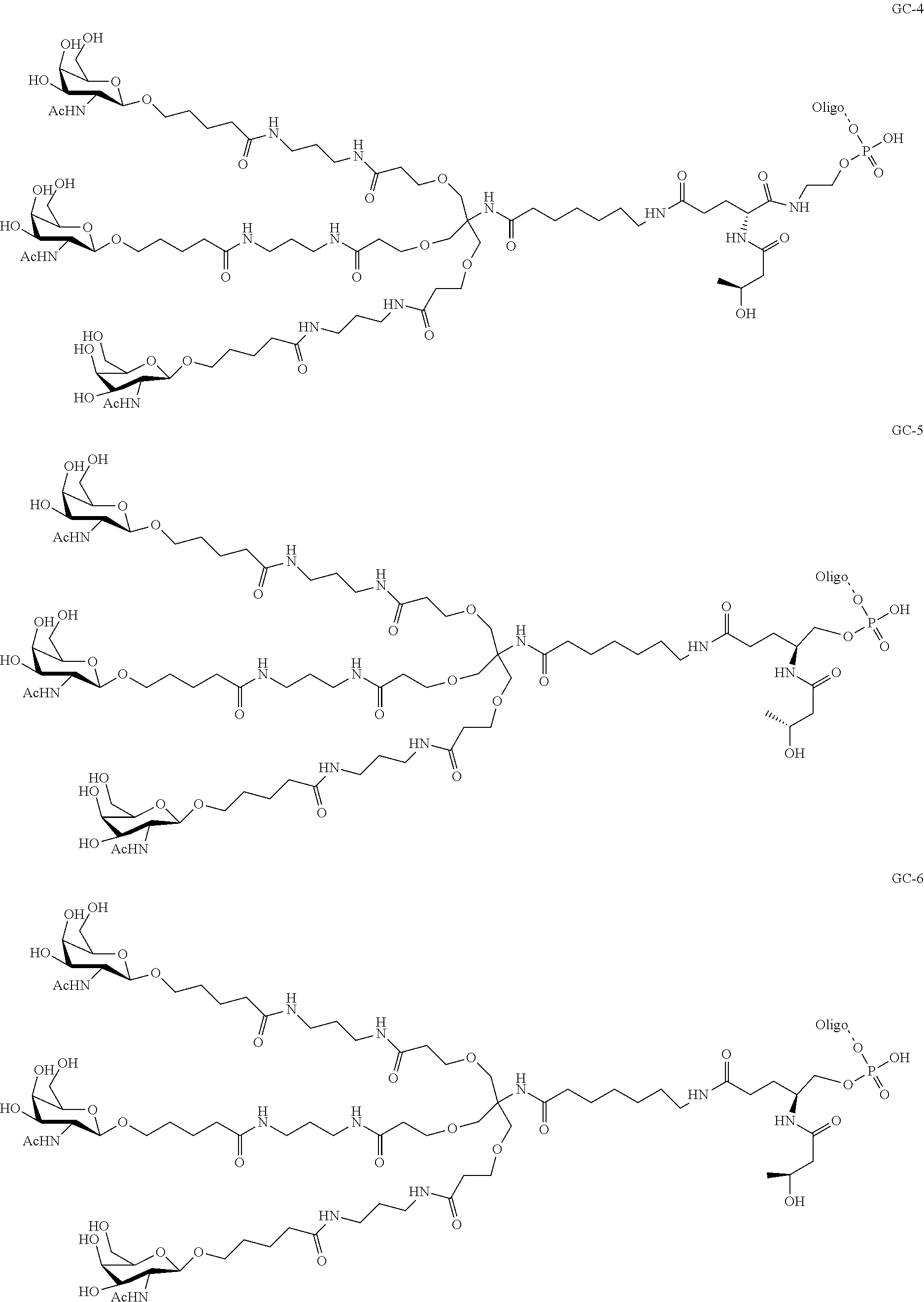

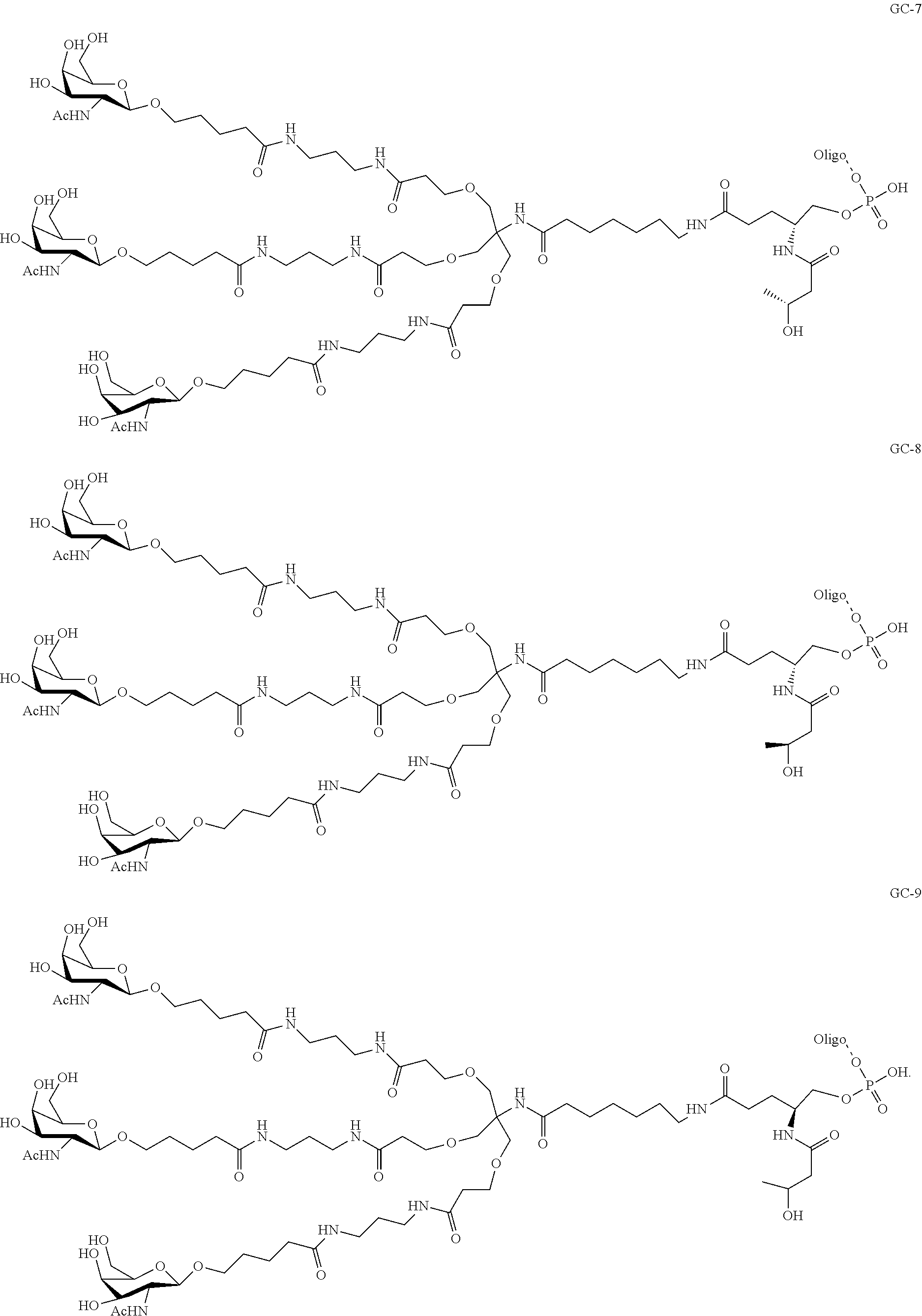

84. The compound of claim 69, wherein the oligonucleotide is linked to the rest of the compound through its 5′ end and/or 3′ end.

85. The compound of claim 84 wherein the oligonucleotide comprises a molecule selected from the group consisting of a small interfering RNA (siRNA) duplex, an asymmetric interfering RNA (aiRNA) duplex, an antisense oligonucleotide (ASO), and a micro-RNA (miRNA).

86. The compound of claim 85, wherein the aiRNA comprising an antisense strand and a sense strand, wherein:
(a) the antisense strand is longer than the sense strand, has a length of 19, 20, 21, 22, 23, 24, 25, 26 or 27 nucleotides and includes a 3′-overhang of 1-9 nucleotides and a 5′-overhang of 0-8 nucleotides when duplexed with the sense strand; and
the sense strand has a length of 12, 13, 14, 15, 16, 17, 18, 19, 20, 21, 22, 23, 24, 25 or 26 nucleotides and forms a double-stranded region with the antisense strand;
(b) the antisense strand is longer than the sense strand, has a length of 19, 20, 21, 22, 23, 24, 25, 26 or 27 nucleotides and includes a 3′-overhang of 1-9 nucleotides and a 5′-overhang of 1-8 nucleotides when duplexed with the sense strand; and
the sense strand has a length of 12, 13, 14, 15, 16, 17, 18, 19, 20, 21, 22, 23, 24, 25 or 26 nucleotides and forms a double-stranded region with the antisense strand;
or
(c) the antisense strand is longer than the sense strand, has a length of 19, 20, 21, 22, 23, 24, 25, 26 or 27 nucleotides and includes a 3′-overhang of 1-9 nucleotides and a 5′ blunt end when duplexed with the sense strand; and
the sense strand has a length of 12, 13, 14, 15, 16, 17, 18, 19, 20, 21, 22, 23, 24, 25 or 26 nucleotides and forms a double-stranded region with the antisense strand.

87. A pharmaceutical composition comprising a compound of claim 69 and a pharmaceutically acceptable excipient, carrier, or diluent.

88. A method for treating a disease or condition in a subject comprising administrating to subject an effective amount of the pharmaceutical composition of claim 87.


SEQUENCE LISTING (3 sequences)

SEQ ID NO: 1 - RNA, 17 nt, Artificial Sequence
  Artificially synthesized oligonucleotide sequence
aguggauucuguacugu

SEQ ID NO: 2 - RNA, 21 nt, Artificial Sequence
  Artificially synthesized oligonucleotide sequence
aaacaguacagaauccacugg

SEQ ID NO: 3 - RNA, 17 nt, Artificial Sequence
  Artificially synthesized oligonucleotide sequence
uggauucuguacuguuu